(19) United States
(12) Patent Application Publication
(10) Pub. No.: US 2020/0049722 A1
(43) Pub. Date: Feb. 13, 2020

(54) DIAGNOSIS, PROGNOSIS AND TREATMENT FOR SCHIZOPHRENIA AND SCHIZOAFFECTIVE PSYCHOSIS

(71) Applicant: Precision Medicine Holdings Pty Ltd, Unley (AU)

(72) Inventors: Stephanie Fryar-Williams, Unley (AU); Graeme Tucker, Hope Valley (AU)

(21) Appl. No.: 16/335,996

(22) PCT Filed: Sep. 26, 2017
(86) PCT No.: PCT/AU2017/051049

§ 371 Date: Mar. 22, 2019

(30) Foreign Application Priority Data
Sep. 26, 2016 (AU) 2016903895

(51) Int. Cl. G01N 33/82; C12Q 1/6883

(52) U.S. Cl. CPC G01N 33/82; C12Q 1/6883; G01N 2800/52; C12Q 2600/156; G01N 2800/302; C12Q 2600/112

(57) ABSTRACT

Provided herein are methods for diagnosing a psychotic disorder, such as schizophrenia, schizoaffective disorder or psychosis, predicated on a determination of a methylation phenotype of the subject based in part on the identity of a specific polymorphism in the MTHFR gene and the analysis of a suite of biomarkers and other functional measures and indices. Also provided herein are methods for predicting prognosis and functional outcomes for subjects having a psychotic disorder, and for treating subjects having a psychotic disorder.

### FIELD OF THE INVENTION

The present invention relates to a novel set of genetic, biochemical and sensory processing markers for the diagnosis of, and prediction of susceptibility to, schizophrenia, schizoaffective disorder and psychosis, and to methods for the diagnosis of, and prediction of susceptibility to, schizophrenia, schizoaffective disorder and psychosis employing these biomarkers. Also contemplated is the use of these markers in identifying subtypes of psychosis that provide substrates for subtype-specific treatment regimens.

### BACKGROUND OF THE INVENTION

Psychotic disorders are a group of serious mental illnesses with an age of onset in late adolescence, early adulthood and adulthood. The most common psychotic disorders include schizophrenia, bipolar disorder with psychotic features, and major depression with psychotic features. Psychotic disorders are primarily characterized by the presence of hallucinations, delusions (such as paranoia) and perceptual changes such as hearing voices and related changes in mood, demeanor and behaviour.

Schizophrenia represents the majority of psychotic disorders (about 60%). In 1990, schizophrenia was estimated to be the 10th leading cause of non-fatal burden in the world, accounting for 2.6% of total “years of life lived with disability” (YLD), around the same percentage as congenital malformations. The Global Burden of Disease 2000 study found the disease to be the 7th leading cause of YLDs at the global level, accounting for 2.8% of total global YLD.

According to Diagnostic and Statistical Manual of Mental Disorder-IV (DSM-IV) (and the current DSM V), the essential features of schizophrenia consist of a mixture of characteristic signs and symptoms that have been present for a significant length of time during a 1-month period with some signs of the disorder persisting for at least 6 months. However no single symptom is characteristic of the disease. Moreover, recognition of the heterogeneity of schizophrenia and psychosis has led to increasing dissatisfaction with currently used classification systems.

Patients with schizophrenia onset have been found to have the longest duration of untreated psychosis. They also experience recurrent or treatment resistant symptoms with the finding that three-quarters of patients presenting with first non-affective psychotic symptoms achieved recovery within 2 years, but only 41% returned to baseline functioning, and nearly half experienced new episodes. In order to lower the risk of treatment delay due to diagnostic uncertainty or symptoms continuing or being added to by increased perturbations in linked biological systems new intervention and biological disease progression prevention therapies are needed with minimal risk of unwanted side-effects and sustained therapeutic effect. Therefore in the last few years, there have been calls for new biological approaches such as consideration of the role of gut microbiome and dietary needs for management of mental illness. Supplementation with vitamin D, choline, serine and omega fatty acids have been recommended, however such recommendations occur in a setting where no clear integrated biological framework linking biomarkers with symptoms and diagnostic certainty and has fully emerged and subtypes of psychosis are not yet fully-clarified. Thus it is not yet known whether gut, nutritional or dietary treatments (like antipsychotic medications) also represent a “one size fits all” approach and whether they may do “more harm than good”, in individuals with differing underlying biology.

Current diagnostic approaches are typically descriptive or rely predominantly on symptomatic analyses based on, for example, physical examination, gross medical evaluation, psychological and/or psychiatric evaluation, anecdotal family history, and emotional history. Heterogeneity of the disease etiology and presentation, however, contributes to the difficulty in diagnosis of schizophrenia and psychosis.

Neuroimaging studies have detected brain changes over time from first episode psychosis (FEP) to chronic schizophrenia, yet no firm progression pathway has as-yet been established for predicting prognosis, duration of illness and/or stages of its development based upon of symptom-behaviour complexes and biological measures. Diagnoses that vary considerably over time and evidence that schizophrenia is not a unitary condition, but part of a spectrum with bipolar disorder have led to calls for models that better predict expected duration of illness (DOI) using gene-related neurobiological means related to functional outcomes.

Hostility and suicidality are profound thought and behaviour problems within the architecture of psychosis. These symptoms and behaviours are often clinically difficult to predict and difficult to manage. They pose risks to patient and carers alike and there is currently no suite of biological markers able to characterise them.

There is a clear need for improved, objective, neuroscience-based methods for diagnosing schizophrenia and psychosis and identifying biological substrates of the disorder. This would be greatly facilitated by the identification of markers for schizophrenia and psychosis, along with indicators of potentially-preventable susceptibility or risk factors and functional prognostic outcome measures, enabling the development of accurate, sensitive and easy to employ diagnostic tests and personalized treatments.

Currently available strategies for treating and preventing schizophrenia and psychosis typically involve attenuating symptoms with pharmacological psychiatric interventions (e.g., antipsychotics, antidepressants, mood stabilizers), which are not ideal. More effectively targeted, personalised, precision treatment strategies based on an individual patient's genotype and individual biochemical phenotype would lead to the development of more effective treatment strategies, would assist remission and recovery and would assist clinicians with relapse prevention and management of treatment-resistance.

### SUMMARY OF THE INVENTION

The inventors have explored biomarkers for psychosis in schizophrenia and schizoaffective disorder against a background of relationships between molecules and elements within biochemical pathways. The inventors have identified novel methods of diagnosis, described herein, utilizing different methylation signatures within a patient's MTHFR 677 genotype to identify different psychosis subtypes that may be used to determine treatments that specifically target the patient's underlying biology.

In a first aspect of the present invention there is provided a method for diagnosing a psychosis phenotype in a subject with a psychotic disorder, the method comprising:
(a) obtaining one or more biological samples from said subject, and
(b) determining the status of the C677T polymorphism of the MTHFR gene from the one or more biological samples,
wherein
   (i) the presence of the homozygous CC genotype at position 677 of the MTHFR gene is indicative of an under-methylating psychosis phenotype,
   (ii) the presence of the homozygous TT genotype at position 677 of the MTHFR gene is indicative of a low activity MTHFR enzyme and an over-methylating psychosis phenotype, and
   (iii) the presence of the heterozygous CT genotype at position 677 of the MTHFR gene is indicative of a mixed-methylation psychosis phenotype.

The subject may or may not be known to have the psychotic disorder prior to said diagnosis. Typically the psychotic disorder is schizophrenia, schizoaffective disorder or psychosis.

The method typically also comprises the determination of levels of one or more biomarkers as described herein, and optionally the ratios of selected biomarkers as described herein, in the one or more biological samples, to inform the diagnosis. Such biomarkers may, in particular embodiments, be selected from one or more of: free copper, zinc, indolamines and catecholamines and their metabolites, vitamin and mineral or trace element cofactors (such as vitamin D, vitamin B2 (riboflavin), vitamin B6, vitamin B12, folate), intermediate substances, and vitamin B2 excretion levels. In exemplary embodiments, the one or more biomarkers may be selected from: free copper, zinc, vitamin D, riboflavin (vitamin B2) and flavin-related compounds such as flavin adenine dinucleotide (FAD) and flavin mononucleotide (FMN), vitamin B6, vitamin B12, folate and related compounds, S-adenosylmethionine (SAMe), S-adenosylhomocysteine (SAH), hydroxylpyrolline-2-one (HPL), histamine, adrenaline (AD), noradrenaline (NA), dopamine (DA), 5-hydroxyondolacetic acid (5HIAA) and methylhydroxy vanillyl-mandelic acid (MHMA).

In exemplary embodiments the biological sample(s) may comprise blood (such as whole blood, blood plasma or serum) or urine samples.

In a particular exemplary embodiment, the method comprises the measurement of riboflavin and flavin-related compounds in a urine sample. Optionally the flavin-related compounds are riboflavin metabolites, such as FAD and FMN and their degradation products. Optionally the method comprises determining the ratio of riboflavin synthesis to riboflavin degradation or the difference between riboflavin synthesis and degrdation, in the urine sample.

The method may also comprise the assessment or measurement of one or more additional parameters. Such parameters may include, but are not limited to, measurement or assessment of one or more symptom ratings for schizophrenia, schizoaffective disorder or psychosis, risk factor analysis, functional visual and auditory acuity, external ear canal patency, tympanic membrane status, motor capacity, extrapyramidal and thyroid status.

Symptom ratings may be measured or assessed using one or more psychiatric symptom rating scales known to those skilled in the art. Exemplary symptom rating scales include, but are not limited to: the Brief Psychiatric Rating Scale (BPRS); the Positive and Negative Syndrome Scale (PANSS), the Global Assessment of Function (GAF) Scale; the Clinical Global Impressions (CGI) score; and the Social and Occupational Functioning Scale (SOFAS). An index for hospital admission frequency (number of admissions/DOI) and disability pension requirement (DSP) may also be determined.

Exemplary risk factors for analysis include, but are not limited to, risk factors with regard history of ear infection, developmental disorder or delay, family history of mental illness, history of clinical or subclinical head injury, history of abuse, and history of learning disorder.

The determination of biomarker levels, or ratios thereof, and the assessment of measurement of additional parameters may be subjected to one or more statistical analyses. Exemplary statistical analyses include, but are not limited to: receiver operating characteristic (ROC) analysis, logistic regression analysis, Spearman's rank correlation analysis, and the Mann-Whitney U test.

The method may further comprise measuring the levels of one or more biomarkers as described herein, and optionally the ratios of selected biomarkers as described herein, and/or one or more of said additional parameters, in one or more control individuals, wherein said control individuals are known not to suffer from the psychotic disorder. Optionally, the MTHFR 677 genotype of the one or more control subjects are known or are determined.

Alternatively, the method may comprise comparing the levels, measured values or ratios of the one or more biomarkers, and/or the assessment of measurement of the one or more additional parameters for the subject to corresponding control biomarker levels, values or ratios, and/or to corresponding control additional parameter values, wherein the control levels, values and ratios are from a population of individuals known not to suffer from the psychotic disorder. Optionally, the MTHFR 677 genotypes of individuals in the control population are known.

In a second aspect of the present invention there is provided a method for diagnosing a psychotic disorder, optionally schizophrenia, schizoaffective disorder or psychosis, in a subject, the method comprising:
(a) obtaining one or more biological samples from said subject,
(b) determining the status of the C677T polymorphism of the MTHFR gene from the one or more biological samples,
wherein
   (i) the presence of the homozygous CC genotype at position 677 of the MTHFR gene is indicative of an under-methylating psychosis phenotype,
   (ii) the presence of the homozygous TT genotype at position 677 of the MTHFR gene is indicative of a low activity MTHFR enzyme and an over-methylating psychosis phenotype, and
   (iii) the presence of the heterozygous CT genotype at position 677 of the MTHFR gene is indicative of a mixed-methylation psychosis phenotype,
(c) determining levels of one or more biomarkers as defined in the first aspect, and optionally the ratios of selected biomarkers, and/or measuring or assessing one or more additional parameters as defined in the first aspect, and
(d) optionally comparing determined, measured or assessed levels, values or ratios from (c) with corresponding control levels, values or ratios from on or more individuals known not to suffer from the psychotic disorder.

In a third aspect of the present invention there is provided a method for diagnosing a psychotic disorder, optionally schizophrenia, schizoaffective disorder or psychosis, in a subject, the method comprising:
(a) obtaining one or more biological samples from said subject,
(b) determining levels of one or more biomarkers as defined in the first aspect, and optionally the ratios of selected biomarkers, and/or measuring or assessing one or more additional parameters as defined in the first aspect, and
(c) optionally comparing determined, measured or assessed levels, values or ratios from (b) with corresponding control levels, values or ratios from on or more individuals known not to suffer from the psychotic disorder,
wherein optionally the status of the C677T polymorphism of the MTHFR gene in the subject is known, or is determined.

In a fourth aspect of the present invention there is provided a method for predicting or determining expected duration of illness of a subject with a psychotic disorder, the method comprising:
(a) obtaining one or more biological samples from said subject,
(b) determining the status of the C677T polymorphism of the MTHFR gene and the psychosis phenotype according to the first aspect,
(c) determining levels of one or more biomarkers as defined in the first aspect, and optionally the ratios of selected biomarkers,
(d) measuring or assessing one or more additional parameters as defined in the first aspect, and
(e) determining expected duration of illness from an analysis of said determined, measured or assessed biomarkers and additional parameters.

The expected duration of illness is typically expressed in years and may be presented in the form of a duration of illness index derived by predicative algorithm or comparison of said determined, measured or assessed biomarkers and additional parameters.

Similarly, additional aspects of the invention provide for predicting or determining one or more additional functional outcomes or measures. Such functional outcomes or measures may be selected from: admission frequency; cost and/or care burden; requirement for disability support pension; symptom intensity rating; clinical global awareness of illness severity; global assessment of function score; social and occupational function scale value; hostility; and suicidality.

In a fifth aspect of the present invention there is provided a method for determining or predicting prognosis of a psychotic disorder in a subject, the method comprising:
(a) obtaining one or more biological samples from said subject,
(b) determining the status of the C677T polymorphism of the MTHFR gene and the psychosis phenotype according to the first aspect,
(c) determining levels of one or more biomarkers as defined in the first aspect, and optionally the ratios of selected biomarkers,
(d) measuring or assessing one or more additional parameters as defined in the first aspect, and
(e) determining or predicting prognosis of the disorder.

Typically the additional parameters include expected of predicted functional outcome measures for one or more of SIR, GAF, SOFAS, expected frequency of hospital admissions, CGI status, and DSP status.

In a sixth aspect of the present invention there is provided a method for treating or preventing a psychotic disorder in a subject, or for alleviating one or more symptoms of said disorder, the method comprising:
(a) determining the MTHFR 677 genotype and psychosis phenotype of a subject in accordance with the first aspect,
(b) optionally determining levels of one or more biomarkers as defined in the first aspect, and optionally the ratios of selected biomarkers,
(c) optionally measuring or assessing one or more additional parameters as defined in the first aspect, and
(d) determining a suitable treatment regime for the subject based on the above.

By way of example, where the MTHFR 677 genotype of the subject is the homozygous wild-type (CC) genotype and the psychosis phenotype of the subject is under-methylating, the method may comprise administering to the subject an effective amount of riboflavin, a prodrug analogue or derivative thereof, and/or an agent capable of inhibiting riboflavin degradation. The riboflavin may be administered in the form of one or more riboflavin-producing probiotic microorgansims, a food high in riboflavin, and/or a riboflavin supplement.

In a seventh aspect of the present invention there is provided a composition comprising riboflavin, a prodrug analogue or derivative thereof, and/or an agent capable of inhibiting riboflavin degradation, for use in the treatment or prevention of a psychotic disorder, or for the alleviation of one or more symptoms of said disorder, in a subject with an under-methylating psychosis phenotype as defined in the first aspect.

In an eighth aspect of the present invention there is provided a method for evaluating the efficacy of a treatment regime in a subject with a psychotic disorder, the method comprising:
(a) treating the subject with a treatment regime for the psychotic disorder for a period sufficient to evaluate the efficacy of the regime;
(b) obtaining one or more biological samples from the subject;
(c) determining the status of the MTHFR C677T polymorphism, and optionally determining measuring or assessing one or biomarkers and/or additional parameters as defined herein in the one or more biological samples;
(d) repeating steps (b) and (c) at least once over a period of time; and
(e) determining whether the levels, values or ratios for the one or more biomarkers and/or additional parameters change over the period of time.

### BRIEF DESCRIPTION OF THE DRAWINGS

Embodiments of the present disclosure are described herein, by way of non-limiting example only, with reference to the following figures:

FIG. 1: Interactive biochemical pathway relationships. In a bicyclic process referred to as one-carbon metabolism, the folate cycle is coupled to the methionine (methylation) cycle through the generation of 5-methyl-THF (MTHF) by the flavin dependent enzyme methyl tetrahydrofolate reductase (MTHFR). Protein for this enzyme is coded by the MTHFR 677 C->T gene where Cytosine may be replaced by Thymidine at the 677 position. Through metabolism of homocysteine (HCY) at the methionine synthase (MS) junction point between the folate and methionine cycles, MTHF donates a carbon to homocysteine to generate methionine, which in turn generates S-adenosyl methionine (SAMe). As a major methyl donor in cells, SAMe contributes to histone, DNA and RNA methylation and therefore to epigenetic regulation of gene expression. SAMe is also an important cofactor for the second step of catechol-o-methyl transferase (COMT) metabolism of catecholamines and a cofactor for conversion of Noradrnaline (NA) to Adrenaline (AD). At the bottom of the methylation cycle, the vitamin B6-dependant transsulfuration pathway is connected to the methionine cycle through homocysteine, leading to the generation of cysteine and eventually glutathione, one of the major redox-regulating agents in cells. In addition, interactions between free copper, vitamin B6, catecholamine synthesis and glutathione synthesis pathways have been outlined in the literature Enzymes: BHMT—Betainehomocysteine methyltrasferase, COMT—catechol-o-methyl-transferase, CBS—Cystathione Beta Synthetase, MAT—Methionine adenosyltransferase, MTHFR—Methylenetetrahydrofolate reductase, SAMe—S-denosylmethionine, MT—Methyltransferase, SAHH—S-Adenosylhomocysteine-hydrolase, MSR—Methionine sulphoxide reductase, MS—Methionine synthase. Vitamin cofactors: vitamin B6 (pyridoxine), vitamin B12 (cobalamin), vitamin C, folic acid, 5 methyl tetrahydrofolate. Mineral enzyme cofactors: Free (unbound) copper (Cu), zinc. Intermediate substrates: BH4—tetrahydrobiopterin BH2—dihydrobiopterin, DMG—Dimethylglycine, DOPAL—dihydroxyphenylacetaldehyde, DOPAC—dihydroxyphenylacetic acid, DOPEGAL—dihydroxyphenylglycolaldehyde, DOMA—dihydroxymandelic acid DHPG—dihydroxyphenylglycal, DOPA—dihydroxyphenylalanine, FAD—flavin adenine dinucleotide, 5HIAA—5-hydroxyondolacetic acid, HVA—homovanillic acid, MAO—monoamineoxidase, MHMA—3-methoxy-4-hydroxymandelic acid, MHPG—4-hydroxy-3-methoxyphenylglycol, SAH—S-adenosylhomocysteine, TMG—Trimethylglycine, VMA—Vanillylmandelic acid. HPL Urinary hydroxyhemopyrroline-2-one.

FIG. 2. Graph showing correlative strengths and relationships for vitamin B2 levels between MTHFR 677 TT, CT and CC variants with similar trajectory for P2-P1 and trajectory for creatinine as expected from glycine breakdown to creatine and creatinine being cofactored by SAMe.

FIG. 3. Graph showing elevated case-correlative strength and relationships for urine riboflavin (B2) and (P2) and HPL in the MTHFR 677 TT variant. In contrast, reduced case-correlative strength for urine riboflavin (vitamin B2=Peak 2) in wild type MTHFR 677 CC variant.

FIG. 4. Graph showing elevated case-correlative strengths and relationships for vitamin B2, B6 and D in the MTHFR 677 TT variant and variable folate strength between MTHFR 677 TT, CT and CC variants.

FIG. 5. Graph showing correlative strengths and relationships of vitamin B2 and P2-P1, P1-P2 and HPL/SG between MTHFR 677 TT, CT and CC variants.

FIG. 6. Graph showing elevated correlative strength of 5HIAA excretion in the MTHFR 677 CC variant and the correlative relationship of 5HIAA between MTHFR 677 TT, CT and CC variants.

FIG. 7. Graph showing elevated correlative strength for vitamin B12, HPL/SG and % free Cu/Zn and low correlative strength of zinc in the MTHFR 677 TT variant and relationships of vitamin B12, HPL/SG, zinc and the ratio of % free copper to zinc between MTHFR 677 TT, CT and CC variants.

FIG. 8. Graph showing correlative relationships of creatinine, vitamin B2/creatinine ratio, P2-P1/creatinine ratio, P1-P2/creatinine ratio and HPL/creatinine ratio between MTHFR 677 TT, CT and CC variants.

### DETAILED DESCRIPTION OF THE INVENTION

Unless defined otherwise, all technical and scientific terms used herein have the same meaning as commonly understood by those of ordinary skill in the art to which the disclosure belongs. Although any methods and materials similar or equivalent to those described herein can be used in the practice or testing of the present disclosure, typical methods and materials are described.

The articles “a” and “an” are used herein to refer to one or to more than one (i.e., to at least one) of the grammatical object of the article. By way of example, “an element” means one element or more than one element.

Throughout this specification and the claims which follow, unless the context requires otherwise, the word “comprise”, and variations such as “comprises” or “comprising”, will be understood to imply the inclusion of a stated element, integer or step, or group of elements, integers or steps, but not the exclusion of any other element, integer or step, or group of elements, integers or steps.

As used herein, “genotype” refers to the diploid combination of alleles for a given genetic polymorphism. A homozygous subject carries two copies of the same allele and a heterozygous subject carries two different alleles.

In the context of this specification, the term “MTHFR gene” refers to a gene coding for methyltetrahydrofolate reductase (see Goyette et al. (1994) Nat Gen 7:195-200). The term “MTHFR gene” as used herein refers to the human MTHFR gene, the nucleotide sequence of which is provided as NCBI Reference Sequence NM_005957 at http://www.ncbi.nlm.nih.gov/nuccore/NM_005957, the disclosure of which is incorporated herein by reference. A polymorphism at position 677 of the nucleotide sequence of the MTHFR gene, wherein cytosine may be replaced by thymidine (herein the “MTHFR C677T polymorphism” or “MTHFR C677T variant” (see Hustad et al. (2007) Am J Hum Gen 80:546-855), results in the replacement of an alanine residue with a valine residue at codon 222 of the protein. The genetic polymorphism is denoted “C677T”, where the number refers to the position of the polymorphisms with respect to the nucleotide sequence; the “C” is the nucleotide present in the reference or wild type sequence; and the “T” is the nucleotide residue present at that position in the variant sequence. Thus, the term “CC genotype”, “wild type” or “MTHFR 677 CC” variant or polymorphism herein refers to the presence of the C nucleotide at the 677 polymorphic site in both alleles, the term “CT genotype”, “heterozygote” or “MTHFR 677 CT” variant or polymorphism refers to the presence of the C nucleotide at the 677 polymorphic site of one allele and the T nucleotide at the 677 polymorphic site of the other allele. The term “TT genotype”, “homozygote” or “MTHFR 677 TT” variant or polymorphism herein refers to the presence of the T nucleotide at the 677 polymorphic site in both alleles. The encoded enzyme of the TT genotype has thermolability and weaker activity with weaker affinity for its FAD cofactor and impaired ability to manufacture sufficient 5 MTHF to supply the methylation cycle. For the purposes of the present invention, determination of which MTHFR allele is present in an individual may be referred to as determining the genotype, or the MTHFR genotype of the individual. In relation to MTHFR, the term “gene” and “allele” may be used interchangeably herein.

As used herein the term “vitamin” is understood to include any of various fat-soluble or water-soluble organic substances including but not limited to vitamin A, Vitamin B1 (thiamine), Vitamin B2 (riboflavin), Vitamin B3 (niacin or niacinamide), Vitamin B5 (pantothenic acid), Vitamin B6 (pyridoxine, pyridoxal, or pyridoxamine, or pyridoxine hydrochloride), Vitamin B7 (biotin), Vitamin B9 (folic acid), and Vitamin B12 (various cobalamins; commonly cyanocobalamin in vitamin supplements), vitamin C, vitamin D, vitamin E, vitamin K, K1 and K2 (i.e. MK-4, MK-7) that are essential in minute amounts for normal cellular activity.

Herein the term “catecholamines” refers to the group of monoamines that comprise adrenaline (AD), noradrenaline (NA) and dopamine (DA). The term “indolamines” refers to monoamine neurotransmitters comprising serotonin and melatonin.

As used herein, the term “psychosis phenotype” or “psychosis subtype” refers to a group of genetic and biochemical features that are characteristic of a subset of individuals with psychosis. The term “psychosis phenotype” can also refer to a characteristic biochemical and/or sensory processing profile. The present invention characterizes two major psychosis phenotypes. The first psychosis phenotype contemplated by the present invention is an under-methylating psychosis phenotype characterized by wildtype (CC) genotype at position 677 of the MTHFR gene or the heterozygous (CT) genotype at position 677 of the MTHFR gene in the presence of high NA and/or DA and/or AD levels and/or a high AD/MHMA and/or high NA/MHMA ratio relative to control values, and/or low vitamin levels of vitamin B2, vitamin D, folate, vitamin B6 and/or high levels of vitamin B12 relative to control values. The second psychosis phenotype of the invention comprises the homozygous (TT) genotype at position 677 of the MTHFR gene characterized by an absence of elevated levels of NA/MHMA and/or AD/MHMA relative to control values, and/or normal or elevated levels of vitamin B2, vitamin D, folate, vitamin B6 and/or low 5HIAA, and/or high % free copper/zinc ratio relative to control values is generally indicative of a low MTHFR enzyme activity, and presents a low MTHFR enzyme activity, over-methylating psychosis phenotype.

The term “control” or “control sample” as used herein refers to one or more biological samples from individuals or groups of individuals classified as not having schizophrenia or psychosis and where the diagnosis for the “control” or “control sample” has been confirmed. A “control sample” may comprise the compilation of data from one or more individuals whose diagnosis as a “control” for the purposes of the present invention has been confirmed. That is, for the purposes of practicing embodiments of the present invention samples to be used as controls need not be specifically or immediately obtained for the purpose of comparison with the sample(s) obtained from the subject under assessment. “Control values” may comprise the measured value or ROC value of a marker as measured in a control sample. “Values” of biomarkers and variables from subjects or controls refer to continuous values or ROC values for the biomarkers and variables.

As used herein the terms “treating”, “treatment”, “preventing” and “prevention” refer to any and all uses which remedy a condition or symptom, prevent the establishment of a condition or symptom, otherwise prevent, hinder, retard, or reverse the progression of a condition or symptom in any way whatsoever, or improve a condition or symptom. Thus the terms “treating” and “preventing” and the like are to be considered in their broadest context. For example, treatment does not necessarily imply that the subject is treated until total recovery. In conditions which display or are characterized by multiple symptoms, the treatment or prevention need not necessarily remedy, prevent, hinder, retard, or reverse all of said symptoms, but may prevent, hinder, retard, or reverse one or more of said symptoms.

As used herein, the term “effective amount” refers to an amount of an active agent, such as riboflavin, that is sufficient to effect one or more beneficial or desired outcomes. An “effective amount” can be provided in one or more administrations. The exact amount required will vary depending on factors such as the subject being treated, the age and general health of the subject, and the form in which the composition is administered. Thus, it is not possible to specify an exact “effective amount”. However, for any given case, an appropriate “effective amount” may be determined by one of ordinary skill in the art using only routine experimentation.

As used herein, the following terms may have, in particular contexts, the meanings ascribed:
metabolic signatures=particular groups of any one or more biological markers associated with, linked to and/or predicted to be associated with a particular MTHFR 677 variant;
sensory processing deficits=any one or more biological markers associated with finding of abnormal markers and/or measures on external assessment of vision or hearing and/or assessment of intracerebral, visual or auditory processing;
symptom=reported or observed feeling, perception, behavior, reported abnormality, absence of expression or behavior and/or inordinately behavior expression, experience or feeling that is different from normal internal or external experience,
risk factor=a factor in an individual's experience, feeling, perception, clinical examination, personal or family history and/or environment, established within epidemiological literature as associated with higher risk of onset of and/or association with psychosis, schizophrenia and/or schizoaffective disorder, such as, but not confined to, history of developmental disorder and/or significant delay, history of learning disorder, history of ear infection, history clinical and/or sub-clinical head injury, history of mental illness, and/or abnormality on otoscopy examination;
Functional Outcome Measures=measures and/or indices or markers of an individual's function as gauged by level of, for example, symptom intensity rating (SIR), global assessment of function (GAF), hospital admission frequency, disability pension (DSP) requirement, clinical global impression (CGI) of illness severity, social and occupational functioning scale (SOFAS) measure, and/or duration of illness (DOI);
vitamins=ingested or synthesized specialized compounds that are either essential or non-essential for activity of body biological systems and frequently act as cofactors for enzymes in body biochemistry.
intermediate substances=substrates and/or products of enzyme reactions or spontaneous reactions or conversions of substances in the chain of biochemistry pathways as described herein. Examples include but are not confined to histamine and homocysteine;
cofactors=vitamins, trace elements, metals and/or proteins that facilitate enzyme reactions;
psychosis progression=increased number, value, level, measure, intensity of symptoms, increase and/or decrease of functional outcomes measures and/or increase and/or decrease of biological markers, occurring in the course of development of psychosis from first episode symptoms and/or mental state;
psychosis relapse=regression to a more abnormal mental state, increase in number or intensity of symptoms of psychosis and/or deterioration of functional outcome or perception of increased illness severity;
treatment resistance=a mental state and/or collection of symptoms of psychosis which are not amenable, or are poorly amenable, to improvement by treatment or management or intervention measures.

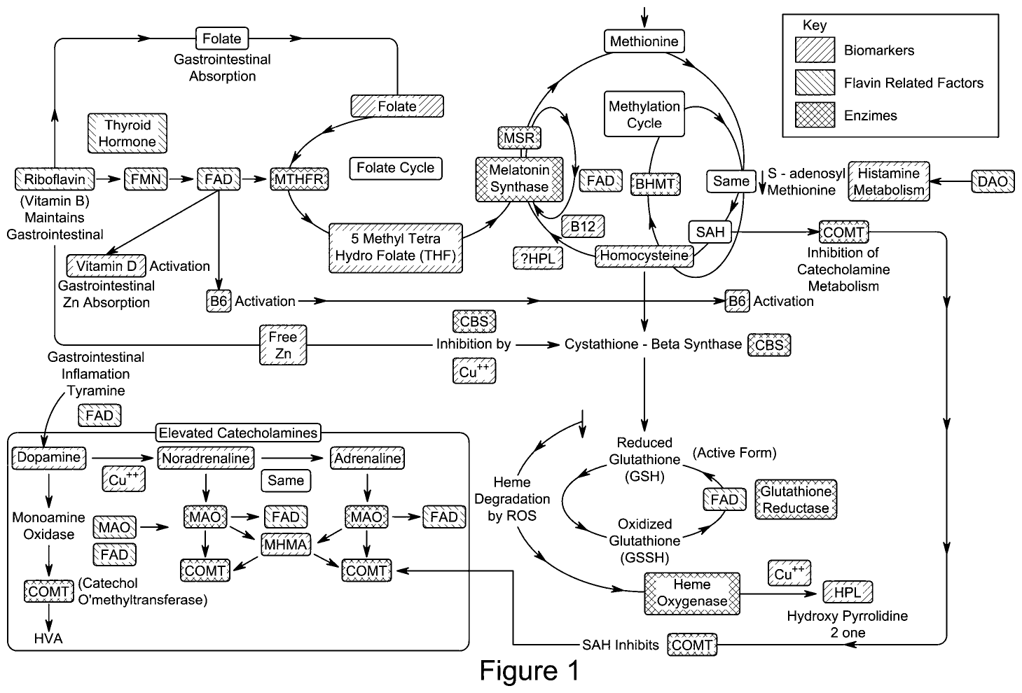

Throughout this specification, the following abbreviations are sued, in addition to those detailed in relation to FIG. 1:
ASOP % age diff—Speed of auditory processing with respect to age
CW % age difference—dichotic listening capacity with respect to norm for age
GSH reduced (active) glutathione
GSSH oxidised form of glutathione
HCY homocysteine
HPL hydroxypyrroline-2-one
HPL/SG hydroxy pyrroline-2-one in relationship to urine specific gravity
Reverse digit span—auditory working memory test
SAMe S-adenosyl homocysteine
SHMT serine hydroxymethyl transferase
Visual span—visuospatial working memory test
Vitamin B6. Pyridoxine 5 phosphate
Vitamin B12. Cobalamin
VSOP age add %—Speed of visual processing with respect to age
Vit=vitamin
Soc=SOFAS
zn=zinc,
free cu=% free Copper
vsop age add=visual speed of processing (age addition)
adna_mhma=[AD+NA]/MHMA
asopagediff=auditory speed of processing (age difference)
/=divided by
X=multiplied by
ALGORITHM*=algorithm risk prediction available
PPV=positive predictive value
NPV=negative predictive value.

As exemplified herein, the inventors have identified three phenotypes in sufferers of schizophrenia and schizoaffective disorders based on their genotype at the MTHFR 677 allele (referred to herein as “MTHFR 677 variant” as defined above). The MTHFR 677 allele was selected as a discriminator in this invention as contradictory findings have been reported in the literature for the role of this allele in schizophrenia, depression and bipolar disorder, when data has not been gene-variant-differentiated. Using the three MTHFR 677 genotypic variants (CC, CT and TT), embodiments of the present invention demonstrate that these variants play an important pilot role in determining schizophrenia and schizoaffective disorders and psychosis endophenotypes, with potential to also explain different forms of bipolar disorder.

### Operative Biochemical Pathways

A complete understanding of the various biomarkers described in the present disclosure and the interplay between them benefits from an understanding of the relevant underlying biochemical pathways. FIG. 1 presents the basic layout of interactive biochemical pathways of relevance to the MTHFR 677 C->T genotype. In a bicyclic process referred to as one-carbon metabolism, the folate cycle is coupled to the methionine (methylation) cycle through the generation of 5-methyl-tetrahydrofolat (activated folate—5MTHF) by the flavin dependent enzyme methyl tetrahydrofolate reductase (MTHFR). Protein for this enzyme is coded by the MTHFR 677 C->T gene where Cytosine may be replaced by Thymidine at the 677 position. Understanding the further role of riboflavin (vitamin B2) and flavin proteins (flavin adenine nucleotide, FAD and its precursor flavin mononucleotide, FMN), in psychosis progression is important. FAD and its immediate precursor FMN, which is derived directly from riboflavin (vitamin B2) which is obtained from dietary intake and reported synthesised by gastrointestinal organisms such as Lactobacilli, Escherichia coli, Bacillus subtilis and Saccharomyces cerevisiae. FMN and FAD are necessary for the activation or reconstitution of several vitamins related to the folate, methylation and catecholamine pathways. These vitamins are vitamin D, vitamin B6 and vitamin B12.

The MTHFR enzyme requires FAD as a cofactor and its activity is weakened without this cofactor availability. FAD is synthesised from Flavin mononucleotide (FMN) by the enzyme FAD synthase, whereas FMN is synthesised directly from riboflavin (vitami B2) by the thyroid and cortisol sensitive enxyme flavin kinase. FAD also cofactors the monoamine oxidase (MAO) enzyme which metabolizes the first step of catecholamine-metabolism. FAD also acts as a cofactor for the MTHFR enzyme which converts folate into its activated form (5 methyl tetrahydrofolate—5-MTHF). Therefore, when FAD is unavailable, catecholamines are elevated with respect to their metabolites (high NA/MHMA and AD/MHMA) and there is low synthesis of the major body methylator, S-adenosyl methionine (SAMe) from its precursor amino acid methionine. In addition, FAD is a necessary cofactor for the reconversion of oxidized glutathione (GSSH) back to its active, reduced form (GSH). Oxidative stress is a state of excessive free radical formation, which is a key component of schizophrenia. FMN is required to restore vitamin B12 function after its cofactoring of Methionine Synthase (MS) enzyme at the critical junction point between the folate and methionine cycles. Importantly, riboflavin (vitamin B2) is required for activation of vitamin B6, without which activated B6 cannot perform its role as a cofactor for a variety of strategic enzyme reactions.

The Methylation, Folate and Biopterin Cycles, constitute an inter-linked circular framework (see FIG. 1). On the left-hand side of FIG. 1, the synthesis of serotonin and dopamine, from precursors, tryptophan and tyrosine, respectively, is cofactored by vitamin B6. On the right hand side of FIG. 1, a major methylating enzyme methionine adenosyltransferase (MAT) is seen together with its products, S-adenosyl methionine (SAMe) and downstream S-adenosylhomocysteine (SAH). SAMe is a major source of methyl groups required for homocysteine synthesis, catecholamine-metabolism and histamine-metabolism. As a major methyl donor in cells, SAMe contributes to hi stone, DNA and RNA methylation and therefore to epigenetic regulation of gene expression

The enzyme methyltetrahydrofolate resuctase (MTHFR), occupies a pivotal position at a juxtaposition point between the folate and methylation cycles. Via its effect on the the biopterin cycle, this enzyme and its product 5 methyl tetrahydrofolic acid facilitates downstream (left-hand) catecholamine-synthesis of dopamine (DA) and serotonin, respectively, but also has upstream effects via SAMe's cofactoring of catechol-o methyl transferase (COMT) enzyme that allows catecholamines to be metabolised. MTHFR enzyme's product, 5-methyl tetrahydrofolate 5-(MTHF) is a necessary cofactor component for methionine synthase (MS) enzyme to reconstitute methionine from homocysteine, thus running the methylation cycle (also called the one-carbon cycle) and when its absence leads to reduced reconstitution of methionine and reduced flow on SAMe synthsis, this status is referred to as “under-methylation”.

When there is insufficient riboflavin absorbed and therfore insufficient flavoprotein (FAD) product to co factor the MTHFR enzyme, an insufficiency of 5-MTHF product to run the methylation cycle results. This deficit results in relatively low-output of S-adenosyl-methionine (SAMe)[, which serves as an essential methyl-donor for catecholamine metabolism, by catechol-o-methyl-transferase (COMT). This means that catecholamines are conserved (elevated). Catechol elevation may occur to the extent that they must be solely metabolised by monoamine oxidase after which, they are further metabolised by break-away metabolic pathways that release neurotoxic, neuro-degenerative final products. However in a setting of insufficient FAD to cofactor Monoamine Oxidase (MAO), in activity of this enzyme also leads to upstream trapping of catecholamines. Since SAMe also serves as a cofactor for adrenalin (AD) synthesis from noradrenalin (NA) and as a methyl-donor for histamine metabolism by N-methyl-histaminase, NA is trapped and its levels exceed those of AD (NA/AD is elevated. In such a setting of low methylation, with low SAMe output from the methylation cycle, histamine may also accumulate, because it requires SAME availability as a co-factor for its metabolism.

Copper (Cu) is also bound in serum by the protein ceruloplasmin (Cp), so a low ceruloplasmin level, results in high amounts of free-unbound copper in serum, which has neurotoxic consequences [24]. Copper and zinc compete for binding proteins and have an inverse homeostatic relationship within the body milieu. Copper inhibits DOPA-decarboxylase so cofactors noradrenaline synthesis from dopamine. It also an inhibitor of cystathione betasynthase enzyme (CBS), in the transulfuration pathway, which implicates it in oxidative-stress causation, since the end-product downstream of CBS, is glutathione, a major-brain-anti-oxidant. Vitamin B6 requires the flavoprotein FMN for its activation, therefore is disabled by insufficiency of riboflavin. Many enzymes along this homocysteine-metabolising, glutathione-synthesising, pathway, are also co-factored by vitamin B6 and blockage in this pathway causes backed-up homocysteine levels, that may be further-elevated in the presence of MTHFR-polymorphism, since this enzyme's product, 5MTHF is also required for homocysteine-metabolism.

5-MTHF is a particularly important product required for reconstitution of methionine, in the methylation cycle (FIG. 1). 5 MTHF is also a required to cofactor for homocysteine metabolism by the enzyme methionine synthase, explaining why its relative insufficiency is accompanied by notably high homocysteine levels in a setting where homocysteine's other metabolising enzyme cystathione beta synthase (CBS) may be inhibited by the high free copper. Lesser methionine reconstitution by 5-MTHFR unavailability also reduces SAMe formation to the extent that the “SAMe salvage pathway” (SSP) switches on and over-methylation commences. This SAMe salvage pathway opens up across the middle of the methylation cycle (FIG. 1), utilizing the zinc cofactored BHMT enzyme. Once this pathway activates it rapidly cycles SAMe round to SAH, which in turn replenishes homocysteine and so-on, around the cycle. Over time, use of the alternative SAMe salvage pathway for methylation results in zinc cofactor depletion. Because of the relationship of low zinc with high free copper, elevated free copper exerts its inhibitory effect on the activity of CBS, that lies beneath homocysteine at the top of the trans-sulfuration (TSF) pathway to glutathione synthesis (whereby 46% of homocysteine is usually metabolised). Therefore, in this MTHFR677 TT setting where the other MS pathways for 27% of homocysteine metabolism is also blocked by insufficient 5MTHFR product from the MTHFR enzyme, homocysteine metabolism is stopped and trapped homocysteine becomes very elevated, since its metabolism is being blocked by two routes. This occurs in a setting where homocysteine is still being synthesised by S adenosyl homocysteine methyl transferase (SHMT)—a B6-facilitated enzyme at the bottom of the methionine cycle. In both the homozygous MTHFR 677 TT setting of MTHFR unutilised FMN and FAD and the setting where riboflavin insufficiency for FMN and FAD synthesis compromises activity of the MTHFR enzyme, trapped homocysteine can load the betaine hydroxy-methyltransferase (BHMT) pathway, to an extent that it becomes over-active reaching a state known as “over-methylation”.

The pathways described as operating in this over-methylation dynamic are operative in the setting of the homozygous MTHFR 677 TT variant which has 5-10% prevalence in the Australian population. Because the MTHFR 677 TT allele codes for a thermolabile 75% inactive MTHFR enzyme, an important related understanding is that it's FAD cofactor is left somewhat unutilised by the inactive MTHFR enzyme and this unutilised FAD and FMN is therefore available for activation other enzymes and vitamin cofactors such as folate, vitamin B6 and vitamin D. Thus in a setting of strong positive case-correlates for vitamin B2, representing sufficient riboflavin bioavailability, as confirmed on urine analysis, strong positive correlates also exist for vitamin B6 and vitamin D are found in the TT phenotype. Thus, carriers of the TT variant have both plentiful FAD cofactoring the MAO enzyme and plentiful SAMe for cofactoring the COMT enzyme—both enzymes being responsible for catecholamine metabolism. This “double whammy” effect on catecholamine levels carries a risk of catecholamine depletion and severe depression. In this setting of high catecholamine synthesis and turnover, adrenal exhaustion is likely, with negative, features. Additionally, L tryptophan is channelled into the Kynurenic pathway to bolster levels of niacin that are required to mop up excess methyl groups as it cofactors conversion of SAMe to SAH. This explains why elevated 5HIAA is not a feature of the homozygous MTHFR 677 TT phenotype. In this setting folate remains low due to continued MTHFR enzyme incapacity, however, due to sufficiency of unutilised riboflavin, FMN and FAD levels are sufficient to activate vitamin B6 and vitamin D, so there are minimal sensory processing deficits with a considerably lesser number of psychosis symptoms. Despite this, the phenotypic expression of this variant can be deceptively simple, as it is associated with high levels of symptom intensity and high AD/NA ratio which means that fear-related paranoia together with suicidality and hostility may persist. In this phenotype there is also risk of adrenal exhaustion due to FAD facilitated elevated MAO-catecholamine metabolism which together with high free copper-induction of DA conversion to NA, can result in DA depletion with depressed mood and/or neurovegetative and/or negative features and/or suicidality.

Serine metabolism is expected to be halted in a low FMN-B6 inactivated, MTHFR CC setting where B6 is required for its metabolism to glycine. Serine catecholamine synthesis is trapped by insufficient activated B6. Therefore what serine is synthesised, may itself be trapped along with its product substance L tryptophan, which is similarly trapped and unable to be metabolized in the absence of FAD and B6 for tryptophan pyrrolase activity.

### Diagnosis and Prognosis

Described herein for the first time is the finding that different levels of vitamin cofactors vitamin D, vitamin B6, vitamin B12 and folate are associated with different methylation states and different MTHFR 677 genotypes or variants. Also described herein for the first time is the finding that riboflavin is conserved and elevated in the homozygous MTHFR 677 TT variant. Without wishing to be bound by theory, the inventors postulate that in this variant, riboflavin is underutilized due to inactivity of the thermolabile MTHFR enzyme coded for by the MTHFR 677 TT gene variant. For this reason, the surplus availability of vitamin B6 and flavin to cofactor methylation enzymes is linked to an over-methylation state with its own discrete metabolic signature and functional sequelae.

As described and exemplified herein, the inventors have developed novel methods for diagnosing schizophrenia and schizoaffective disorders and psychosis, based on the genotype at position 677 of the MTHFR gene. The inventors have carried out a statistical analysis of biochemical markers in sufferers of schizophrenia, schizoaffective disorders and psychosis, finding that FMN-FAD is 70% related to other variables in the data set including low levels of vitamin B6, vitamin D and folate. Accordingly, when methylation signatures subject are considered along with their MTHFR 677 genotype, levels of vitamins and indole-amines or catecholamines or intermediate substance(s) can be used singly and/or in ratios and/or in compound biomarker equations to diagnose a biological subtype and associated psychosis-related symptoms or the MTHFR 677 genotype itself can be used to provide an approximation of the likely underlying phenotype. Accordingly, methods described herein provide genetic and biochemical biomarkers that together, singly, in ratio and/or in variable combinations with each other indicate level of methylation or under-methylation related to gene variants and thereby possess clinical usefulness in management and diagnostic screening capacity for schizophrenia, schizoaffective disorder and psychosis, including schizophrenic and schizoaffective psychosis. The methods allow differentiation between under-methylating and over-methylating phenotypes of psychosis and will enable individualized, pathology-directed treatment.

As exemplified herein, the homozygous MTHFR 677 TT genotype coding for the poorly-functioning MTHFR enzyme is strongly related to elevated vitamin B2 as determined from urine sample values that may or may not be adjusted for creatinine. In such samples, there is also an elevated value for the ratio of Peak 2 amplitude and/or area/Peak 1 amplitude and or area and/or Peak 2 amplitude or area—Peak 1 amplitude or area, where Peak 2=riboflavin and Peak 1 represents excreted degradation products of riboflavin. These findings are linked to an over-methylation state and its metabolic signatures, sensory processing deficits, symptoms, risk factors and functional outcome measures.

Also as exemplified herein, the MTHFR 677 wild-type (CC) genotype coding for the normally-functioning MTHFR enzyme is associated with low levels of vitamins B6, vitamin D and folate and high vitamin B12, which are linked to an undermethylation state and its metabolic signatures, sensory processing deficits, symptoms, risk factors and functional outcome measures. The MTHFR 677 wild-type (CC) genotype is also associated with absence of a significant relationship with vitamin B2 as determined in urine samples, but an elevated value for the ratio of Peak 1 amplitude and/or area/Peak 2 amplitude and or area, where Peak 1 represents excreted degradation products of riboflavin and Peak 2=synthesized riboflavin. These findings are linked to an under-methylation state and metabolic signatures, sensory processing deficits, symptoms, risk factors and functional outcome measures.

In order to advantage longitudinal monitoring, long term management-planning, longer term prevention of psychosis progression and monitor progression towards chronic condition and/or permanent remission and/or cure of/ schizophrenia and/or schizoaffective psychosis and/or their symptoms of psychosis, as described herein the inventors have determined biomedical markers that predict extended duration of illness (DOI). Moreover, the inventors have developed predictors of various functional outcomes of schizophrenia, schizoaffective disorders and psychosis, as well as predictors or hostility and suicidality.

Methods described herein provide a combination of genetic and biochemical biomarkers that possess diagnostic screening capacity for schizophrenia, schizoaffective disorder and psychosis, including schizophrenic and schizoaffective psychosis. The method allows differentiation between an under-methylating phenotype, an over-methylating phenotype and a mixed methylation phenotype, correlating with the MTHFR 677 genotype, and enable individualized, pathology-directed treatment.

One aspect of the disclosure provides a method for diagnosing a psychosis phenotype in a subject with a psychotic disorder, the method comprising:
(a) obtaining one or more biological samples from said subject, and
(b) determining the status of the C677T polymorphism of the MTHFR gene from the one or more biological samples,
wherein
   (i) the presence of the homozygous CC genotype at position 677 of the MTHFR gene is indicative of an under-methylating psychosis phenotype,
   (ii) the presence of the homozygous TT genotype at position 677 of the MTHFR gene is indicative of a low activity MTHFR enzyme and an over-methylating psychosis phenotype, and
   (iii) the presence of the heterozygous CT genotype at position 677 of the MTHFR gene is indicative of a mixed-methylation psychosis phenotype.

Another aspect of the disclosure provides a method for diagnosing a psychotic disorder, optionally schizophrenia, schizoaffective disorder or psychosis, in a subject, the method comprising:
(a) obtaining one or more biological samples from said subject,
(b) determining the status of the C677T polymorphism of the MTHFR gene from the one or more biological samples,
wherein
   (i) the presence of the homozygous CC genotype at position 677 of the MTHFR gene is indicative of an under-methylating psychosis phenotype,
   (ii) the presence of the homozygous TT genotype at position 677 of the MTHFR gene is indicative of a low activity MTHFR enzyme and an over-methylating psychosis phenotype, and
   (iii) the presence of the heterozygous CT genotype at position 677 of the MTHFR gene is indicative of a mixed-methylation psychosis phenotype,
(c) determining levels of one or more biomarkers as defined in the first aspect, and optionally the ratios of selected biomarkers, and/or measuring or assessing one or more additional parameters as defined in the first aspect, and
(d) optionally comparing determined, measured or assessed levels, values or ratios from (c) with corresponding control levels, values or ratios from on or more individuals known not to suffer from the psychotic disorder.

The methods described herein typically comprise the determination of levels of one or more biomarkers, and optionally the ratios of selected biomarkers as described herein, in the one or more biological samples, to inform the diagnosis. Such biomarkers may, in particular embodiments, be selected from one or more of: free copper, zinc, indolamines and catecholamines and their metabolites, vitamin and mineral or trace element cofactors (such as vitamin D, vitamin B2 (riboflavin), vitamin B6, vitamin B12, folate), intermediate substances, and vitamin B2 excretion levels. In exemplary embodiments, the one or more biomarkers may be selected from: free copper, zinc, vitamin D, riboflavin (vitamin B2) and flavin-related compounds such as flavin adenine dinucleotide (FAD) and flavin mononucleotide (FMN), vitamin B6, vitamin B12, folate and related compounds, S-adenosylmethionine (SAMe), S-adenosylhomocysteine (SAH), hydroxylpyrolline-2-one (HPL), histamine, adrenaline (AD), noradrenaline (NA), dopamine (DA), 5-hydroxyondolacetic acid (5HIAA) and methylhydroxy vanillyl-mandelic acid (MHMA). The skilled addressee will appreciate that other biomarkers may also be measured. Exemplary additional biomarkers are detailed further below.

The methods described herein may also comprise the assessment or measurement of one or more additional parameters. Such parameters may include, but are not limited to, measurement or assessment of one or more symptom ratings for schizophrenia, schizoaffective disorder or psychosis, risk factor analysis, functional visual and auditory acuity, external ear canal patency, tympanic membrane status, motor capacity, extrapyramidal and thyroid status. Symptom ratings may be measured or assessed using one or more psychiatric symptom rating scales known to those skilled in the art. Exemplary symptom rating scales include, but are not limited to: the Brief Psychiatric Rating Scale (BPRS); the Positive and Negative Syndrome Scale (PANS S), the Global Assessment of Function (GAF) Scale; the Clinical Global Impressions (CGI) score; and the Social and Occupational Functioning Scale (SOFAS). An index for hospital admission frequency (number of admissions/DOI) and disability pension requirement (DSP) may also be determined. Exemplary risk factors for analysis include, but are not limited to, risk factors with regard history of ear infection, developmental disorder or delay, family history of mental illness, history of clinical or subclinical head injury, history of abuse, and history of learning disorder. The skilled addressee will appreciate that the above noted parameters, symptom ratings scales and risk factors are exemplary only. In some circumstances, typically depending on the subject in question, it may be appropriate to measure or assess one or more other suitable meters, symptom ratings scales and risk factors, either in addition to, or in place of one or more of those detailed above. Such additional meters, symptom ratings scales and risk factors will be well known to those skilled in the art.

Another aspect of the disclosure provides a method for diagnosing a psychotic disorder, optionally schizophrenia, schizoaffective disorder or psychosis, in a subject, the method comprising:
(a) obtaining one or more biological samples from said subject,
(b) determining levels of one or more biomarkers as defined in the first aspect, and optionally the ratios of selected biomarkers, and/or measuring or assessing one or more additional parameters as defined in the first aspect, and
(c) optionally comparing determined, measured or assessed levels, values or ratios from (b) with corresponding control levels, values or ratios from on or more individuals known not to suffer from the psychotic disorder,
wherein optionally the status of the C677T polymorphism of the MTHFR gene in the subject is known, or is determined.

Another aspect of the disclosure provides a method for predicting or determining expected duration of illness of a subject with a psychotic disorder, the method comprising:
(a) obtaining one or more biological samples from said subject,
(b) determining the status of the C677T polymorphism of the MTHFR gene and the psychosis phenotype according to the first aspect,
(c) determining levels of one or more biomarkers as defined in the first aspect, and optionally the ratios of selected biomarkers,
(d) measuring or assessing one or more additional parameters as defined in the first aspect, and
(e) determining expected duration of illness from an analysis of said determined, measured or assessed biomarkers and additional parameters.

Additional aspects provide for the prediction or determination of one or more additional functional outcomes or measures. Such functional outcomes or measures may be selected from: admission frequency; cost and/or care burden; requirement for disability support pension; symptom intensity rating (SIR); clinical global awareness of illness severity (CGI); global assessment of function (GAF) score; social and occupational function scale (SOFAS) rating value; hostility; and suicidality.

Genotype analysis and/or gene expression analysis used to determine the MTHFR 677 variant and biochemical tests used to determine biomarker levels in accordance with embodiments disclosed herein may be carried out utilising any means known in the art and the present invention is not limited by reference to the means by which the genotype or biomarker levels are determined. Determination of genotype and/or biomarker levels may comprise detection and/or quantitation and the methods and techniques available for such determination are well known to those skilled in the art.

Suitable methods and techniques for determining MTHFR 677 variant include, but are not limited to, hybridization-based methods, such as dynamic allele-specific hybridization, the use of molecular beacons or oligonucleotide single nucleotide polymorphism (SNP) arrays or microarrays; enzyme-based methods, such as restriction fragment length polymorphism (RFLP), PCR-based methods, the use of Flap endonuclease, primer extension, 5′-nuclease and oligonucleotide ligation assays; other post-amplification methods such as single strand confirmation polymorphism, temperature gradient gel electrophoresis, denaturing high performance liquid chromatography, high resolution melting of an amplicon, DNA mismatch-binding proteins, SNPlex or surveyor nuclease assay; sequencing or next generation sequencing.

Suitable methods and techniques of biomarker levels include, but are not limited to, the use of spectral analysis, column chromatography, gel electrophoresis, mass spectroscopy and identification of protein spots, enzyme-linked immunosorbent assay (ELISA), Western blot, photonic molecular sensing techniques, image acquisition and analysis (such as magnetic resonance imaging (MM) spectroscopy and single photon emission computed tomography (SPECT)) or other in-vivo imaging methods. Biochemical tests used to determine biomarker levels in accordance with embodiments disclosed herein may be employed in any suitable environment or setting, such as a hospital, clinic, surgical or medical practice, or pathology laboratory. Alternatively, or in addition, such biochemical tests may be incorporated into one or more devices capable of analysing the desired biomarkers to thereby allow a degree, or complete, automation of the testing process. Suitable devices are typically capable of receiving a biological sample, analysing one or more biomarker levels in said sample and providing data on said biomarker level(s) in real time thus facilitating bench-to-bedside and point-of-care analysis, diagnosis, risk assessment and/or treatment. Suitable devices include, but are not limited to, the Cobas® in vitro diagnostic systems (Roche Diagnostics). The device may be a handheld device or an assay device containing micro-chip technology.

Diagnoses and risk predictions made in accordance with embodiments disclosed herein may be correlated with or determined in conjunction with conventional diagnoses, for example as generally exemplified by the International Diagnostic and Statistical Manual of Mental Disorders, Fourth Edition, (DSM IV or DSM IV-R) (the disclosure of which is incorporated herein by reference in its entirety) or DSM V or other international mental disease or symptom classification systems known to those skilled in the art.

The symptoms of psychotic disorders are diverse, encompassing almost every aspect of cognition and behaviour and may be characterized as positive or negative symptoms. Positive symptoms are those that are brought on by the disorder (e.g. hallucinations, delusions). Negative symptoms are those qualities taken away by the illness (e.g. one's drive and motivation are gone). These symptoms may be scored according to the Positive and Negative Syndrome Scale (PANSS). PANSS is a medical scale used for measuring symptom severity of patients with schizophrenia. It is widely used in the study of antipsychotic therapy. The name refers to the two types of symptoms in schizophrenia, as defined by the American Psychiatric Association: positive symptoms, which refer to an excess or distortion of normal functions, and negative symptoms, which represent a diminution or loss of normal functions.

Accordingly, methods of the present invention may further include assessment and/or monitoring in subjects of one or more symptoms associated with schizophrenia, schizoaffective disorder and/or psychosis. Without limiting the scope of the present disclosure, exemplary symptoms may include somatic concern, anxiety, depressed mood, suicidality, guilt, hostility, aggression, elated mood, grandiosity, pressure of speech, suspiciousness/persecution, auditory or visual hallucinations, ideas of reference or control, unusual or bizarre thought content, loose associations of thought, thought disorder, bizarre behaviour, self-neglect, self-harm, threats to others, disorientation, conceptual disorganisation, blunted or flat affect, emotional withdrawal, apathy, social withdrawal, social anxiety, motor retardation, tension, uncooperativeness, excitement, inattention, distractibility, motor hyperactivity, mannerisms or posturing, movement disorder, delusions, poor rapport, passivity, poor abstract thinking, reduced or absent theory of mind, reduced insight, reduced judgement, reduced short or long-term memory, anti-social traits, tendencies or acts, chronic regional pain or other unexplained chronic pain syndrome, offending behaviour of a forensic nature, disturbance of volition, poor impulse control, anger, delayed gratification difficulty, affective-lability, mood lability, mood swings, manic depression mood shifts, active social avoidance, preoccupation, obsessional preoccupation, ruminations, disturbance of spontaneity or flow of conversation, poor self care, anxious worrying, tension, tonicity, grasp strength, anxious rumination, fear, active/intentional and passive/unintentional avoidance, dissociation, stress, attenuated psychotic symptoms, overvalued ideation, brief intermittent psychotic symptoms, subjective self-disturbance, re-experiencing phenomena, sense of presence, distancing, corporeality, disturbed stream of consciousness, self-other boundary disturbances, self-demarcation disturbances, body-image disturbances, anorexia, orientation and re-orientation disturbances, self-consciousness, first rank passivity symptoms, ideas of reference or control, loss of sense of self, thought insertion, thought broadcasting, thought blocking, thought replacement, abnormal perception, delusional attribution or interpretation, under-arousal, disinhibition, impulsivity, over-arousal, difficulty attending, reduced attention span, scattered attention, distressing recollections, emotional dysregulation, implausible belief, obsessional thought-preoccupation or thoughts, compensations, intrusive auditory thoughts, euphoria, apathy, irritability and/or poor impulse control.

In accordance with embodiments of the present disclosure, suitable ratings scales, well known to those skilled in the art, that may be employed include the Brief Psychiatric Rating Scale (BPRS), the Positive and Negative Syndrome Scale (PANS S), the Global Assessment of Function (GAF) Scale, the Clinical Global Impressions (CGI) score, the Social and Occupational Functioning Scale (SOFAS), the Abnormal Involuntary Movement Scale (AIMS), the Behaviour and Symptom Identification Scale (BASIS-32) and the revised 24 item scale (BASIS-24), the Clinician Administered PTSD Scale (CAPS), the Depression Anxiety Stress Scales (DASS), the Dissociative Experiences Scale (DES), the Yale-Brown Obsessive Compulsive Scale (Y-BOCS), the Young Mania Ratings Scale (YMRS), the Comprehensive Assessment of At-Risk Mental States (CAARMS), the At-Risk Mental State for Children and Adolescents (ARMS), the Structured Interview of Psychosis-risk Syndromes (SIPS), and the Attenuated Psychotic Symptom Syndrome (APSS). Herein, the term, “disease severity” may refer to severity defined by clinical global impression of severity (CGI) and Symptom intensity rating (SIR).

In diagnosing schizophrenia, schizoaffective disorder and psychosis, including schizophrenic and schizoaffective psychosis, and psychosis phenotype, and predicting association with schizophrenia or risk of an individual developing schizophrenia, schizoaffective disorder or psychosis, including schizophrenic and schizoaffective psychosis, in accordance with embodiments disclosed herein, determination of markers as disclosed herein may be used in conjunction with a range of other sensory-based, cognitive and behavioural tests known and available to those skilled in the art including, for example, Go-NO-GO test, digit-symbol processing speed and accuracy test, an acoustic reflex and reflex decay test; anxiety potentiated startle reflex; startle reaction time; acoustic startle (threshold, inhibition and affective inhibition); auditory brain stem responses (ABR) such as stimulus threshold, waveform morphologies, absolute and relative amplitudes, latencies, middle latency response (MLR) and relative interpeak latencies for ABR waves N1, Na, Pa, Pb and late latency response (LLR), N1, P2 and P3 (P300) components, auditory tone (pitch) discrimination test, division of auditory attention test, filtered word test, auditory figure ground test, visual field evoked response test, prepulse inhibition test, quantitative EEG and topographic mapping of alpha, beta, theta and delta waves and all possible power ratios between these waves, including absolute power, relative power and power relative to normal data base, spectral analysis, independent component analysis, Z score analysis and signal source analysis; visual response search score; eye blink rate; mismatch negativity; auditory (and visual) evoked response potentials and the P 50, N1, P1, N2, P200, P250 and P300 components of the evoked response and their amplitude laterality discrepancies and interpeak latencies, retrograde memory; immediate memory; memory selection; executive function; N-back test; response speed; directed ignoring task; go/no go response inhibition; internal/external locus of control; strength of memory score; memory tests (e.g. Ray copy/recall, RAVLT and RAVLT errors, SILS, quick T, IT); saccadic eye movements; antisaccade task; EEG gamma band synchrony; and auditory (and visual) evoked response tests, components including mismatch negativity component (MMN), N1, P50, P400, P3a and P3b components during a cognitive task, contingent negative variation component (CNV) and post-imperative negative variation (PINV) component Auditory Brain stem Response (ABR) stimulus threshold, waveform morphologies, absolute and relative amplitudes, latencies, middle latency response (MLR) and relative interpeak latencies for ABR waves N1, Na, Pa, Pb and late latency response (LLR), N1, P2 and P3 (P300) components, ABR frequency and amplitude laterality differences, ABR interpeak latencies, frequency and power analysis of BOLD fMRT signal for sensory, motor, cognitive or integrated tasks and/or brain networks.

In particular embodiments of the present disclosure, analysis of biomarker levels, values or ratios are subjected to one or more statistical analyses. For example, in particular embodiments, methods of the present disclosure comprise conducting one or more statistical analyses of determined values of markers derived from a subject, in combination, and diagnosing schizophrenia, schizoaffective disorder or psychosis in the subject on the basis of the combined analysis. Such statistical analyses may include receiver operating characteristic (ROC) analysis, logistical regression analysis, Spearman's rank-order correlation analysis and the Mann-Whitney U test. ROC analysis may comprise determining ROC ranges for individual ROC variables, cut off limits, compound ROC variables and/or continuous variables, equations involving ROC variables and/or continuous variables. Suitable statistical analyses and methods for carrying them out will be well known to those skilled in the art.

Methods of diagnosis in accordance with the present invention may include preliminary assessment tests and demographic, symptom and functional assessments in conjunction with the MTHFR gene variant methylation status-guided diagnosis and assessments described and exemplified herein.

Exemplary preliminary assessment tests include: determining values for, or levels of, any or all of one or more risk factor markers including, but not confined to risk factors such as a history of developmental disorder and/or delay, history of learning disorder, history of ear infection, family of mental illness, and/or clinical or subclinical injury; determining duration of illness (DOI); determining normality or abnormality of hearing status and of the external ear drum as detected on clinical otoscopy examination and comparing the two findings; determining normality or abnormality of visual status in terms of short and/or long vision acuity and/or cover test to exclude phoria; clinical examination to exclude of extrapyramidal side effects in head, arms, hands or neck; determining normality or abnormality of motor function with respect to capacity to physically perform motor responses in physiological sensory processing assessment; determining normality or abnormality of thyroid function; and/or determining substance use and prescribed and unprescribed medication and supplement status.

Methods in accordance with embodiments of the disclosure may include one or more of the following determinations or assessments in addition to determining the MTHFR 677 genotype of an individual:
Determine values for, or levels of % free copper and zinc and/or % free copper to zinc ratio, in one or more biological samples and comparing these relative to values, levels or ratios typical of that individual's determined MTHFR 677 genotype and comparing values, levels or ratios with control levels/ratios for subjects not suffering from schizophrenia or schizoaffective disorder or psychosis, and/or comparing these values, levels or ratios with prediction markers for case-ness and/or risk of adverse functional outcome measures for the individual's MTHFR 677 genotype.
Determine values for, or levels of, one or more indolamines or catecholamines and/or their metabolites, including ratios thereof, in one or more biological samples and comparing values, levels or ratios to control values, levels or ratios in subjects not suffering from schizophrenia, schizoaffective disorder or psychosis, wherein the one or more indolamines or catecholamines and/or their metabolites include noradrenaline (NA), adrenaline (AD), dopamine (DA) methylhydroxyymandelic acid (MHMA), serotonin, and/or 5HIAA.
Determine values for, or levels of, one or more vitamins, including ratios thereof, in one or more biological samples and comparing values, levels or ratios to control values, levels or ratios in subjects not suffering from schizophrenia, schizoaffective disorder or psychosis, wherein the one or more vitamins include folate, vitamin B6, vitamin B12 and/or vitamin D.
Determine values for, or levels of, one or more markers of biochemical intermediate substances such as homocysteine and/or histamine including ratios thereof, in one or more biological samples, and comparing values, levels or ratios to control values, levels or ratios in subjects not suffering from schizophrenia, schizoaffective disorder or psychosis.
Determine values for, or levels of, excretion of HPL/SG and/or HPL/creatinine.
Determine values for, or levels of, vitamin B2 either in amount as ug/L and/or represented as amplitude and/or area under HPLC elution Peak 2 (P2), and/or amplitude and/or area under elution Peak 1 (P1), in ratio P1/P2 and/or P2/P1 and or subtracted from each other (P2-P1 and or P1-P2), where peak 1 represents riboflavin related products and/or peak 2 is riboflavin.
Determine auditory and visual sensory processing measures.

In exemplary embodiments, methods of diagnosis according to the present disclosure and determination of, for example, a psychosis risk index for an individual, may comprise one or more of the steps, assessments or determinations set out below:
Determine individual demographics, substance use, medication status, and DOI.
Determine risk factor status with regard to history of ear infection developmental disorder and/or delay, family history of mental illness, history of clinical and/or subclinical head injury, history of abuse, history of learning disorder.
Determine individual functional visual and auditory acuity, external ear canal patency, tympanic membrane status, motor capacity, extrapyramidal and thyroid status.
Determine symptom rating symptom frequency and intensity on Brief Psychiatric Rating Scale (BPRS).
Determine DOI and functional outcome rating measures for SIR, GAF, CGI, SOFAS, suicidality, hostility or any other particular symptom of interest required.
Determine individual values for, levels of, or ratios for any or markers selected from, for example, % free copper, zinc, indolamines and catecholamines and their metabolites, vitamin and mineral or trace element cofactors, intermediate substances, HPL/SG and/or HPL/creatinine, vitamin B2 excretion levels and/or Peak 1 and 2 ratios or subtractions for amplitude and/or area under the peak, in one or more biological samples.

Methods may thereafter comprise comparing individual levels, values or ratios relative to ROC cut-off levels, values, ratios typical of control levels, values or ratios for subjects, optionally in the same population, who are not suffering from schizophrenia or schizoaffective disorder or psychosis, to determine the number of abnormal variables, relative to controls with respect to cut-off values, and from this number, determine a diagnostic psychosis risk index relative to controls and note type of abnormal variables relative to controls who do not suffer from schizophrenia or a schizoaffective disorder or psychosis.

Methods may also comprise comparing logistic regression prediction analysis on individual levels, values or ratios against values, levels or ratios of normal controls, optionally derived from the same population area, of the same MTHFR 677 genotype, to determine the number of abnormal variables, relative to controls who do not suffer from schizophrenia or a schizoaffective disorder or psychosis. From this number, one can determine a diagnostic risk index relative to controls and note type of abnormal variables compared with controls.

Methods may also comprise comparing individual measures or values of visual and/or auditory processing relative to ROC measures, values, percentage cut-off levels or values or percentages typical of control subjects, optionally in the same population area, of the same MTHFR 677 genotype, who do not suffer from schizophrenia or a schizoaffective disorder or psychosis. From this comparison, one can determine the number of abnormal variables, relative to controls and from this number determine a diagnostic psychosis risk index relative to controls and note type of abnormal variables compared with controls.

Methods may also comprise determining individual measures, values, levels, percentages as discussed above relative to ROC measures, values, levels, percentage cut-off measures or values or percentages typical of control subjects, optionally in the same population area, of the same MTHFR 677 genotype who do not suffer from schizophrenia or a schizoaffective disorder or psychosis. From this comparison, once can determine the number of abnormal variables, relative to controls and from this number determine a composite diagnostic psychosis risk index relating to both biomedical and sensory processing variables and note type of abnormal variables compared with controls. By further logistic regression of ROC analysis, one can also determine the number of abnormal variables, relative to controls and from this number determine a threshold for diagnostic risk (i.e. a diagnostic risk index) relating to both biomedical and sensory processing variables and note type of abnormal variables compared with controls.

Methods may also comprise the inclusion of risk factors in determining the type and number of abnormal variables or measures and comparing these in combination with sensory processing variables and biomedical (or other) variables derived from assessments as described above, in order to derive a composite psychosis risk index, and noting type of abnormal variables. A stepped care and/or composite precision treatment and/or treatment programme may then be implemented for a subject, based upon single and/or sequential and/or composite remediation and/or treatment intervention(s) based on one or more abnormal marker types, levels, values, measures or ratios.

Clinical progress of the subject may be monitored over time, which monitoring may include one or more assessments, measurements or determinations as described above over time to assess, for example, remission, relapse, regression and/or rate of symptom frequency and/or intensity over time.

Methods of the disclosure may also comprise repeating measurement of functional outcome measures such as CGI, SIR, SOFAS, GAF and cost-care burden index (hospital admission frequency+DSP), one or more times during the course of, or following, treatment to compare values of these outcome measures over time and monitor progress of the subject, efficacy of the treatment and/or response of the subject to the treatment. Methods may also comprise repeating measurement of risk factors, BPRS and other symptom rating scales/measures one or more times during the course of, or following, treatment to further assess changes in frequency and intensity of symptoms resulting from treatment.

The present disclosure also provides means for presentation of indices of the psychosis diagnostic index, psychosis risk index, psychosis severity index, psychosis disability index, psychosis cost-care burden index, psychosis symptom(s) frequency index, and/or psychosis symptom intensity index in a longitudinal graphed form or diagrammatic form, to provide easy visual appraisal of progress by clinicians. Such information regarding prevention of illness progression by noting and comparing indices relating to illness regression, remission, relapse, stasis of illness state, resistance of illness state to improvement or change in measures, change in functional outcome measures, change in number, frequency and/or type of risk factors, and/or change in symptom frequency and/or intensity, is then readily available for further case management of the subject.

Methods of the disclosure may also comprise determining the MTHFR 677 genotype and the number or type of abnormal biomarkers described herein for a subject that relate to extended duration of illness (DOI) using correlative strength, logistic regression and Mann Whitney U tests methods to predict DOI related risk factors, biomedical markers and sensory processing variables. Results may then be monitored for pre- and post-treatment changes to evaluate level of prevention or reduction of illness duration from a longitudinal perspective.

Exemplary embodiments of the present disclosure provide a method for determining or predicting prognosis of schizophrenia, schizoaffective disorder or psychosis by determining expected/predicted functional outcome measures for SIR, GAF, SOFAS, expected frequency of hospital admissions, CGI and DSP status, based upon a subject's number-of and/or type-of pathological assessment measures compared with those measures predicted as abnormal outcome measure for their MTHFR 677 genotype in each functional outcome prediction model and expressing this as a ratio or as a functional outcome risk index, value or measure, based upon an algorithm derived from predictive model for each particular functional outcome measures.

Exemplary embodiments of the present disclosure provide a method for determining or predicting cost index and/or care index for a subject with schizophrenia, schizoaffective disorder or psychosis, based on, for example, [determined and/or predicted frequency of hospital admissions]+determined and/or predicted DSP requirement (weighted as 5), presented as an absolute index and/or an index relative to normal controls of the same MTHFR 677 genotype.

Exemplary embodiments of the present disclosure provide a method for determining expected duration of illness (DOI) in years (in the absence of intervention) presented as a duration of illness (DOI) index derived by predictive algorithm or by comparison of type and/or numbers of biomarkers derived from a subject with markers and/or numbers of markers as predicted by the commensurate MTHFR 677 genotype, presented as a risk index for long DOI and/or expected DOI length in years.

Exemplary embodiments of the present disclosure provide a method for determining sensory processing measures and presenting an absolute or relative index measure of sensory processing efficacy and/or deficit, derived from direct comparison with expected/predicted number and/or type of abnormal sensory measures from the commensurate MTHFR 677 genotype and/or determined by a risk-predicting algorithm based upon prediction variables for the commensurate genotype sensory processing model.

Exemplary embodiments of the present disclosure provide a method for predicting number and/or type of sensory processing deficit(s) and/or predicting predominant methylation phenotype and/or typical phenotypic markers based solely upon the prediction profile of abnormal levels, values or ratios presented in each MTHFR 677 genotype model. It is envisaged that this form of genotype based prediction will have particular relevance in country or isolated areas where access to laboratory testing and/or neurophysiological sensory processing assessments may be limited and a shorthand method is required to guide management and pharmacological and other treatment options.

Embodiments of the present disclosure contemplate the employment of one or more additional biomarkers to those described elsewhere herein to aid in diagnosis and risk predictions. Such additional biomarkers may, for example, be used to validate or extend diagnoses made in accordance with the present disclosure. Such additional markers may be markers of, for example, inflammation, tissue damage, oxidative stress, urine excretion function and histamine metabolism. Suitable ‘validation’ markers may include, for example, 1-methyl histamine, histamine, histidine, S-adenosyl-methionine (SAMe), S-adenosyl homocysteine (SAH), ratios between S-adenosyl-methionine and S-adenosyl homocysteine, serum/plasma adenosine, reduced and oxidised glutathione and ratios between reduced and oxidised glutathione, vitamin B2 (riboflavin) and associated molecules such as flavin adenine dinucleotide (FAD), flavin mononucleotide (FMN), F 450, L-threonine, quinone, semiquinone and flavin synthase, urine or plasma L biopterin, tetrahydro-L-Biopterin, (BH4) 7,8-dihydro-L-Biopterin (BH2), BH4:BH2 ratio 5-hydroxy indole acetic acid, platelet monoamine oxidase, red cell N-methyl transferase, catechol-O-methyltransferase polymorphisms, methyl tetrahydrofolate reductase polymorphisms (C667T and A1298C forms), thyroid stimulating hormone, serine, glycine, thromboxane, urine homovallinate, vanilmandelic acid, serum creatinine, immunoglobulins A, E, G, & I, IgG and IgE food allergy screens, IGE allergy correlates, all inflammatory cytokine levels, TNF alpha and interferon kappa B, C reactive protein, serum iron (ferritin, transferrin, transferrin saturation), serum, plasma or urinary lead, antigliadin antibodies, red cell/serum magnesium, serum calcium, free calcium concentration, blood sugar and plasma insulin, N-acetylaspartate, D glucaric acid, phosphocreatinine, glutamate dehydrogenase, N methionine adenosyl transferase, plasma retinol, B-retinylacetate, B-retinoic acid, tyrosine hydroxylase, thyroxine T3, T4 and reverse T3 components, serum creatinine, prostoglandin E1, catalase (CAT), reduced glutathione (GSH), oxidised glutathione (GSSG), antioxidant ratio (GSH/GSSG), vitamin C, albumin, nicatimamide adenine dinucleotide (NADPH oxidase) deficit, glutamate-cysteine ligase (GCL and GCLC), glycerol dehyde-3-phosphate (GAPDH), haeme oxygenase (HO-1), 3-nitrotyrosine (3NT), 8 oxo-deoxyguanosine (8-oxo-dG), 3 chlorotyrosine (3-CT), aconitase activity, H2DCFDA (DCF), advanced glycation end product (CML) or lipid peroxidase marker (HNE). histamine, homocysteine, 5,10 methylene tetrahydrofolate:5 methyl tetrahydrofolate ratio (a measure of MTHFR activity), DOPAC/HVA (homovanilic acid) ratio ((a measure of COMT activity), SAM/SAH ratio, serine:glycine ratio, acetyl choline/choline ratio, phostpatidyl choline, 5HT/HIAA ratio or glutamate/DA, NMDA, red blood cell catechol-0-methyl transferase activity (Kₘ and/or Vₘₐₓ, and/or kcat and/or kcat/Km), FSH, T4 and T3 thyroid hormones, erythrocyte and serum glutathione reductase activity assay (Kₘ and/or Vₘₐₓ, and/or kcat and/or kcat/Km) (a surrogate measure FAD), S-adenosyl-L-Methionine dependent Uroporphyrinogen III Methyltransferase (Kₘ and/or Vₘₐₓ, and/or kcat and/or kcat/Km) (SUMT), Uroporphyrinogen II:siroheme ratio. Fecal count and culture and/or hydrogen and/or methaneand/or carbon dioxide and/or hydrogen sulphide bowel or breath testing. Hydrogen sulphide:methane ratio in breath and feces, fecal short chain fatty acids and/or lactat, serum Fe studies, ferritin, serum iron, siroheme, folacin, calcitonin, L kynurenine:BH4 ratio, kynurenine:quinolinic acid ratio. Urinary excretion of sulphite, thiosulphate, taurine and S-sulphocysteine, protoporphyrinogen IX oxidase gene (EC 1.3.3.4, PPDX).

Messenger RNA gene expression analysis for all genes related to all microbiota and organisms and enzymes mentioned in this application including gene analysis and gene expression analysis of FAD, FMN, B6 and SAMe dependent enzymes, microbiome population diversity analysis of organisms from cecal and/or fecal and/or buccal/oral cavity and/or vaginal bacterial contents for microbial diversity and/or by real time quantitative PCR analysis, northern blotting, microarray analysis, sequencing or next generation sequencing single or multiplex profiling systems and/or fluorescent in situ hybridization (FISH) and/or DNA extraction and/or DNA sequencing, and/or flow cytometry and/or clonal sequencing and/or digestive enzyme analysis and/or RNA-sequencing of bacterial contents and/or 16S DNA pyrosequencing and/or 16S clonal sequencing and/or 16SrRNA gene amplification and sequencing, gene SNP analysis and/or microbiome population diversity analysis, and/or whole-metagenome shotgun sequencing, metatransciptomics, meta-proteomics, sequencing of reverse transcribed RNA transcripts and/or metametabolomics, dynamic allele-specific hybridization, the use of molecular beacons or oligonucleotide single nucleotide polymorphism (SNP) arrays or microarrays, temperature gradient gel electrophoresis, high performance liquid chromatography, high resolution melting of an amplicon, DNA mismatch-binding proteins, SNPlex or surveyor nuclease assay.

Further additional markers that may be measured or assessed in conjunction with the markers hereinbefore disclosed and in accordance with embodiments disclosed herein include, but are not limited to: urinary porphyrins including total urinary haeme, urinary precoproporphyrin (COPRO), keto-isococoporphyrin, urinary uroporphyrin (URO), urinary precoproporphyrin (PRECOPRO), PRECOPRO:URO ratio, uroporphyrin decarboxylase (UROD), cocoporphyrinogen oxidase (CPDX), hepta and hexacarboxyporphyrins, 5-aminolevulinic acid (gamma ALA), urinary coproporphyrinogen and faecal isococproporphyrin); serum/plasma 1 methyl histamine; tGSH:GSSG ratio; glutathione peroxidase; superoxide dismutase; glutathione S transferase P1 (GST P1); glutathion-S-transferase M1 (GST M1); urinary alphahydroxybutyrate; urinary DHPG:MHPG ratio; urinary pyroglutamate; urinary sulphate; urinary 8-hydroxy-2-deoxyguanosine; red cell folic acid; red cell methyl malonic acid; urinary forminoglutamate; serum/plasma adenosine; red cell pyridoxine activation test; red cell transketolase; red cell pyridoxal phosphate activation test; plasma cysteine; total glutathione (reduced) glutathione (GSSG); urinary or plasma tetrahydrobiopterin BH4; red cell pyridoxine activation test; red cell transketolase; urinary xanthurenate; urinary kynurenate; 25 hydroxy cholecalciferol; vitamin D receptor polymorphisms; urinary DOPAC:HVA ratio; vitamins CoQ10, E, A, or D; urinary adipate; urinary suberate; urinary ethylmalonate; APOE polymorphisms; urinary methylmalonate; serum/plasma methionine; serum/plasma S adenosyl methionine; red cell magnesium; serum magnesium; serum Fe 10; ferritin; transferrin; serum cortisol; DHEAS; urine imidazole acetic acid, whole blood histamine, substance P; urinary alpha-ketoisovalerate; urinary alpha-ketoisocaproate; urinary alpha-keto-b-methylvalerate; urinary beta-hydroxyisovalerate; urinary HIAA (5-hydroxyindoleacetic acid); urinary DOPAC (3-methoxytyramine); histidine methyl transferase, urinary HVA (homovallinate); urinary DHPG (dihydroxyphenylglycol); urinary MHPG (urinary 3-methoxy-4-hydroxyphenylglycol); urinary DOMA:VMA; red cell catechol-o-methyl transferase (COMT) including polymorphisms; MRNA for 7 nicotinic acetylcholine receptor, choline creatinine ratio, phosphocreatinine, alpha C-methyl-L-tryptophan trapping, N acetyl aspartate, eosinophil protein X and eosinophil calprolectin, plasma S adenosyl homocysteine; S adenosyl homocysteine hydrolase; platelet catecholamines; urinary hydroxymethylglutarate; blood lymphocyte 7 nicotinic acetylcholine receptor, IGG food allergy screen; imidazole N-methyl transferase, B2 microglobulin; antigliaden autoantibodies (such as tissue transglutamase IGG, tissue transglutaminase IGA, Methionine adenyl transferase, endomysial antibody); urinary p-hydroxyphenylacetate; CD8 and SD4 T cell levels; inflammatory cytokine levels; urine methyl histamine, urine histamine, C reactive protein; erythrocyte or serum N methyl transferase, nerve growth factor; arginine N methyltransferase; urinary VMA (vanilmandelic acid); vesicular monoamine trasnporter (VMAT2); neuronal nitric oxide synthetase; alpha-C methyl-L-tryptophan; acetyl cholinesterase, choline acetyltransferase; vesicular acetylcholine transporter; and tyrosine hydroxylase; red blood cell choline, alpha 7Acetylcholine receptor activity, alpha 4 acetylcholine receptor activity, acetylcholine esterase, glutamic acid decarboxylase, taurine, adenosine, kainate, glycine, spermine, spermidine, glutamate, substance P, aspartate, biotin-quinolones, quinolinate, quinolinic acid, picolinate, kynurenic acid, free androgen index, urinary phydroxyphenylacetate, serum histidine, urinary DOMA (3,4-dihydroxymandelic acid); plasma nitrous oxide; Cu:Zn ratio (N 0.8-1.2); free copper (Cu); urine histamine; plasma chromium; whole blood serum and urine lead (Pb), mercury (Hg) and cadmium (Cd); hair mineral analysis for cadmium, mercury, arsenic, lead, copper, chromium, lithium, sodium, potassium, bismuth and chloride; urine whole blood, red cell and/or serum assays of vitamin A, plasma methyl-malonic acid; plasma, blood and/or urine assay of pyridoxine-5-phosphate (P5P), pyridoxil kinase, niacin, niacinamide, red cell transketolase; thyroid stimulating hormone; thyroid peroxidase antibodies; free T3 and T4; reverse T3; serum cortisol; urinary iodine, urinary folate as urinary fromino-glutamic acid (FIGLU); urinary N-methyl-Nicotinamide.methylmalonic acid; erythrocyte glutamic-pyruvic transaminase (EPGPT); glutamic-oxaloacetic transaminase (EGOT); serum levels of electrolytes, Ca′, Mg′ and BSL; ferritin; biopterin; C-reactive protein (CRP); serum and/or red blood cell assay of manganese; secretory IGA; serum IGA, IGG, IGM and IGE; IGG and IGE for gluten and casein sensitivity; red cell fatty acids; arachadonic acid (AA):EPA ratio; lipid peroxidises; H₂O₂; t-butylhydroperoxide; cumene hydroperoxide; 2-thiobarbituric acid reactive substances (TBARS); apometallathionein; glutamic decarboxylase; oxidative stress biomarkers including 8 hydroxydeoxyguanosine (8-OhdG), malondialdehyde (MDA) and isoprostane; glutathuione peroxidase (GSH-Px); superoxide dismutase (SOD); urine lipid peroxides; hydroxy catechol markers; glutathione transferase; S adenosylhomocysteine hydrolase; spinal motor neuron survival gene (SMN); red cell and/or serum methionine adenosyltransferase; S-adenosyl-L-methionine synthetase; methionine breath test; adenosine deaminase; urinary indicans; valerate isobutyrate; urine analysis of lactulose, mannitol and lactulose:manitol ratio after lactulose mannitol challenge; serum cholesterol; triglycerides; uric acid; serum iron; ferritin, transferrin and transferrin saturation; aspartate amino transferase (ALT), alanine amino transferase (AST); lactic dehydrogenase (LDH); low density lipoprotein (LDL); tissue transglutaminase IgG; tissue transaminase IgA; endomysial antibody; calprotectin and eosinophil protein X; interleukin IB; serum testosterone; free androgen index; DHEAS (dehydroepiandosterone); antigliadin IgA; serum transglutaminase IgA antibody; gliadin IgG antibody; full blood count; haemoglobin; faecal PH; cholesterol; pancreatic elastase; n butyrate; acetate; propionate; faecal total short chain fatty acids; total long chain fatty acids; faecal microbiology, mycology and parasitology; glycine:glucuronide ratio; sulphate:glucuronide ratio; D glucaric acid; glutamate dehydrogenase; urinary amino acids such as histidine, isoleucine, leucine, lysine, methionine, phenylalanine, threonine, tryptophan, valine, cysteine, glutamine, taurine, tyrosine, alanine, arginine, aspartic acid, glutamic acid, glycine, proline, serine, aspartate, asparagine, tyrosine, glutamine and glutamate; copper/zinc superdioxide and catalase activity; ESR; IL-1B (interleukin 1B); tumour necrosis factor alpha (TNFalpha) and serum alpha1, alpha2 and gamma fractions; platelet glutamate levels; serum holotranscobalamin; adenosylcobalamin, NMDA receptor NR2B subunits and other sub unit receptor activity; blood trype; prostoglandin E1; brain derived neurotrophic factor Val/Met polymorphisms; 5HTT-LPR polymorphisms; thiamine; omega 3; omega 6; retinoic acid; bexoratene; UA B30; blood diamine oxidase activity; blood or urine urea; blood or serum thyroxine transthyretin, thromboxane, blood or urine ammonia concentration; urinary amino-n-butyric acid; foramino glutamic acid; urine anserine; urine sarcosine; alpha-ketocaproic acid; beta aminoisobutyric acid.urine glutamic acid; glutaric acid; and glutamine/glutamate ratio; pyroglutamic acid; 3-hydroxypropionic acid. dihidroxyphenylpropionic acid; urine arginine/ornithine ratio; citruline; kynurenic acid; serine; tyrosine; 3 methoxy-4-oh-phenylglycol; taurine; 4-hydroxyphenylpyruvic acid; suberic acid; pyruvic acid; 5 hydroxyphenylpyruvic acid; citric acid; cisaconitic acid; citric acid; aspartic acid; lactic acid; adipic acid; phenyl acetic acid; 5-hydroxy indolacetic acid; dihydroxyphenylpropionic acid; 2-hydroxyphenylacetic acid; cysteine homogentisic acid; benzoic/hippuric acid ratio; lipid peroxidases; carnosine; alpha-amino-n-butyric acid; alpha ketovaleric acid; alphaketomethylvaleric acid; alpha ketovaleric acid; succinic acid; urine beta aminoisobutyric acid; gamma-aminoisobutyric acid; indoleacetic acid; phenylacetic acid; arabinose; malic acid; homogentistic acid; urine methylmalonic acid; urine homocysteine; urine 1-methyl histidine; 3-methyl histidine. urine succinyl purine; inosine; adenosylcobalamine coenzyme; proline; phosphoserine; ethanolamine; urine phosphoserine; urine cystathione; histidine decarboxylase (HDC), histamine-N-Methyl transferase (HNMT), monoamine oxidase A, phosphoethanolamine; orotic acid; urine n methylglycine; urine opiate peptides; IgG and IgE anti casein and gluten antibodies; plasma serine; plasma glycine; serine hydroxymethyltransferase; C¹⁴ or C¹¹ labelled CO₂ following C¹⁴- or C¹¹-methionine administration; histamine N methyl transferase (HMT); serum/plasma glycine; methylcytosine binding protein (MeCP2); histone (H4) deacetylase; acetylated histone (H4); plasma pyridoxyl phosphate; glutathione-S-transferase; cystathione beta synthetase (CBS, CbetaS); cysteine beta synthetase (CBS), S adenosyl homocysteine hydrolase (SAHH), serine hydroxymethytransferase (SHMT), sulphite oxidase, plasma alkalinepyridoxine phosphate phosphatise; mitogen phytohemagglutin (PHA); serum histamine (2-(4-Imidazolyl)-ethylamine); red blood cell histamine; erythrocyte histamine-N-methyltransferase; glycine-N-methyltransferase; retinol binding globulin; glutathione-S-transferase; choline acetyl transferase:to acetylcholine esterase ratio, betaine homocysteine methyl transferase (BHMT), 5 methylhydrpofolate-homocysteine S methyltransferase, methionine synthetase (MS), methionine synthetase reductase, tryptophan hydroxylase, tyrosine hydroxylase, urine dimethyltryptamine (DMT); fasting blood alanine; blood lactate:pyruvate ratio; blood acetyl-carnitine:free carnitine ratio; beta casomorphin-7; casomorphin; influenza titre; glutamic acid decarboxylase 65 & 67 KDA; indoleamine 2,3,-dioxygenase (IDO), tryptophan 2,3-dioxygenase (TDO), reelin proteins; plasma rennin; serine hydroxymethyltransferase; glutathione synthetase; heart rate; blood pressure; continuous task performance; saliva cortisol; catecholamines (noradrenalin and adrenalin and metabolites); corticotrophin releasing factor; coeliac screen, urine propionate, acetate, faecal PH; cholesterol; pancreatic elastase; n-butyrate; acetate; propionate; faecal total short chain fatty acids; total long chain fatty acids; faecal microbiology, mycology and parasitology, hippurate. benzoate, faecal micro-organsim aerobe and anaerobe DNA and mRNA analysis, Glutathione synthetase, glutathione peroxidase, superoxide dismutase (SOD), glutathione-S-transferase P1, glutathione-S-tranferase M1, thiobarbituric reactive sub stances (TBARS), nitric oxide, glutathione peroxidise (GP), copper/zinc superoxide dismutase (SOD), copper/zinc superoxide dismutase, sulphate to sulphide ratio, lipid peroxidases, urine or other bodily fluid ammonia level/concentration, blood ammonia and expired or other ammonia levels, sulphite oxidase, lymphocyte DNA methylation, 8 hydroxy-deguanosine, hydroxyl-2-dehydroxyguanosine, C reactive protein, orotate, tricarb valerate, Kynurenate:quinolinate ratio, glutamate:semiquinolone ratio, dimethyltryptamine L tryptophan, glutamate/dopamine ratio, noradrenaline:substance P ratio, plasma or urinary formate or formic acid, kynurenate/kynurenic acid ratio, lactoferrin, urinary alphahydroxybutyrate, urinary sulphite to sulphate ratio, red blood cell Catechol-o-methyltransferase activity, histamine-n-methyltransferase activity. 3 hydroxy kynurenine, xanthurenic acid, cystathione, neopterin, arginine:citrulline ratio, plasma oxytocin, thioredoxin (TRX), alanine amino transferase (ALT), gamma-aminobutyric acid (GABA), parvalbumin immunoreactivity, flavin adenine dinucleotide (FAD), flavin adenine mononucleotide (FMN), reduced glutathione:oxidised glutathione ratio (GSH/GSSH); quinone, semiquinone and flavin synthase; platelet monoamine oxidase activity (MAO) (Kₘ and/or Vₘₐₓ, and/or kcat and/or kcat/Km), methyl tetrahydrofolate reductase polymorphisms (C667T and A1298C forms), red cell pyridoxine activation test, urinary indicans, interleukin 1B, cholesterol, retinoic acid, glutathione S transferase, glutathione synthase, glutathione peroxidase, catecholamines (noradrenalin, dopamine and adrenalin) and metabolites thereof.

Also contemplated in accordance with the present disclosure is the analysis of gene expression of one or more additional genes, enzymes or other encoded proteins, which can inform and assist in differentiating methylation flux and different mental illness forms. For example, such genes/enzymes may include betaine hydroxymethyl transferase (BHMT), serine hydroxymethyl transferase (SHMT), S andenosyl homocysteine hydrolase (SAHH), methionine adenosyl transferase (MAT) and methionine synthase (MS).

Additional exemplary genes, enzymes and other proteins include: CysG, NAD(P):quinone oxidoreductase (EC 1.6.5.2, NQO1), protoporphyrinogen IX oxidase (EC 1.3.3.4, PPDX), electron transferring flavoprotein (ETF), electron-transferring flavoprotein ubiquinone oxidoreductase (EC 1.5.5.1, ETFDH), glutaryl-CoA oxidase (EC 1.3.3.-, C7orf10), N-5,10-methylenetetrahydrofolate reductase (EC 1.5.1.20, MTHFR), short-, medium-, long-chain acyl-CoA dehydrogenases (EC 1.3.99.2, ACADS; EC 1.3.99.3, ACADM; EC 1.3.99.13, ACADL) and complex I (EC 1.6.5.3, NDUFV1). (OMIM 608801) can be caused by a deficiency of glutaryl-CoA dehydrogenase (type I), electron transferring flavoprotein (type IIA and IIB), electron-transferring flavoprotein-ubiquinone oxidoreductase (type IIC). (OMIM 256000) complex I (containing the FMN-dependent NDUFV1), complex II (FAD-dependent subunit A FOXRED1, (DUOX1-2, FMO1-5 and MAOA+B), at the expense of NADPH, methionine synthase (EC 2.1.1.13, MS), methionine synthase reductase (EC 1.16.1.8, MSR), flavoprotein ubiquinone oxidoreductase (EC 1.5.5.1), flavin-containing monooxygenase isoform 3 (EC 1.14.13.8, FMO3), ACOX1-3, CRY1-2, HAO1-2, MICAL1-3, NOS1-3 and SLC52A1-3), sarcosine dehydrogenase (EC 1.5.99.1, SARDH), N-5,10-methylene-THF reductase (EC 1.5.1.20, MTHFR) riboflavin kinase (EC 2.7.1.26), FAD-adenylyl transferase (EC 2.7.7.2). dimethylglycine dehydrogenase (EC 1.5.99.2, DMGDH) glycine cleavage (EC 2.1.2.10) and serine hydroxymethyltransferase (EC 2.1.2.1), MSR deficiency (OMIM 602568), FAD-dependent protoporphyrinogen IX oxidase (EC 1.3.3.4, PPDX), yridoxine 5′-phosphate oxidase (EC 1.4.3.5, PNPO), allelic variants PNPO (OMIM 603287), phosphopantothenoylcysteine decarboxylase (EC 4.1.1.36; PPCD), COQ6 (EC 1.14.99.-), 3-hydroxy-3-methylglutaryl CoA (HMG-CoA) reductase (EC 1.1.1.34), DUOX2 6 (OMIM 606759). FMN-dependent dehalogenase (IYD 4, OMIM 612025), GLUT1DS 1 (OMIN 606777), FAD exporter FLX1 gene, riboflavin RET1 and RFT2 transporter genes. ribD, ribG and mreA—riboflavin kinase gene, ypaA (ribU), ribM, MCH5, impX gnes Fusobacterium nucleatum, RFT1 and RFT2 genes, ribC and ribR genes, Mali (2-647) Cys310ALA and Cys311 ALA. IDO 1-Indoleamine-pyrrole 2,3-dioxygenase, EC 1.13.11.52 (GC08P039891), also known as indole 2,3-dioxygenase, indoleamine 2,3-dioxygenase 1, indoleamine-pyrrole 2,3 dioxygenase. Aliases CD107B, EC 1.13.11.52, IDO, INDO, INDO (MIM 147435). IDO1mRNA expression; IDO2—indoleamine 2,3-dioxygenase, EC 1.13.11 also known as INDOL1, Indoleamine-pyrrole 2,3-dioxygenase-like protein 1, indoleamine 2,3-dioxygenase 2, indoleamine 2,3-dioxygenase-like 1 protein, indoleamine-pyrrole 2,3 dioxygenase-like 1. IDO2 mRNA expression; Tryptophan 2,3-dioxygenase TDO1 and TDO 2 gene variants, TDO mRNA gene expression. TDO2 (MIM 191070), Tryptophan 2,3-dioxygenase (EC 1.13.11.11), Tryptophan oxygenase (TDO), Tryptophan pyrrolase TO, tryptophan 2,3-dioxygenase TPH2 Tryptophanase TRP; OTTHUMP00000220788; tryptophan pyrrolase (Chromosome: 4; Location: 4q31-q32, Annotation: Chromosome 4, NC_000004.11 (156824847 . . . 156841550), MIM: 191070, ID:), Kynurenine 3-monooxygenase (KMO) (kynurenine 3-hydroxylase) (Chromosome: 1; Location: 1q42-q44. Annotation: Chromosome 1, NC_000001.10 (241695680 . . . 241758944).MIM: 603538, ID: 8564. KMO Single nucleotide polymorphism rs2275163. KMO mRNA expression, Kynurenine amino transferases KAT I & Alpha aminoadipate aminotransferase (KAT II) genes and mRNA expression; Interferon-gamma (IFNG; MIM 147570 Chromosome: 12; Location: 12q14. Annotation: Chromosome 12, NC_000012.11 (68548550 . . . 68553521, complement) MIM: 147570, ID: 3458. Interferon, gamma-inducible protein 30 (IFI30), gamma-interferon-inducible protein IP-30; interferon gamma-inducible protein 30 preproprotein; legumaturain. Chromosome: 19; Location: 19p13.1 (Chromosome 19, NC_000019.9 (18284579 . . . 18288927), MIM: 604664, ID: 10437); Tachykinin peptides neurokinin A, neuropeptide A, neuropeptide K, neuropeptide Y, neurokinin B, Nuclear factor-kappa B (NF-kB) levels and activation. Neurokinin (NK) receptor expression (NK-1, NK-1R, NK 1-RmRNA, NK 1C), NK 2R, NK-3R, neuropeptide Y, NK-B, Substance P receptors NK-1, NK-2R, NK-3R, 14-3-3 regulatory protein (YWHAZ—14-3-3 zeta, also epsilon, gamma, tau, sigma and beta forms), glycohen synthase kinase genes (GSK 3B EC 2.7.11.26, OMIN 605004), Tyrosine kinase neurotrohin receptor TrkB (NTRK2), Calcitonin, Calcitonin gene-related peptide (CGRP), Cystathione beta synthetase (CBS) gene (CBS C 699T, (Y233Y), C1080T, C1956T, CBS (236200), CBS C 699T (Y233Y) C1080T C1958T, C699T, C1956T), SUOX S370S Sulphite oxidase (mutations 606887.0001 to 606887, 1201L, R22211Q, R160Q, G3055, K322R, R309H, Q339R, W393R. SUOX S370S, 606887.0001 to 606887.0004), (TPH) gene variants TPH 191060, IVS7+, 218C-A, tetrahydroisoquinolone levels, Tyrosine hydroxylase (TH) gene variant (276700). Acetylserotonin-o-methyltransferase (ASMT1), Dopamine receptor (DRD4 126452, -1217G), Catechol-O-Methyltransferase genes and variants (COMT-M58525, M65212, V158M, H62H COMT 61=P199P, V158M, V158M (600018)), Monoamine oxidase A genes and variants ((MAO-A) T941G SNP-, MAO-Apromotor R297A, =P199P, MAO-Apromotor 309850.0002 R297A Up/def, T941G SNP, MAO A C to T mutation in position 936, MAO-A mutation A8637, Glutamic acid decarboxylase (GAD65, GAD 65ab, and GAD 67 isoforms, GAD 67, mRNA density, anti GAD antibodies), Serine hydroxymethytransferase gene (SHMT1 C1420T, Cytosolic(17p11.2 isoenzyme, mitochondrial 12q13isoenzyme, Neuroregulin-1\ErbB post synaptic glutamate receptor gene, glutamate receptor gene (mGLuR3), reelin gene (RELN SNPS rs2711870, rs7341475, rs2249372, rs7341474, rs 39329, rs39328, r517746501), Reelin forms (D7S477, D7S1813, D7S5523, D7S495), Reelin isoforms (410-, 330-, 180-Kda proteins, RELNmRNA(s), ApoER2, glutamate cysteine ligase (GCLM) polymorphisms (r52301022, rs3170633), SHANKS 3, gene 22q 13.3, Neuroligin mutation (NLGL b.106, NLGL on Xp22.33, NLGN3 on Xq13, MECP2 on Xq28), serine hydroxy methyl transgerase Gene (SHMT1 C1420T), ACAT1-02, ACAT-02 100678, alpha-7 nicotinic ACH receptors (CHRNA7, NACHRA7, CHRFAM7A on chromosome 15 32.32-32.46 Mb), ncotinicAChReceptor-Alpha Genes (CHRNA1, CHRNA2, CHRNA3, CHRNA4, CHRNA5, CHRNA6, CHRNA7, CHRNA8, CHRNA9, CHRNA10). NAChR Beta GENES (CHRNB1, CHRNB3, CHRNB4, CHRND, CHRNE, CHRNG) muscarinic receptor genes (CHRM2, GRM4. CHRM1, 2 & 5), TPH 191060, IVS7+, 218C-A), tyrosine hydroxylase (TH) gene variant (276700), Acetyl serotonin-o-methyltransferase (ASMT1), serontonin transporter gene (5-HTTLPR), dopamine receptor (DRD4 126452, -1217G polymorphism), Tryptophan Hydroxylase (TH276700); Dopa decarboxylase enzyme (DDC 601 deletion and exon 1, 722-725 deletions), Dopamine transporter gene polymorphisms DAT1 (10-repeat allele, 10R allele, DAT3, VNTR (7-repeat allele), DAT4 (7R allele), DRD5, DRD4 126452-1217G insertion/deletion polymorphism, vitamin D receptor (VDR Taq, MAO-Apromotor 309850.0002 R297A, T941G SNP, Tryptophan Hydroxylase (TPH) gene variants (TPH 191060, IVS7+, 218C-A), 5hydroxytryptophan transporter (5HTT, 5HTTLPR short allele; TPH 1910601V57+218C-A), Acetyl serotonin-o-methyltransferase ASMT1 gene, serotonin transporter HTTLPR V158M, Methyl tetrahydrofolate reductase (MTHFR)polymnorphisms (MTHFR A222V (C6771), MTHFR A1298C, MTHFR 1298A-C[MTHFR A1298C, (E429A), MTHFR-03M, P39P, MTHFR 222Val, A222V (C6771), MTHFR 607093, (thr to met) 607093.0001, missense mutation (arg to gin) 607093.0002], BHMT1 gene (BHMT-01, -02, -04, -08 BHMT HUMAN Q93088; BHMT-01, -02, -04, -08], MTR [1156570, A919G, (A2756G), MTR2756G allele, MTRR 66A-G, MTR 2756A-G, tMTRR 66A-G, MTR 2756A-G, MTR 2756A-G, MTR1156570A919G, (A2756G)5], Methionine synthetase (MS), Methionine synthetase reductase [MTRR H595Y, K350A, R415T, S257T, A919G, (A66G)11, MTRR 66A-G, MTRR 66A-G, MTR 2756A-G], Serine-hydroxymethytransferase (SHMT1-C1420T and 17p11.2 iand 12q13 isoenzyme forms], Histamine-N-Methyl transferase (HNMTD08092, Di6224, HNMT Thr105 variant], AHCY-01, AHCY-02, methionine adenosyl transferase (MAT MIM ID 250850, [MAT1 gene 610550.0001 to 610550.0004, 610550.0005, 610550.0006, R264/R264H dimers of 610550.0007, 610550.0008, RP11-36D19.2, MAT, MATA1, SAMS, SAMS1, MAT 1; MAT-I/III; OTTHUMP00000019971, Location: 10q22, Annotation: Chromosome 10, NC_000010.10 (82031576 . . . 82049434). MIM: 610550, ID: 4143. GC10M082021; MAT1A mRNA expression, GC10M080988 GC10M081253 GC10M082162 GC10M081696], [MAT 2A methionine adenosyltransferase II alpha GC02P085619. MAT 2A mRNA expression, GC05PI62862, MSTP045, MAT-II, MATIIbeta, MGC12237, Nbla02999, SDR23E1, TGR, OTTHUMP00000160916; OTTHUMP00000160917; OTTHUMP00000224275; OTTHUMP00000224294], beta regulatory subunit of methionine adenosyltransferase, MAT2 subunit beta Chromosome: 5; Location: 5q34-q35, Annotation: Chromosome 5, NC_000005.9 (162930231 . . . 162946328). MIM: 605527, ID: 27430. MAT2B mRNA expression]), Reelin mRNA, TXNIP HUMAN Q9H3M7 gene, nitric oxide synthase[(NOS: NOS, E298D, glu298 allele, D298E allele, asp 298 allele, variant (E298D) polymorphism, NOS D298E, gene missense glu298to-asp variant (E298D), glu298 allele homozygosity or heterozygoity; E298D polymorphis, glu298to-asp variant (E298D)], DNA methyl transferases (DNMT 1 and DNMT3Q), BDNF (Met 66, va188 Met (met allele)), Angiotensin Converting enzyme (ACE deletion 16 106180.0001), glutamate receptor mutations (GRIK2 Kainite 2, Common allele variant at 5p 14.1); acetylserotonin-0-methyl transferase gene ASMT1 at 5p 14.1, s 4307059, NSG4 and DISC1.

For biomarkers disclosed herein, measurements may be made of levels, ratios and/or activities, affinity, radioligand binding levels, or other means of biomarker or receptor activation assessment, subunit messenger RNA expression and levels as appropriate. For enzymes, measurements may be of levels, activity, V max and/or Km, kcat, kcat/Km. For genes listed, measurement may be of single nucleotide polymorphisms and isomers, sequence deletions, inclusions, repetitions, isomers, missense mutations, micro DNA or abnormalities of specific interest.

In exemplary embodiments, one or more of the following may also be measured or determined as additional biomarkers: hydrogen sulfide:methane ratio in breath and/or feces; fecal short chain fatty acids; lactate; serum iron; ferritin; siroheme; folacin; L kynurenine:BH4 ratio; kynurenine:quinolinic acid ratio; riboflavin transport saturation kinetics; urinary excretion of sulphites, thiosulphate, taurine and/or S-sulphocysteine.

Biological samples used to determine levels of any biochemical markers contemplated herein may be derived from any suitable body fluid or tissue. For example the sample may comprise blood (such as erythrocytes, leukocytes, whole blood, blood plasma or blood serum), saliva, sputum, urine, breath, condensed breath, amniotic fluid, cerebrospinal fluid or tissue (post-mortem or living, fresh or frozen). In a particular embodiment the sample comprises whole blood, blood serum or urine. In specific embodiments of the present invention the markers in the neurotransmitter domain and the nutrition-biochemistry domain are typically determined from blood or urine samples obtained from an individual to be assessed, more typically from blood samples. The markers in the oxidative stress domain are typically determined from urine samples obtained from an individual to be assessed.

Any suitable techniques may be used to measure or determine biomarker levels in addition to the techniques and methods described and disclosed elsewhere herein. Such additional techniques and methods may include, for example, biophotonic and hyperspectral imaging analysis, iphone well tests, gene chip hybridization, infra red spectroscopy, multilocus enzyme electrophoresis, mass spectroscopy and mass spectroscopy footprinting, 5S rRNA and 16S rRNA sequence analysis, X-ray crystallography, homologous structure modelling, genome wide transposon mutagenesis and insertion site sequencing, multiplex pyrosequencing of PCR products, shotgun sequencing of fecal sample DNA.

Diagnoses of a number of additional conditions may be effected using the methods of the present disclosure such as, for example, developmental abnormalities, PTSD, iritible bowel syndrome, colon cancer, autism, attention deficit disorder (hyper active and inattentive forms), bulimia, binge eating disorder, obesity, motor neuron disorder, amyotrophiccc lateral sclerosis, chronic fatigue syndrome, hypomanic, anxiety, mixed affective state, dyslexia, coordination disorder, catatonia, extra pyramidal side effects, overeating, poor appetite, psychomotor retardation, alexothymia, asthma, delayed sleep phase disorder, and atherosclerosis.

### Treatment

One advantage offered by the diagnostic methods of the present disclosure is the ability to reveal to the clinician objective evidence of a subject's areas of pathology and unmet needs in areas such as abnormal neurotransmitters, vitamins and minerals and oxidative stress status. From this position, the clinician can initiate specifically targeted remedial interventions. For example, an individual with wild type (CC) or heterozygous (CT) genotype could be treated with supplementation of riboflavin, with or without further supplementation with with vitamin D and/or vitamin B6. In embodiments, treatment may also comprise supplementation of vitamin B2 or flavins, for example with a vitamin-comprising composition or B2 producing probiotics (including Bacillis subtilis or Saccharomyces cerevisiae). In further embodiments, treatment may also comprise administration of zinc to normalise methylation cycle, or glycine and/or L-threonine to boost the tricyclic acid cycle. Lecithin (choline) may be administered to boost acetylcholine production. In another embodiment, N-acetyl choline (NAC) can be administered to assist with antioxidant defense. In an embodiment, BHMT activity can be increased by a low protein diet, as low methionine increases its activity.

The inventors have designed suitable treatment regimes for subjects suffering from schizophrenia, schizoaffective disorder and/or psychosis, based upon determination of the subject's MTHFR 677 genotype and the levels of biomarkers in the subject as described herein and measures of sensory processing variables as described herein, in the presence or absence of a treatment regime for treating the schizophrenia, schizoaffective disorder or psychosis. These treatment regimes may comprise adjusting the identity, timing and/or intensity of the treatment regime so as to normalise any and or all levels, measures, values or ratios of one or more of the biomarkers. In an embodiment, the disclosure provides a method of treating one or more symptoms in a subject by selecting subjects that meet the criteria for an adequate or over-methylation state based on their biological profile.

Accordingly, the present disclosure provides methods for determining effective treatment regimes for sufferers of schizophrenia, schizoaffective disorder and/or psychosis, including subtypes of schizophrenic and schizoaffective psychosis, by carrying out diagnostic tests as described herein, optionally over time and determining if there is a change over time concomitant with, or resulting from, the employment of a specific treatment regime. Also provided are methods for monitoring treatment regimes, including monitoring for treatment progress, preventing patient relapse or managing treatment resistance, by carrying out diagnostic tests as described herein over time and determining if there is a change over time concomitant with, or resulting from, the employment of a specific treatment regime. Thus, a subject's clinical progress over time may be assessed in terms of, for example, remission, regression, relapse, and/or rate of symptom frequency and or intensity.

An exemplary embodiment provides a method for evaluating the efficacy of a treatment regime in a subject with a psychotic disorder such s schizophrenia, schizoaffective disorder or psychosis, the method comprising:
(a) treating the subject with a treatment regime for the psychotic disorder for a period sufficient to evaluate the efficacy of the regime;
(b) obtaining one or more biological samples from the subject;
(c) determining the status of the MTHFR C677T polymorphism, and optionally determining measuring or assessing one or biomarkers and/or additional parameters as defined herein in the one or more biological samples;
(d) repeating steps (b) and (c) at least once over a period of time; and
(e) determining whether the levels, values or ratios for the one or more biomarkers and/or additional parameters change over the period of time.

Disease control in the subject may then be improved by adjusting the timing, frequency and/or intensity of marker testing and/or adjusting the identity, timing and/or intensity of a treatment regime to thereby normalise the levels of one or more of the markers.

The term “disease control” as used herein means the status of the schizophrenia, schizoaffective disorder or psychosis, typically in light of treatment or therapy intervention. Thus “disease control” describes the range and severity of symptoms and conditions experienced and suffered by patients as a result of their schizophrenia, schizoaffective disorder or psychosis. Disease control effectively provides a measure at a given point in time of the disease status of an individual, reflecting both current therapeutic treatment regimes used by the individual and the individual's recent experiences and psychological state.

Also provided in an exemplary embodiment is a method for designing a suitable treatment regime for a subject suffering from schizophrenia, schizoaffective disorder and/or psychosis, the method comprising determining the MTHFR 677 variant and the levels of biomarkers in the subject as described herein, in the presence or absence of a treatment regime for treating the schizophrenia, schizoaffective disorder or psychosis and adjusting the identity, timing and/or intensity of the treatment regime so as to normalise the levels of one or more of the markers.

In another aspect, the present disclosure provides a method of preventing progression of a psychotic disorder in a subject, including the steps of assessing the subject for a psychotic disorder or risk of developing a psychotic disorder based upon their biological and sensory-processing; including risk of disability, occupational and social functioning decline, severity, hostility, suicidality; selecting subjects that meet the criteria for developing a psychotic disorder based upon their biological profile; selecting subjects that meet the criteria for treatment and administering to the selected subject a composition appropriate to the status of their biological markers.

In exemplary treatments and treatment regimes, the methods may provide for an initial medical and/or intervention response to confounding conditions that may adversely affect MTHFR 677 genotype and methylation-guided treatment proposed. For example, if cortisol is high, the causes of high cortisol, stress, and/or risk factors may be investigated and treated with risk factor reduction intervention strategies, stress reduction or/and cortisol-reducing management and agents. If thyroxine is high, the cause of hyperthyroidism may be investigated and treated. If thyroxine is low, the cause of hypothyroidism may be investigated and treated. If hearing and/or vision pathology, extra-pyramindal symptoms or mental and/or motor incapacity is detected and/or external or internal ear pathology and/or infection, the cause may be investigated and treated. If thyroxine T4 (or T3) is low, investigating the cause of hypothyroidism and treating with thyroid hormone support since thyroid hormone may be required for conversion of riboflavin into FMN (low thyroid hormone may therefore constitute a cause for insufficient FMN synthesis). Antihistamines may be administered if gastrointestinal problems, autoimmune or other allergy problems or high histamine levels are present.

In exemplary embodiments, subjects may be administered one or more of the following on a daily or twice daily basis: zinc picolinate at, for example, about 50 mg daily or twice daily; pyridoxal-5-phosphate at, for example, about 5 to about 25 mg daily or twice daily; nicotinamide (vitamin B3) at, for example, about 10 mg to about 500 mg daily or twice daily; riboflavin or riboflavin 5-phosphate at, for example, about 5 mg daily or twice daily; thiamin (vitamin B1) at, for example, about 10 mg daily or twice daily; folinic acid at, for example, about 840 mg daily or twice daily; methionine at, for example, about 750 mg daily or twice daily; ascorbic acid at, for example, about 300 mg to about 500 mg daily or twice daily; vitamin D3 at, for example, about 1000IU-2000IU daily or twice daily; ferrous sulphate at, for example, about 325 mg daily or twice daily; sodium butyrate at, for example, about 600 mg daily or twice daily; folic acid at, for example, about 267 mg daily or twice daily; vitamin B12 (cyanocobalamin) at, for example, about 17 mg daily or twice daily; betaine (trimethylglycine or TMG) at, for example, about 400 mg daily or twice daily; and/or methylcobalamin at, for example, about 1000 mg daily or twice daily.

Also contemplated by the present disclosure is the investigation and monitoring of the microbiome of a subject, and administration of suitable probiotic or other microorganisms as appropriate and as needed, with reference to the determined MTHFR 677 genotype of the subject and their associated psychosis phenotype. For example, administration of riboflavin synthesizing bacteria, yeast and plants, sulfur reducing microorganisms, methangens or obligatory riboflavin digesting organisms may be appropriate. Exemplary riboflavin synthesizing bacteria, yeast and plants include Bacillus subtilis, Bacillus flavinia, Escherichia coli, Methanocaldococcus jannaschii, Methanobacterium thermoautotrophicum, Pseudomonas fluorescens, Enterobacter spp., Corynebacterium ammoniagenes, Lactobacillus lactis, Lactobacillus plantarum, Leuconostoc mesenteroides, Propionibacterium freudenreichii, flavinogenic fungis such as Ashbya gossypii, Saccharomyces cerevisiae (Baker's yeast), Ashbya gossypi (fungus), Eremothecium ashbyii, Candidafamata, Pichia guilliermondii, Schizosaccharomyces pombe, and Arabidopsis thaliana Exemplary sulfur reducing microorganisms include Escherichia coli, Desulfovibrio desulfuricans, Desulfovibrio piger, Desulfovibrio intestinalis, Bacteroides fragilis, Helicobacter pylori, Staphylococcus aureus, Lactobacillus spp. including, for example, L. reuteri, L. casei, L. helveticus, L. plantarum, L. rhamnosis, L. brevis, L. buchneri and L. paracasei, Fusobacterium spp., Streptococcus spp., Enterococcus spp., Enetrobacter spp., Klesbiella spp., and Prevotella spp. Exemplary methanogens include Methanobrevibacter smithii, Methanobrevibacter oralis, Methanosarcinales spp. Methanocaldococcus jannaschii, Escherichia coli, and Clostridium spp. Exemplary obligatory riboflavin digesting organisms include Corynebacterium pyogenes, Streptococcus pyogenes, Listeria monocytogenes, Lactobacillus casei, Rickettsia spp., protists, and spirochetes.

Those skilled in the art will appreciate that these dosages are exemplary only and may be adjusted depending on a variety of factors such as the particular agent or composition being administered including the form in which the agent or composition is administered, the age, body weight, general health, sex and dietary requirements of the subject, as well as any drugs or agents used in combination or coincidental with the agent or composition. Those skilled in the art will appreciate that single or multiple administrations may be carried out in accordance with the present disclosure with dose levels and dosing regimes being determined as required depending on the need of the subject and on the condition of the subject to be treated. For example, several divided doses may be administered hourly, twice daily, daily, weekly, monthly or at other suitable time intervals or the dose may be proportionally increased or reduced as indicated by the exigencies of the situation. Based on the teaching herein those skilled in the art will, by routine trial and experimentation, be capable of determining suitable dosage regimes on a case-by-case basis.

Those skilled in the art will also appreciate that the treatments offered to subjects in accordance with the present disclosure will typically depend upon the determined MTHFR 677 genotype and the associated psychosis phenotype. Hereinbelow are exemplary illustrations of suitable treatments for subjects with either of the MTHFR 677 genotypes in different scenarios based on determinations of biomarker levels and/or ratios as described above and on the measurement or assessment of additional parameters as described above.

### Treatments—MTHFR 677 CC Variants

As exemplified herein, in the case of the wild type MTHFR 677 CC variant, elevated HPL/SG is associated with higher number of sensory processing deficits and other under-methylating characteristics within the phenotype of this wild type MTHFR 677 CC variant. Without wishing to be bound by theory, elevated HPL levels in the setting of this variant is related to riboflavin metabolites and HPL itself is related to pre-ageing of sensory processing neural circuits with difficulty sustaining auditory and visual working memory and sensory integration. Both riboflavin and HPL are degraded by exposure to UV light with release of reactive oxygen species that may be responsible for oxidative damage to sensory neural circuits, so is likely that elevation of both products in the body and urine and neural damage may be to some extent avoided by avoiding exposure to UV light. HPL is also significantly elevated in the MTHFR 677 CC variant and its cleaved structure does bear a relationship to riboflavin and its degradation product lumichrome. Riboflavin may lose its ribytyl side chain in the process of oxidation and be converted to lumichrome and/or lumiflavin. This may occur through it complexing with tryptophan in an oxidative reaction that facilitates cleavage of the central isoalloxazine ring structure of riboflavin and oxidation of its 7 and 8 methyl groups to the level of hydroxymethyl flavin, which has a structure resembling that of HPL. Mycobacterium and Methanogens in the gastrointestinal microbiome may also excessively degrade riboflavin (vitamin B2) as part of F420 production produced by an anerobic heme synthesis pathways that utilise D-amino levulinic acid (ALA) as the first tetrapyrrole/heme precursor derived from glycine, acetyl CoA and serine. ALA is then converted by the vitamin B6 utilising enzyme Amino Levulinic Acid Synthase (ALAS) and the zinc dependent enzyme Porphyrobilinogen synthase (PBG synthase), to Uroporphyrinogen III. From Uroporphyrinogen III, an anaerobic route to precorrin and siroheme synthesis branches off from the usual aerobic heme-synthesising route pathway and this may be used by bowel methanogens. The usual pathway for progressive pyrrole/porphyrin degradation towards pyrrole fragmentation (like HPL) forms protoporphyrin IX, which is oxidised by a FAD dependent enzyme protoporphyrinogen IX oxidase (PPDX)), so this pathway may become progressively unable to proceed to degrade pyrrole porphyryn through the usual heme oxidation and biliverdin reductase route. The alternate anaerobic pathway leads to corrin, redox cofactor F420 and siroheme biosynthsis which traps iron. Corrin requires SAMe for its synthsis and also utilises SAMe to form vitamin B12, which is particularly elevated in the MTHFR 677 TT setting, where SAMe is readily available. Meanwhile, the versatile redox cofactor F420 can plays a side-role in degredation of a flavin precursor deazoflavin, which may explain low riboflavin levels in this setting, followed by low riboflavin-related activation of vitamin B6 and vitamin D, providing a profile similar to that of the undermethylation profile in the wild type MTHFR 677 CC variant. Since both SAMe and FAD are deficient in the MTHFR 677 CC setting, tryptophan can complex with riboflavin and oxidatively-cleave its isoalloxazine ring system riboflavin so that it may be degraded by F420 to HPL, however when SAMe and FAD are in surplus, as in the MTHFR 677 TT setting, corrin-B12 synthesis by gastrointestinal methanogens in the anaerobic Uroporphyrinogen III pathway of heme synthesis, may combine with the usual aerobic body mechanism for heme oxidation which eventually via biliverdin reductase to an oxidised, degraded methylated HPL pyrrole fragment.

This proposed differing dynamic of HPL by methanogens between CC and TT settings, may explain the divergent findings relating to HPL in this project, where HPL correlates most highly with the MTHFR 677 TT variant in a setting where vitamin levels and sensory function are preserved, however appears related to adverse sensory function in the MTHFR 677 CC variant, where toxic oxidative tryptophan complexes can complex with it in an oxidative fegredation reaction which may be assisted by F420 to form HPL. In such a setting, activated vitamin levels are low, such that the combination of excess oxidative degradation, anaerobic methanogen activity and relative vitamin B6 and D deficits leads to neuro-oxidative-nutritional starvation of sensory processing pathways with consequent sensory processing deficits. In view of the above potential for HPL formation to relate to excessive degradation by methanogens in the microbiome, exemplary treatment options may include investigation of a subject's fecal microbiome early in the course of psychotic or other abnormal mental state and/or prior to psychosis onset.

In view of the imputed role of methanogens such as Methanobrevibacter smithii, Methanobrevibacter oralis, methanosarcinales, Methanocaldococcus jannaschii, e coli and some clostridia species, in anaerobic degradation of riboflavin as a mechanism to explain low vitamin B2 levels in MTHFR 677 CC variant phenotypes and conserved vitamin B2 levels in MTHFR 677 TT variant, sulfur reducing bacteria such as Bacteroides fragilis, desulfovibrio spp; fusobacterium spp, enterococci and lactobacilli sp, may have a carefully-monitored role in modifiying the MTHFR 677 CC microbiome and inhibiting anaerobic methanogen activity. Where it has been pre-determined that methanogens occur in excess, dietary modification to encourage growth of sulfur reducing bacteria through dietary manipulation with a high fat, high sulphate diet via addition of sulfur-containing foods such as milk, eggs and cheese, dried food, brassica vegetables and rhizomes, may stabilise vitamin B2 levels and restore levels of vitamin activation, as well as provide sources of riboflavin itself.

Treatment options in accordance with the present disclosure include the administration of riboflavin or prodrugs or derivatives thereof in those MTHFR 677 CC subjects in which the diagnostic and prognostic tests, measurements and assessments described herein indicate that riboflavin levels are deficient or riboflavin degradation exceeds synthesis. Riboflavin may be administered singly or in combination (simultaneously or sequentially) with, for example, active forms of vitamin D and/or vitamin B6. Recommended riboflavin intake (in the UK), ranges from 0.4 mg/din infancy to 1.3 mg/day in adults with an increment of 0.3 mg in pregnancy and 0.5 mg during lactation to cover increased tissue synthesis for fetal and maternal development and riboflavin secretion in milk. The safe supplement maintenance dose is 15-25 mg every two weeks, however larger doses of up to 200 mg to 300 mg per day have been reported effective. Alternatively, or in addition, administration of nicotinamide (niacinamide or tetranicotinate) in low dose, may be warranted, noting that in the undermethylating phenotype there is insufficient SAMe to metabolise any tryptophan that is degraded to niacin by bacterial mitochondria.

Commercial riboflavin is typically currently produced by microbial synthesis using special selected strains of bacteria, yeasts and fungi which comprise Saccharomyces cerevisae in bakers yeast, non-pathogenic E. Coli, Bacillus subtilis, Ashbya gossypii, Candida famata, Bifidobacteria species, Schizosaccharomyces pombe, Eremothecium ashbii, Pichia guilliermondi, Candida flaveri, Corynebacterium ammoniagenes, bacillus flavinia and/or Lactobacilus species such as Lactobacilus lactis and L. plantarum, lactobacillus fermentum. Other substances which enhance riboflavin synthesis are glycine and ribityl.

Riboflavin may also be provided in one or more naturally occurring or manufactured foods or foodstuffs, such as bakers yeast, brewer's yeast, vegetable oil, fungi, almonds, maternal and other milk, cereals, fatty fish, and leafy vegetables (such as spinach, broccoli, brussel sprouts) and/or as riboflavin-5-phosphate and/or riboflavin butyrate. Vitamin B6, zinc, vitamin D and folate or methylating agents such as betaine, choline and methionine can be added as adjuncts.

If AD/NA is low and Vitamin B12/Vitamin D ratio is high, this would indicate predominance of under-methylation. Treatment with methylating and other suitable agents in the setting of MTHFR 677 CC may take form of, for example: S adenosyl methionine (SAMe), at for example about 100-200 mg per day; methylcobalamin at for example about 1000-6000 mcg per day; betaine, choline and/or methionine; methionine synthesising bacteria as probiotics, such as S. faecalis, L mesenteroides, non-pathogenic E. coli and Saccharomyces cerevisiae; serine, vitamin B6 and/or N acetyl choline; riboflavin stabilizers (retard degradation) such as dimyristoyl-phosphatidylcholine (DMPC) to maximise stability and protect against degradation, disodium ethylenediamine (EDTA), thiourea, methylparaben, DL-methionine, sodium thiosulfate, ribonucleic acid and reduced glutathione.

If AD/NA is low or NA/AD is high and/or NA MHMA is high, treatment with noradrenergic receptor antagonists such as clonidine or guanefecine may be considered. If NA/AD is high, beta blocking agents such as propanalol or Timolol may be considered. If NA/DA is low or DA/NA is high, dopamine blocking antipsychotics may be considered. If AD/MHMA is high, consideration of alpha 1 receptor antagonists such as praozin. If 5HIAA is high, in the MTHFR 677 CC setting, consideration should be given to withholding serotonin reuptake inhibitor medication. If AD/NA is high and/or NA/DA is high and/or DA/NA is low and/or % free copper to zinc ratio is high, DA antagonizing antipsychotic agents should be withheld and the patient monitored for low dopamine “parkinsonian” or extrapyramidal side-effects. In addition, if AD/NA is high, there may have been a compensative switch to an over-methylation state, in which case, such treatment as described above should be reduced or discontinued and supplementation switched to low dose niacin or niacinamide (also known as nicotinamide), (50 mg daily dose), considered, since switch from under- to over-methylation will shift niacin from being under-synthesised and under-utilised to over-metabolised by switch from low SAMe to high SAMe, respectively.

Serine metabolism is expected to be halted and serine trapped by insufficient FMN-activated B6 in a MTHFR CC setting where B6 is required for its metabolism to glycine. Therefore what serine is synthesised, may itself be trapped along with its product substance L tryptophan, which is similarly trapped and unable to be metabolized in the absence of FAD and B6 for tryptophan pyrrolase activity. In this setting, lesser dietary serine and L tryptophan may be indicated, with due regard to the fact that sufficient serine is required for phosphatidyl serine cell wall support and for purine synthesis and DNA maintenance. Since serine is a precursor of tryptophan and both serine and tryptophan are trapped in the MTHFR 677 CC state, prescribing of SSRI's may be necessary in order to conserve serotonin in a setting where vitamin B6 and vitamin B2 supplementation is not occurring. The requirement for additional methyl groups in this MTHFR CC setting cannot be ignored and methionine supplementation and/or a meat diet may assist to boost methylation. Protein dietary inclusion may also slow serotonin elevation in the brain by providing competing amino acids for blood-brain barrier protection against elevated levels of trapped L tryptophan.

The activity of the compensative BHMT pathway may be monitored by assay of methylation status related biomarkers as described, to ensure that a switch to over-methylation has not occurred. If this is not the case, methylation can be boosted and homocysteine levels reduced from about 27% reduction to about 83% reduction by enhancing the BHMT pathway precursor loading with betaine (trimethyl glycine (TMG)) and/or choline loading. These amino acids are plentiful in a high protein meat-eating diet. N acetyl cysteine and/or serine may boost glutathione formation and assist with antioxidant defense. Antipsychotic and anti-depressive medication may be useful for carriers of the riboflavin-poor MTHFR 677 CC variant.

Establishing the biomedical phenotype with respect to catecholamines is helpful early in illness course, as when DA/NA ratio is high, and/or the % free copper/zinc ratio is low, administering dopamine blockading medication is likely to be successful in reducing symptoms. Conversely, withholding dopamine blockading medications is a better course when the DA/NA ratio is low and/or NA/DA ratios are high and/or the AD/NA ratio is high and/or the NA/AD low and/or the % free copper to zinc ratio is high, in which case administering beta blocker medications such as propanalol may provide better efficacy for symptom reduction as first line or adjunctive treatment. The alpha 2A blocking medication guanfacine is a further option for controlling anxiety through adrenergic receptor blockade and improving spatial working memory and reaction time. Probenecid decreases the renal clearance of riboflavin so may have a therapeutic role to conserve riboflavin for carriers of the MTHFR 677 CC variant.

### Treatments—MTHFR 677 TT Variants

A subject with the MTHFR 677 TT genotype may have low or not-elevated NA/MHMA and/or AD/MHMA ratios, or the subject may have high AD/NA, high or low DA and/or high homocysteine. The subject may also have high or normal levels of vitamin B6 and vitamin D. Vitamin B2 levels and ratios of Peak 2 (riboflavin) to Peak 1 (riboflavin metabolite) amplitudes and areas under peaks on HPLC assessment of riboflavin and metabolites may be elevated or normal relative to controls. In addition, riboflavin and/or FMN and/or FAD may be high or normal relative to controls. Zinc may be low and % free copper may be high, therefore % free copper to zinc ratio may be high. The subject may have high SAMe/SAH ratio, over-metabolised, over-utilised low niacinamide (niacin or nicotinamide), low histamine, high cortisol, normal or low serine, high or normal acteylcholine, high or low DA and/or high or normal HPL.

Elevated DA synthesis is encouraged by SHMT activity upstream of the inactive MTHFR 677 TT coded enzyme and SHMT must therefore divert its one carbon substrates towards the tetrahydrobiopterin pathway and indole catecholamine synthesis, with potential for raised dopamine and serotonin. In the homozygous 677 TT-coded enzyme setting, treatment of elevated dopamine levels needs to be clearly understood because although dopamine synthesis is facilitated by ready availability of activated vitamin B6 facilitating serine metabolism's spin off effect on the biopterin cycle, such DA levels can quickly collapse in the face of SAMe's facilitation of catechol-o-methyl transferase enzyme which metabolises catecholamines. If this catecholamine metabolism dynamic takes sway over dopamine synthesis, and/or underlying adrenal, gut and renal systems can no longer keep up with catecholamine synthsis demands, DA levels may fall precipitously with negative symptoms, motor retardation, neuro-vegetative and/or endogenous depressed mood and suicide risk. For this reason. adrenal function and DA levels need careful monitoring, with a cautious approach taken to any dopamine receptor blockade medication. Such caution may also be neccessary in a setting of low zinc or high Cu, where DA metabolism by dopamine beta hydroxylase is copper facilitated. In such a setting of risk of rapid conversion to a low DA state, use of dopamine-blockading medications may provide a false sense of therapeutic security and their overlay effect of dampening dopamine neurotransmission in an already-reduced dopamine milieu, will carry risk of inducing extrapyramidal side effects, neuroleptic-induced deficit syndrome and/or even catatonia. Suicide risk also needs to be carefully considered against background of homozygous MTHFR 677 TT genotype and/or low dopamine biochemistry results. In such a setting, slow release dopamine agonists such as methylamphetamine may provide a preferable boost to dopamine transmission and if adrenal exhaustion is indicated by low NA and AD levels there may be a role for beta 2 adrenergic agonists such as salmeterol, formoterol, indacaterol.

Unless NA/DA is high and/or DA/NA is low due to high copper levels, zinc supplementation may be required to overcome the high zinc cofactor utilization brought about by increased compensative BHMT activity and to support methionine synthase as the normal methylation cycle is re-established. If NA/DA is high, beta catechol blockers can be considered as treatment. If NA/DA is low and/or DA/NA is high, vitamin C should be supplemented and/or % free Cu/Zn checked and if appropriate, copper supplemented. DA antagonising antipsychotics may assist in symptom reduction and treatment, however if % free copper to zinc ratio is high and/or if NA/DA is high, and/or DA is low and/or DA/NA is low as may occur in the MTHFR 677 TT setting, dopamine-antagonist antipsychotic agents should be withheld and the subject monitored for low dopamine “parkinsonian” or extrapyramidal side-effects and/or negative deficit syndrome and/or neuroleptic induced deficit syndrome and/or catatonia and/or depression and/or suicidality. There may also be a need for antiparkinsonian medication. Acetyl choline levels may be raised in a setting of high serine to glycine choline turnover and anticholinergics may reduce spasticity effects of parkinsonism, however their reduced gastrointestinal transit time may enhance riboflavin absorption, so contrasting side effects need to be carefully monitored. If AD/NA is high in a setting of paranoia and/or anxiety, Alpha-1 adrenergic antagonists such as praozin may be considered and/or administered with due regard to their hypotensive effects.

Niacin (niacinamide) is metabolized to SAH using up methyl groups supplied by SAMe, and thus lowering methylation processes fueled by SAMe. Therefore higher niacinamide doses are required in adults with the MTHFR 677 TT genotype, such as from about 50 to 3000 mg daily. Niacin may also be administered via, for example about 500 to 2,000 mg of extended-release niacin and/or about 2% to 5% niacin in hand cream daily. Males typically require 16 mg of niacinamide per day, while women and adolescent girls need 14 mg. Pregnant or breast-feeding women typically require higher doses of niacinamide, increased to 18 mg and 17 mg, respectively. Dietary forms of niacin include beans, green vegetables, liver, mushrooms, peanuts, whole wheat, unpolished rice and enriched flours. Although milk and eggs do not contain niacin, they are rich in tryptophan and body can produce about 1 mg of niacin daily by converting 60 mg of dietary tryptophan.

Subjects with over-methylation related to the MTHFR 677 TT variant do not require sources of methionine or extra sources of vitamin B2, therefore in symptomatic states, dietary meat (methionine) and dairy (riboflavin) intake may be reduced and/or limited. Intake of any or all of methionine, folic acid, serine, choline, glycine and vitamin B6 (and if FAD or FMN is high, riboflavin) may also be reduced and/or limited. Novel riboflavin absorption and/or transport competitors/inhibitors such as glucose and glycerol may be considered.

If riboflavin levels or Peak2 amplitude and/or area under peak 2 values are high, this indicates that riboflavin synthesis by gut bacteria and/or yeasts is exceeding their riboflavin degradation. In such a setting microbiome examination may include investigation of level of riboflavin synthesising bacteria and/or yeasts. Exclusion and or finding and treating Candida albicans or other yeast infection with metronidazole or Nystatin may be a priority in the MTHFR 677 TT genotype, since yeast species may over-produce riboflavin. Where riboflavin synthesising species are found to be in excess, probiotic and/or fecal replacement with riboflavin-degrading methanogens such as Methanobrevibacter smithii and/or oral transplant withs Methanobrevibacter oralis may be considered. Riboflavin quenchers include ascorbic acid, sodium azide, β-carotene and lycopene, glutathione, D-mannitol, phenol foods, polyphenols such as catechin, epigallocatechin, and rutin, potassium iodide, purine derivatives such as uric acid, xanthine, hypoxanthine, α-, β-, γ- and δ-tocopherols, vitamin B6, xanthone derivatives 1,4-diazabicyclo[2,2,2]octane and 2,5-dimethylfuran. The fungus Schizophyllum commune degrades flavins and lumichrome, which may be the precursor of HPLC Peak 1 flavin metabolites, so may provide a novel treatment agent for elevated HPL. In this B6 sufficient setting, elevated HPL also responds to zinc supplementation.

In view of low zinc levels due to high zinc utilisation by BHMT, zinc supplementation may have the advantage of lowering free copper levels and encourage copper inhibited enzymes such as CBS and MS, to re-establish their activity.

In the MTHFR 677 TT genotype, L tryptophan is well-metabolised via B6-facilitated tryptophan pyrrolase in the kynurenic pathway and in a setting of plentiful vitamin B6, its metabolism is diverted to synthesise niacin and niacinamide in the transulfuration pathway, Niacin and niacinamide can mop up excess methyl groups by assisting in SAMe conversion to SAH. Depressed mood and motor retardation symptoms in the MTHFR 677 TT state usually occur as a result DA depletion and not related to serotonin deficiency. In settings of inflammation, autoimmunity and/over-methylation, L tryptophan (the precursor substrate for serotonin synthesis) may be preferentially metabolized by tryptophan pyrrolase along the kynurenic pathway to eventually reach niacin synthesis. Subjects may be monitored for inflammation, elevated cortisol and/or low tryptophan levels. Inflammation may be investigated by monitoring cytokine levels, TNF alpha and C reactive protein levels. If CRP exceeds 3 mg/L treatment with anti-inflammatory medication such as IDO inhibitors (e.g. L methyl 2 L tryptophan (IMT) or TDO inhibitors such as allopurinol or anti THF alpha agents such as infliximab) may be considered. If, as a result of inflammation, low serotonin levels are detected on assay, judicious use of serotonin-conserving SSRI medication can assist in maintaining serotonin balance.

Alternatively, tryptophan levels may be boosted by ingestion of foods high in tryptophan such as chocolate, oats, dried dates, milk, yogurt, cottage cheese, red meat, eggs, fish, poultry, sesame, chickpeas, almonds, sunflower seeds, pumpkin seeds, buckwheat, spirulina, and peanuts, which are also capable of chelating excess riboflavin. L tryptophan can also be synthesised from serine by a fermentation process within gastrointestinal bacteria such as B. subtilis, or E. coli.

Probiotic bacteria that readily metabolise riboflavin such as methanogens, Mycobacterium bovis, Mycobacterium fortviitum, Mycobacterium jannaschii and Pseudomonas riboflavin bacteria may also provide an oral treatment method. Riboflavin analogues such as roseflavinol, calmidazolium or trifluoperazine can also influence the riboflavin absorption pathway and reduce flavoproteins.

### Treatments—MTHFR 677 CT Variants

Due to the potential to rapidly cycling between under and over methylation states where mood is labile and anxiety or frustration is high, frequency of biochemical testing may be precluded in the MTHFR 677 CT setting, in which case sodium valproate or other mood stabilizing medication along with stress reduction and/or anxiolytic medication, may be the most effective treatment. If the AD/NA ratio is high, subjects with a mixed methylation phenotype may be administered niacin or nicatinamine. Also, an alpha 1 antagonist such as proazin and/or GABA analogue anticonvulsant such as sodium valproate, pregabain, gabapentin, progabide. Stabilising arousal and mood shifts with, germinated brown rice or probiotic mixture oif GABA producing bacteria such as Streptococcus therophilus, Lactobacilus brevis, L. Buchnieri and L paracasei. In such settings, noting and modifying dietary intake changes between high and low fat and high and low sulfur dietary intake, which have potential to shift the microbiome balance between methanogens and sulfur-degrading methanotrophic bacteria, can assist to stabilise mood.

Another embodiment contemplated by the present disclosure includes an educational resource for clinicians involving, for example, an interactive website or traditional teaching devices for educating clinicians (e.g. general practitioners, psychiatrists, other medical professionals and counsellors) concerning the novel methods of diagnosing and managing psychosis on the basis of MTHFR genotype and accessory biochemical tests, sensory processing tests and education around appropriate doses and monitoring of patients based upon, for example, riboflavin treatment.

All publications mentioned in this specification are herein incorporated by reference. The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

It will be appreciated by persons skilled in the art that numerous variations and/or modifications may be made to the invention without departing from the spirit or scope of the invention as broadly described. The present embodiments are, therefore, to be considered in all respects as illustrative and not restrictive.

The present invention will now be described with reference to the following specific examples, which should not be construed as in any way limiting the scope of the invention.

### EXAMPLES

### Example 1—General Methodologies

### Participant Recruitment

This study was approved by the Queen Elizabeth Hospital Research Ethics Committee (No: 2009139) and protocols and methods conformed to that committee's regulatory standards. Participants were assessed at the Queen Elizabeth Hospital and the Basil Hetzel Institute for Translational Health at Woodville, South Australia and two satellite psychiatric clinics in the Western Adelaide community catchment area.

Recruitment of patients with schizophrenia and schizoaffective disorder and controls lacking these disorders was from multi-ethnic backgrounds in an age-range between 18 and 60 years. The aim of recruitment was to impose sufficient exclusion criteria to minimise confounding variables and strip psychosis in the case sample as far as possible down to its bare functional form. In this way, potential confounding effects of substance abuse, organic causes and medication were minimised and candidate markers which have strong discrimination and case-detection efficacy for functional psychosis could be exposed and be matched for age and sex with control participants.

Similarity of psychotic symptoms occurs in both schizophrenia and schizoaffective conditions and their diagnoses were made with roughly equal incidence in the project setting at a 1.2 to 1 ratio, respectively. Recruitment and assessments were conducted directly and by phone across hospital, research institute and community clinic settings. Control participants were recruited by phone from a volunteer population associated with the hospital and drawn from the same catchment area as patients. Non-detained ward patients in partial remission but with residual symptoms of psychosis, were recruited and assessed in the expected last week of their admission, by which time they had stabilised pharmacotherapy and were sufficiently recovered to give informed consent. After 7 early drop-outs due to declining mental state, a total of 82 symptomatic participants (cases) were recruited and completed assessment. Early statistical analysis of confounders required that 15 participants on SSRI or SNRI antidepressant medication were excluded from further analysis, due to their suppressive effect on catecholamine levels, therefore the number of patient participants used in the final statistical analysis was 67. To minimise severity bias by comparison of very-sick with well-controls in this case—control study, ward patients were recruited within a week of their expected discharge back into the community from which controls were drawn and selection bias was minimised by recruiting some clinic patients by phone and assessing them in the same community setting as the recruited controls.

Together with a review of the participant's case notes, a standard interview protocol collected demographic information and information related to development, organic, biochemistry and sensory-processing disorder. Also recorded was absence or presence of developmental difficulty or learning delay, medical co-morbidity, and head injury, family history of mental illness, glasses or hearing aids. The Brief Psychiatric Rating Scale (BPRS) and the Positive and Negative Syndrome Scale for Schizophrenia (PANS S), were amalgamated in the interest of reducing assessment time. Using this rating tool, each symptom was rated from 1 to 7 for intensity. These ratings were then summated to give an overall symptom-intensity-rating (SIR) index, which was taken as a second measure of clinical severity. Hospital and clinic ratings were made by psychiatrically trained registrars, who were blind to index laboratory and sensory-processing test results, but not to patient status, at the time of rating. Ratings were checked by DSM diagnostic checklist. Rating for control participants were made by one psychiatrically-trained assessor. It was not considered practical in the real world, for raters to be blinded to the diagnostic status of the participant, since many patients were unable to mask their condition, due to residual psychotic symptoms. After determining visual and hearing acuity, all participants were assessed for selected sensory and cognitive variables related to auditory and visual processing.

Assessments were conducted in auditory and visual domains, at a time separated from both blood and urine collection and within two hours of such biological sample-collection. Where applicable, visual assessment was conducted using the participant's usual glasses and alternate-cover-test was conducted prior to visual testing, to exclude visual fixation disparity (phoria or tropia) as a potentially confounding variable. Visual assessments included near and distance visual acuity, visual attention span, speed and accuracy of visual processing. Auditory assessments were conducted in a quiet room (ambient noise level 20 dB) and preceded by examination of the external auditory meatus to exclude obvious pathology or sebum obstruction. Audiometry examination was conducted using the MAICO Audiogram MA 40 [22], at 250 Hz to 4000 Hz to determine hearing deficits (defined as air-bone conduction gaps >10 Hz and/or sensory threshold abnormalities >500-1000 Hz) and laterality differences. Auditory processing assessment outcome measures were of acuity, attention, and threshold speed and accuracy of auditory processing. All assessments were performed by a neuro-psychiatrically trained assessor who was blind to laboratory results. It was considered impractical to blind the assessor to participant status, as residual symptoms of psychosis were obvious to the trained observer. A total of 72 control participants, were recruited with the assistance of the Population Research and Outcomes Studies (PROS) Unit of the University of Adelaide.

These participants were volunteers, from the same catchment area as patients, affiliated with the Department of Medicine and the North-West Adelaide Health Study (NWAHS), for research purposes. Using the same exclusion protocol as for patients, these participants were age-stratified and randomly recruited by phone contact, over the same assessment period as patients. In a low prevalence condition such as schizophrenia with a period prevalence 0.35 per 1000 in the Australian population (Saha S et al 2005). Control participants were not excluded on the basis of a family history of mental illness or schizophrenia though none reached threshold for schizophrenia or any DSM diagnosable mental illness. Controls were nevertheless rated for their subclinical symptoms by a psychiatrically-trained assessor who was not blind to their asymptomatic status, but was blind to all biological test results at the time of rating. Five presenting control participants were assessed, but had their data excluded due to their not meeting basic vision or hearing criteria for participation in the study. The control sample used for the final analysis was drawn from the same catchment area and outpatient environment as the patients and the sample for final analysis consisted of 67.

Antipsychotic medication remained stable during the assessment period and DSM IV-R criteria case diagnoses were made by trained staff and checked by consensus opinion and DSM IV-R symptom-checklist. Patients and controls were rated for clinical and sub-clinical symptoms respectively, and had biological samples taken prior to auditory and visual processing assessments. Control participants were randomly letter and phone-recruited from participants in the North West Adelaide catchment area after age and sex-stratified based upon patient recruitment data. Similar exclusion criteria as for patient recruitment were imposed and though no control participants possessed a diagnosis of mental illness, they were rated for subclinical symptoms in a similar manner to case participants.

Exclusion criteria included medication with Clozapine, Olanzapine which are frequently-prescribed medications for ward and outpatient clinic patients with repeated admissions for psychosis. Together with antihistamines, the following medications have prominent histamine-binding effects, so were excluded as histamine was a candidate biomarker. Patients taking antipsychotic-agents such as Zuclopenthixol, Modecate, Amisulpride, Quetiapine and Risperidone were included. Persons on mood stabilizing medications were allowed. Persons with active or unremitted use of alcohol or other substance abuse were excluded, since this can confound neurotransmitter results. Persons with organic cerebral damage as evidenced by a clinically documented, investigated or descriptive history of hospitalized head injury, unconsciousness or central nervous system disease were also excluded, as were persons with upper respiratory tract infections, middle ear congestion or known sensory or learning disability. Persons with extra-pyramidal signs in ocular, arm or hand muscles were excluded prior to consent. Persons receiving vitamin therapy were also excluded due to the inclusion of vitamins as candidate markers. It was not possible to exclude smoking and have any chance of patient recruitment. Subjects taking anti-histamine medication or vitamin supplementation similar to candidate markers were excluded.

Specific inclusion and exclusion criteria are set out below:

| Inclusion Criteria | Method |
|---|---|
| Orientated in time, place and person. | Clinical examination and case- |
| Non-detained under Mental Health Act | note examination |
| Capacity to consent | |
| Diagnosis of schizophrenia or schizoaffective | American Psychiatric Association: Diagnostic |
| disorder, made by a consultant psychiatrist in | and Statistical Manual of Mental Disorders. |
| the ward or community satellite clinic setting, | (4^{th} Ed. Rev) (DSM IV-R). American |
| according to the DSM-IV-R classification | Psychiatric Association 1994. |
| criteria and checked against DSM-IV-R | Washington DC. 17. Simpson GM, Angus |
| symptom | JWS. A rating scale for extrapyramidal side |
| checklist at the recruitment stage | effects. Acta Psychiatry Scand. 1970; 212 |
| | (44): 11-9. |
| Absence of rigidity, dyskinesia, tremor or | Clinical examination: Simpson GM, Angus |
| instability in ocular-muscle dysfunction, hand, | JWS. (1970) A rating scale for |
| forearm and shoulder. | extrapyramidal side effects. Acta |
| | Psychiatrica Scandinavica, 212 (44), 11-19. |
| Zuclopenthixol, Modecate, Amisulpride | Treatment chart and case-note examination |
| Risperidone, Quetiapine, sodium valproate. | |
| Risk factors of interest: such as family history | Case-note examination and personal inquiry. |
| of mental illness, history of emotional or | |
| physical abuse, developmental disorder, | |
| learning disorder or subclinical head injury. | |

| Exclusion Criteria | Method |
|---|---|
| Lack of orientation in time, place and | Clinical examination and case-note examination |
| person. | |
| Detained under Mental Health Act | |
| Lacking consent capacity | |
| Alternative diagnosis | American Psychiatric Association: Diagnostic and |
| | Statistical Manual of Mental Disorders. (4^{th} Ed. Rev) |
| | (DSM IV-R). American Psychiatric Association 1994. |
| | Washington DC. 17. Simpson GM, Angus JWS. A |
| | rating scale for extrapyramidal side effects. Acta |
| | Psychiatry Scand. 1970; 212 (44): 11-9. |
| Unstable pharmacotherapy over | Treatment chart and case-note examination |
| assessment period. | |
| Extra-pyramidal side effects in | Clinical examination; Simpson GM, Angus JWS. |
| ocular, hand, forearm and shoulder | (1970) A rating scale for extrapyramidal side effects. |
| | Acta Psychiatrica Scandinavica, 212 (44), 11-19. |
| Clozapine, Olanzapine, anti- | Treatment chart, case-note examination and personal |
| histamines or vitamin therapy | questioning. |
| Recent or unresolved history or | Treatment chart, case-note examination and personal |
| positive test for alcohol or other | questioning |
| substance abuse. | |
| Upper respiratory tract infections | Treatment chart, case-note examination and personal |
| | questioning |
| Intellectual disability or | Treatment chart, case-note examination and personal |
| clinicallydocumented or descriptive- | questioning. |
| history of head injury with | |
| unconsciousness or hospitalisation. | |
| Visual fixation disparity (phoria or | Alternate cover test. Riordan-Eva P. Cunningham Jr. |
| tropia) | ET. Vaughan & Asbury's General Ophthalmology. |
| | 18th ed. Lange Medical Book. McGraw-Hill; 2011. |
| | New York: |
| Visual and hearing acuity disability | Case-note examination and personal questioning |
| | Sussex Vision test of near vision. Near vision test |
| | card SNT-3000-L, 2009-2011. Snellen H, 1860, |
| | ‘Snellen Chart’, Sussex Vision International Ltd. |
| | Maico Diagnostics: Operating Instructions MA 40, |
| | Diagnostic GmbH, 2005 Salzufer 13/14 D-10583, |
| | Berlin, Germany. Available from: |
| | www.audiometrics.net/resources/MA40E.pdf |

Recruitment resulted in a highly-characterized group of participants and final data analysis was based on data from 67 cases and 67 control participants. The sample characteristics and details of catchment area characteristics, recruitment process outcomes, and participant medications can be found below and in Tables 1 to 3.

TABLE 1

| | | | | Schizoaffective | | | | | | | | Total | |
|---|---|---|---|---|---|---|---|---|---|---|---|---|---|
| | | Schizophrenia | | psychosis | | Psychosis FI | | Cases | | Controls | | Participants | |
| | | n | % | n | % | n | % | n | % | n | % | n | % |
| Sex | Female | 12 | 36.4% | 16 | 53.3% | 2 | 50.0% | 30 | 44.8% | 34 | 50.7% | 64 | 47.8% |
| | Male | 21 | 63.6% | 14 | 46.7% | 2 | 50.0% | 37 | 55.2% | 33 | 49.3% | 70 | 52.2% |

| | | | | | | | | | | | | |
|---|---|---|---|---|---|---|---|---|---|---|---|---|
| Persons | 33 | 100.0% | 30 | 100.0% | 4 | 100.0% | 67 | 100.0% | 67 | 100.0% | 134 | 100.0% |

TABLE 2

| | | | | Schizoaffective | | | | | | | | | | | | Total | | |
|---|---|---|---|---|---|---|---|---|---|---|---|---|---|---|---|---|---|---|
| Character- | Schizophrenia | | | Psychosis | | | Psychosis FI | | | Cases | | | Controls | | | Participants | | |
| istics | n | Mean | SE | n | Mean | SE | n | Mean | SE | n | Mean | SE | n | Mean | SE | n | Mean | SE |
| Age | 33 | 40.7 | 2.0 | 30 | 40.8 | 1.9 | 4 | 37.3 | 7.4 | 67 | 40.5 | 1.3 | 67 | 45.7 | 1.4 | 134 | 43.1 | 1.0 |
| Age of onset | 31 | 23.9 | 2.0 | 27 | 22.5 | 1.5 | 3 | 32.7 | 10.1 | 61 | 23.7 | 1.3 | 0 | | | 61 | 23.7 | 1.3 |
| Duration of | 31 | 16.5 | 1.9 | 27 | 18.0 | 2.3 | 3 | 8.0 | 1.0 | 61 | 16.8 | 1.4 | 0 | | | 61 | 16.8 | 1.4 |
| Illness (DOI) | | | | | | | | | | | | | | | | | | |
| Symptom | 33 | 113.8 | 5.7 | 30 | 94.4 | 5.7 | 4 | 119.5 | 14.8 | 67 | 105.4 | 4.0 | 67 | 42.8 | .3 | 134 | 74.1 | 3.4 |
| Intensity | | | | | | | | | | | | | | | | | | |
| Rating (SIR) | | | | | | | | | | | | | | | | | | |
| Body Mass | 23 | 31.1 | 2.3 | 27 | 29.6 | 1.4 | 3 | 25.8 | 1.2 | 53 | 30.0 | 1.2 | 66 | 26.7 | .6 | 119 | 28.2 | .6 |
| Index (BMI) | | | | | | | | | | | | | | | | | | |
| Right hand | 32 | 92.1 | 3.0 | 29 | 94.5 | 2.4 | 4 | 82.5 | 17.5 | 65 | 92.6 | 2.1 | 67 | 93.1 | 1.7 | 132 | 92.8 | 1.3 |
| dominance % | | | | | | | | | | | | | | | | | | |
| Urine | 33 | 8.8 | .9 | 29 | 9.3 | 1.2 | 4 | 12.3 | 5.1 | 66 | 9.2 | .7 | 67 | 9.5 | .7 | 133 | 9.3 | .5 |
| Creatinine | | | | | | | | | | | | | | | | | | |
| (mmol/L) | | | | | | | | | | | | | | | | | | |
| 5-Hydroxyl | 33 | 4.6 | 1.4 | 29 | 3.3 | .8 | 4 | 9.5 | 4.8 | 66 | 4.3 | .8 | 67 | 1.6 | .1 | 133 | 2.9 | .4 |
| indole acetic | | | | | | | | | | | | | | | | | | |
| acid (5- | | | | | | | | | | | | | | | | | | |
| HIAA) | | | | | | | | | | | | | | | | | | |
| Plasma | 33 | 10.5 | .4 | 29 | 9.5 | .5 | 4 | 9.3 | 1.4 | 66 | 10.0 | .3 | 66 | 9.5 | .3 | 132 | 9.7 | .2 |
| homocysteine | | | | | | | | | | | | | | | | | | |
| (umol/L) | | | | | | | | | | | | | | | | | | |
| Red cell | 29 | 38.3 | .9 | 29 | 41.6 | 1.2 | 3 | 37.3 | 1.9 | 61 | 39.8 | .7 | 67 | 39.6 | .7 | 128 | 39.7 | .5 |
| acetylcholine | | | | | | | | | | | | | | | | | | |
| esterase | | | | | | | | | | | | | | | | | | |
| (U/gb Hb) | | | | | | | | | | | | | | | | | | |
| Hearing | 31 | 548.4 | 27.0 | 23 | 587.0 | 67.8 | 3 | 500.0 | 0.0 | 57 | 561.4 | 30.8 | 60 | 550.0 | 19.5 | 117 | 555.6 | 18.0 |
| Threshold | | | | | | | | | | | | | | | | | | |
| (Db) | | | | | | | | | | | | | | | | | | |
| Visual | 32 | 6.5 | .6 | 26 | 6.0 | .7 | 3 | 8.0 | 3.0 | 61 | 6.3 | .5 | 67 | 5.2 | .1 | 128 | 5.7 | .2 |
| threshold of | | | | | | | | | | | | | | | | | | |
| near vision | | | | | | | | | | | | | | | | | | |

TABLE 3
Condition type

| (STATA analysis) | Schizophrenia | Schizoaffective | Other | Total |
|---|---|---|---|---|
| ABILIFY | 1 | 1 | 0 | 2 |
| AMISULPRIDE | 1 | 1 | 0 | 2 |
| CHLORPROMAZI | 1 | 1 | 0 | 2 |
| FLUPENTHIXOL | 1 | 1 | 0 | 2 |
| HALOPERIDOL | 0 | 1 | 0 | 1 |
| LITHIUM | 0 | 3 | 0 | 3 |
| BENZODIAZEPIN | 2 | 2 | 0 | 4 |
| PALIPERIDONE | 1 | 0 | 0 | 1 |
| QUETIAPINE | 2 | 8 | 0 | 10 |
| RISPERIDONE | 18 | 7 | 3 | 28 |
| VALPROATE | 2 | 2 | 0 | 4 |
| ZIPRAZIDONE | 1 | 1 | 0 | 2 |
| ZUCLOPENTHIX | 4 | 1 | 0 | 5 |
| MODECATE | 0 | 1 | 0 | 1 |
| OTHER | 1 | 9 | 1 | 11 |
| Total | 35 | 39 | 4 | 78 |
| Cases | 29 | 28 | 4 | 61 |
| Valid cases: | 61 | | | |
| Missing cases: | 6 | | | |

### Biochemical Marker Analysis

Standardized collection methods, protocols and citations are documented in Tables 4 and 5 below. Blood was assayed by commercial laboratories for vitamin B6, vitamin B12, red cell folate, plasma homocysteine, serum copper, serum ceruloplasmin, red cell zinc, serum histamine, methyltetrahydrofolate reductase (MTHFR 677 C->T) gene polymorphism, and vitamin D. Urine assays were used for determining levels of creatinine, dopamine (DA), noradrenaline (NA), adrenaline (AD) and two of their metabolites (homovanillic acid (HVA), methoxy-hydroxymandelic acid (MHMA)) as well as the serotonin metabolite 5-hydroxyindoleacetic acid (5-HIAA) and the oxidative stress biomarker hydroxyhemopyrroline-2-one (HPL).

TABLE 4

| | All fasting blood samples, collected between 9 | |
|---|---|---|
| | and 11 am daily, transported directly with no | |
| Nutrition-Biochemistry | storage | Laboratory/Reference |
| Vitamin D (25-OH) | Diasorin Liason assay kit, for use on the | Clinpath Laboratories |
| | Liaison platform. (nmol/L) | |
| Serum total Vitamin B12 | Competitive Electrochemiluminescent | Clinpath Laboratories |
| | Immunoassay. Roche Modular E 170 | |
| | Automated Immunoassay Analyser, using | |
| | Roche Vitamin B12 Reagent. (nmol/L) | |
| Plasma Red Cell Folate | Competitive Electrochemiluminescent Protein | Clinpath Laboratories |
| | Binding Assay, using Roche Modular E 170, | |
| | using Roche Folate Red Blood Cell (RBC) | |
| | Reagent and Roche Folate RBC Haemolysing | |
| | Reagent on Automated Immunoassay Analyser. | |
| Serum Vitamin B6 | Whole blood High Pressure Liquid | Sullivan Nicolaides Pathology |
| (Pyridoxal-5′-phosphate | Chromatography with fluorescent detection. | |
| coenzyme form) | Chromsystems Vitamin B6 in Whole Blood High | |
| | pressure Liqid Chromatography Reagent Kit. | |
| | Waters Alliance 2695 Separations Module. | |
| Serum Copper | Flame Atomic Absorption Spectrophotometry. | Douglass Hanly Moir Pathology |
| | Varian AA-240F5. (umol/L) | |
| Plasma Red Cell Zinc | Inductively coupled plasma mass spectroscopy | Sullivan Nicolaides Pathology. |
| Serum Ceruloplasmin | Immunoturbidimetric method, using 6K91-30 | Douglass Hanly Moir Pathology. |
| Percentage Free Copper/Red | Percentage of free copper in the serum | Calculated |
| Cell Zinc | calculated by an equation based on the molecular | |
| | and atomic weights of ceruloplasmin and copper | |
| | (one ceruloplasmin molecule binds to six copper | |
| | atoms). The ratio of the percentage free copper | |
| | to red cell zinc was calculated as “percentage free | |
| | copper”/“Red cell zinc umol/L”. | |
| Intermediate substrates | | |
| and enzymes | | |
| MTHFR Ala222Val (C677T) | Real time PCR analysis | Douglass Hanly Moir Pathology. |
| methyl tetrahydrofolate | Roche Diagnostics Light-Cycler 480 kit. Using | |
| reductase polymorphism | TecnoBiol reagents, Sigma probes and primers on | |
| | Roche LC480 analyser. | |
| Plasma homocysteine | Ice transported EDTA sample. Competitive | SA Pathology |
| | Chemiluminescent Immunoassay, using Seimens | |
| | Homocysteine reagent on Seimens Advia centaur | |
| | Automated Immunoassay.(umol/L) | |
| Serum histamine | Beckman Coulter Radio Immunoassay, using | Sullivan and Nicolaides. |
| | Beckman Coulter R.I.A. Kit on Perkin Elmer | |
| | Wizard 1470 Automated Gamma Counter. | |
| | (umol/L) | |

TABLE 5

| Test | Method, Analyzer, Reagents. | Laboratory/Reference |
|---|---|---|
| Neurotransmitters | | |
| Biogenic amines: | Spot-baseline (fasting) urinary | SA Pathology, Adelaide, South Australia. |
| Dopamine, | neurotransmitter testing (second void | |
| Noradrenaline and | morning), snap-frozen to minus 30 degrees | Whiting MJ. 2009. Simultaneous measurement |
| Adrenaline, | and analysed by mass spectrometry, using | of urine metanephrines and catecholamines by |
| | nanomols per millimol of urinary creatinine as | liquid chromatography with tandem mass |
| | a standard. | spectrometric detection. Annals of Clinical |
| | | Biochemistry, 46:129-136 |
| Creatinine | Spot urine specimen from the same void as | SA Pathology, Adelaide SA. |
| | biogenic amines, expressed in (millimols per | |
| | Litre) | |
| Oxidative stress: | Fasting urine sample collected whilst patient at | |
| Urinary | rest, separated from blood drawing by | |
| hydroxyhemopyrroline- | minimum of 2 hours | |
| 2-one | | |

### Visual and Auditory Processing Assessments

Methods for assessing and measuring visual and auditory processing parameters are detailed below.
Near vision acuity (Sussex Vision International Ltd. http://sussexvision.co.uk/index.php/near-tests/reading-tests.html): Sussex Vision test of near vision. Near vision test card SNT-3000-L, 2009-2011
Visual (symbol) span (Based on Visual Symbol Span subset test of WMS-IV (Weschler 2009) Wechsler Memory Scale—WMS-IV A&NZ Language Adapted Edition); https://www.pearsonclinical.com.au/products
/view/212): Increasing number of symbols are presented in a standardised order from left to right. Test score reported as the absolute number of visual symbols.
Distance vision (Binocular distance vision acuity) (The Snellen-Chart): Right distance vision, then left distance vision, with 20 seconds inter-test interval
Threshold visual speed of processing performance as a percentage of age (expresses visual processing speed in terms of the visual processing system's relative age) (Brain Boy Universal Professional instrument, MediTECH 2010; http://www.meditech.de/fileadmin/download/anleitungen/manual_BUP-neu-03.03.2010.indd-mail.pdf): Subject sees two brief flashes of light randomly presented from left-to-right or right-to-left on multiple occasions, and must decide which light flash appeared first. The inter-stimulus time interval (ISI) between the flashes is shortened by computer algorithm, if the answer is correct, otherwise it is lengthened. A performance-age rating, is provided, configured against norms-for-age. Performance-age is subtracted from the subject's actual age and the result divided by the age of the test subject is multiplied by 100. Shortest interval of time a subject can notice between the order of presentation of two optical stimuli. Speed of visual order processing increases with age. For adults between the range of 18 and 60 years, the normal range for visual speed of processing is 24 to 72 milliseconds). For adults between the range of 18 and 60 years, the normal range for visual speed of processing is 24 to 72 milliseconds.
Reverse digit span—measures auditory (verbal) working memory (Subset of Wechsler Adult Intelligence Scale III (Wechsler 1997) http://www.personclinical.com/psychology/products/100000243/wechsler-adult-intelligence-scale--third-edition-wais-iii.html). With gaze aversion by listening subject and tester, digits are read in set sequence. The subject is asked to repeat them in reverse order. Reported as the absolute number of digits correctly recalled in reverse order. Normal range is 6 to 7.
Competing words performance for age as a percentage of age—Intra-cerebral dichotic listening performance for processing of auditory information (SCAN-3:A Tests for Auditory Processing Disorders in Adolescents and Adults (Keith 2009); https://www.pearsonclinical.com.au/produc ts/view/315): A voice-over CD and earphones test ability to correctly identify both of two competing-words (CW), delivered separately to the right and left ears. Using this test's normative-for-age database, the difference between each subject's expected and actual performance-for-age was calculated, and this was then divided by the actual age of the test subject, and multiplied by 100. Normal range varies with age.
Threshold speed of Auditory processing—Speed of auditory processing systems relative to age (Brain Boy Universal Professional instrument (MediTECH 2010): Subject hears two clicks, randomly presented from right to left and left to right side, presented through headphones. By pressing a right or left button, a decision must be made from which side the dual-stimulus originates. If the answer is correct, the inter-stimulus interval between flashes (ISI) is shortened, otherwise it is lengthened. The auditory order threshold is the shortest ISI a subject can correctly differentiate between two auditory impressions. A read-out of the threshold speed of auditory (order) processing is provided, along with a norm performance-age rating. Auditory speed of (order) processing performance as a percentage of age is calculated by subtracting the norm-for-age from the performance-age, divided by the age of the test subject, multiplied by 100. Speed of auditory processing reduces with age. For adults in the age range of 18 and 60 years, the normal range for auditory speed of processing is 46 to 72 milliseconds.

### Statistical Analysis

Sample characteristic analysis, including characteristics related to medication and risk factors were examined using XLSTAT and STATA software. Receiver Operating Characteristic (ROC) analysis was carried out using STATA software (STATA SE Version 13.1. Stata Corp LP) and IPSS software (IBM SPSS Statistics version 20, IBM Corporation), for each MTHFR 677 variant. Spearman's correlation analysis for continuous and/or ROC variables was also determined for each MTHFR 677 variant to determine diagnostic linkage and diagnostic threshold level and marker type. Logistic regression analysis for prediction of risk of case-ness or other functional outcome for reach MTHFR 677 variant, determined diagnostic probability and/or risk and.or likelihood of diagnosis, with sensitivity, specificity, positive predictive value (PPV) and negative preditive value (NPV) percentages and values, respectively. Mann Whitney non parametric U test compared differences between case and control groups for continuous variables. Odds ratio analysis was also conducted as described hereinbelow. All correlations and predictions were at the 95% level of confidence.

An AUC of 0.5-0.7 represents poor discrimination, 0.7-0.8 acceptable discrimination, 0.8-0.9 excellent discrimination, and >0.9 out-standing discrimination. A high sensitivity means that a test only rarely misses classifying a person with schizophrenia/psychosis as having such diagnosis, and therefore, the test has utility as a diagnostic method. A high specificity means that a test only rarely designates a person with schizophrenia/psychosis as being free of that diagnosis, so the test therefore has utility as a diagnostic exclusion, screening tool. Sensitivity and specificity are considered acceptable at ≥85% and ideal at ≥90%. Missing data were imputed using STATA.

### Example 2—Initial Biomarker Analysis for MTHFR 677 Variants

The MTHFR gene was selected as a marker for examination in this study because this gene codes for the MTHFR enzyme which is the rate-limiting factor in the methylation cycle. In the normal form of this gene, cytosine is at position 677, leading to an alanine at amino acid 222. However when there is thymidine at position 677, there is a valine substitution at amino acid 222 and this homozygous form of the gene (TT) encodes a thermolabile enzyme with reduced activity compared to individuals with the CC or CT (heterozygous) forms of the gene. Although there is ethnic variability related to this polymorphism, ten percent of the North American population are T-homozygous for this polymorphism.

The data set from this study was into three data-sets based upon the three possible MTHFR C677T variants (wild type (CC), heterozygous (CT) and homozygous (TT) types) in order to examine key correlates within those three data sets. For the purpose of MTHFR 677 C-T polymorphism analysis in relationship to biophenotype, pooled data from all participants was split according to MTHFR 677 C->T variant. Though there were only 7 participants in the study with the homozygous form of the MTHFR 677 TT genotype, comparison of the data sets still yielded variables of significance and correlative results analysis are presented in Tables 6 and 7 below.

TABLE 6
MTHFR 677 Split-sample correlates for psychosis case-ness and functional measures, at 95% CI.

| | MTHFR TT | | | | MTHFR CC | | | | MTHFR CT | | | |
|---|---|---|---|---|---|---|---|---|---|---|---|---|
| | Variable | N | Rho | P | Variable | N | Rho | P | Variable | N | Rho | P |
| Psychosis | HPL/creat | 7 | 0/723 | 0.067 | 5-HIAA | 65 | 0.475 | 0.000 | NA/MHMA | 60 | 0.545 | 0.000 |
| case-ness | Free % Cu/Zn | 7 | 0.722 | 0.067 | 5-HIAA ROC | 65 | 0.458 | 0.034 | NA/MHMAROC | 60 | 0.537 | 0.000 |
| | High DA ROC | 7 | 0.730 | 0.062 | DA | 65 | 0.263 | 0.019 | AD/MHMA | 60 | 0.427 | 0.001 |
| | High Distance | 7 | 0.750 | 0.052 | High DA ROC | 65 | 0.291 | 0.000 | AD/MHMAROC | 60 | 0.673 | 0.000 |
| | Vision R | | | | NA | 65 | 0.634 | 0.000 | NA/DA | 61 | 0.498 | 0.000 |
| | Low visual span | 7 | 0.730 | 0.062 | High NA ROC | 65 | 0.643 | 0.000 | NA/DA ROC | 61 | 0.481 | 0.000 |
| | ROC | | | | NA/MHMA | 63 | 0.552 | 0.000 | AD/NA ROC | 61 | 0.298 | 0.020 |
| | 27/42 symptoms | | | | High NA/MHMA ROC | 65 | 0.477 | 0.000 | % freeCu/ZnROC | 62 | 0.297 | 0.029 |
| | 1/6 sensory | | | | AD | 65 | 0.669 | 0.000 | Low folate ROC | 62 | 0.323 | 0.011 |
| | processing | | | | AD/NA | 65 | 0.361 | 0.003 | High vitamin B12 ROC | 62 | 0.239 | 0.062 |
| | deficits | | | | High AD ROC | 65 | 0.603 | 0.000 | HPL/creatinine | 61 | 0.380 | 0.003 |
| | | | | | AD/MHMA | 63 | 0.634 | 0.000 | DA | 61 | 0.384 | 0.002 |
| | | | | | High Ad/MHMA ROC | 63 | 0.650 | 0.000 | High DA ROC | 61 | 0.449 | 0.000 |
| | | | | | HPL/creat | 65 | 0.330 | 0.007 | NA | 61 | 0.586 | 0.000 |
| | | | | | HPL/Creat ROC | 65 | 0.292 | 0.018 | High NA | 61 | 0.647 | 0.000 |
| | | | | | HPL/SG | 61 | 0.286 | 0.026 | AD | 61 | 0.591 | 0.000 |
| | | | | | RC Folate | 64 | −0.245 | 0.051 | High AD ROC | 61 | 0.607 | 0.000 |
| | | | | | Low RC folate ROC | 64 | 0.288 | 0.021 | VSOP age add % | 60 | 0.654 | 0.000 |
| | | | | | Vitamin B6 | 63 | −0.335 | 0.007 | Low vis span ROC | 61 | 0.640 | 0.000 |
| | | | | | Low B6 ROC | 63 | 0.432 | 0.000 | Lowreversedig span | 61 | 0.562 | 0.000 |
| | | | | | Vitamin D | 64 | −0.344 | 0.005 | CW age diff | 58 | 0.553 | 0.000 |
| | | | | | Low vitamin D ROC | 64 | 0.300 | 0.016 | ASOP age diff % | 58 | 0.689 | 0.000 |
| | | | | | VSOP age add % ROC | 57 | 0.697 | 0.000 | 41/42 symptoms | | | |
| | | | | | Low visual span ROC | 48 | 0.656 | 0.000 | 5/6 sensory processing | | | |
| | | | | | ASOP age diff % ROC | 58 | 0.547 | 0.000 | deficits | | | |
| | | | | | CW agediff ROC | 60 | 0.504 | 0.000 | | | | |
| | | | | | Low reverse digit span | 60 | 0.394 | 0.002 | | | | |
| | | | | | Low distance visionR ROC | 60 | 0.342 | 0.007 | | | | |
| | | | | | Diabetes | | | | | | | |
| | | | | | 38/42 symptom, 6/6 sensory | 61 | 0.231 | 0.073 | | | | |
| | | | | | processing deficits | | | | | | | |
| Symptom | DA | 7 | 0.749 | 0.053 | 5-HIAA | 65 | 0.403 | 0.001 | high DA ROC | 61 | 0.263 | 0.041 |
| Intensity | HPL/creat | 7 | 0.768 | 0.044 | DA | 65 | 0.230 | 0.065 | NA | 61 | 0.428 | 0.001 |
| Rating | Free Cu/Zn | 7 | 0.729 | 0.063 | High DA ROC | 65 | 0.498 | 0.000 | High NA ROC | 61 | 0.446 | 0.000 |
| (SIR) | High Free | 7 | 0.676 | 0.096 | NA | 65 | 0.585 | 0.000 | NA/MHMA | 61 | 0.418 | 0.001 |
| | Cu/Zn ROC | | | | High NA ROC | 63 | 0.452 | 0.000 | High NA/MHMAROC | 61 | 0.418 | 0.001 |
| | NO NA/NHMA | 7 | 0.000 | 1.000 | NA/MHMA | 65 | 0.392 | 0.001 | AD | 61 | 0.510 | 0.000 |
| | ROC | | | | High NA/MHMA ROC | 65 | 0.508 | 0.000 | AD/NA | 61 | 0.281 | 0.028 |
| | Low histamine < | 7 | 0.676 | 0.096 | AD | 65 | 0.471 | 0.000 | High AD ROC | 61 | 0.466 | 0.000 |
| | 0.035 | | | | AD/NA | 65 | 0.304 | 0.014 | AD/MHMA | 60 | 0.440 | 0.000 |
| | Distance vision | 7 | 0.717 | 0.065 | High AD ROC | 63 | 0.508 | 0.000 | High AD/MHMAROC | 60 | 0.365 | 0.004 |
| | on R | | | | AD/MHMA | 63 | 0.554 | 0.000 | HPL/Creat | 61 | 0.416 | 0.001 |
| | 20/27 symptoms | | | | High AD/MHMA ROC | 65 | 0.341 | 0.005 | HPL/SG | 65 | 0.224 | 0.073 |
| | 1/6 sensory | | | | HPL/Creat | 65 | 0.253 | 0.042 | P1 Homocysteine | 61 | 0.271 | 0.035 |
| | processing | | | | HPL/creat ROC | 61 | 0.371 | 0.021 | Se B12 | 62 | 0.310 | 0.035 |
| | disorder | | | | HPL/SG | 64 | −0.288 | 0.003 | High Se B12 ROC | 62 | 0.320 | 0.011 |
| | | | | | Vitamin D | 64 | 0.252 | 0.045 | RC Folate | 62 | 0.258 | 0.043 |
| | | | | | Low vit D ROC | 64 | 0.217 | 0.086 | Low RC Folate ROC | 62 | 0.262 | 0.039 |
| | | | | | Low RC Folate ROC | 57 | 0.697 | 0.000 | Low vit D ROC | 6 | 0.234 | 0.069 |
| | | | | | VSOP age add % ROC | 58 | 0.571 | 0.003 | Free Cu/Zn | 62 | 0.236 | 0.065 |
| | | | | | Low visual span ROC | 58 | 0.495 | 0.000 | LowreversedigitROC | 61 | 0.488 | 0.000 |
| | | | | | ASOP age doff ROC | 60 | 0.435 | 0.001 | CW diff ROC | 58 | 0.563 | 0.000 |
| | | | | | CW diff ROC | 60 | 0.381 | 0.003 | ASOP age diff % ROC | 58 | 0.607 | 0.000 |
| | | | | | Distance vision R | 60 | 0.276 | 0.032 | HighDistancevisionR | 61 | 0.238 | 0.027 |
| | | | | | Low reversedigitspan ROC | | | | 4/6 SP deficits | | | |
| | | | | | 6/6 sensory processing | | | | 40/42 symptoms (95%) | | | |
| | | | | | deficits | | | | | | | |
| | | | | | 39/42 symptoms of psychosis | | | | | | | |
| | | | | | (93%) | | | | | | | |
| Global | AD/NA | 7 | 0.841 | 0.018 | 5HIAA | 58 | −0.483 | 0.000 | 5HIAA | 58 | −0.296 | 0.024 |
| Assess- | DA | 7 | −0.852 | 0.015 | 5HIAA ROC | 58 | −0.487 | 0.000 | DA | 58 | 0.509 | 0.000 |
| ment of | Free Cu/Zn | 7 | −0.741 | 0.057 | DA | 58 | −0.301 | 0.019 | High DA ROC | 58 | −0.346 | 0.008 |
| Function | Urine creatinine | 7 | −0.704 | 0.077 | High DA ROC | 58 | −0.387 | 0.003 | NA | 58 | −0.500 | 0.000 |
| (GAF) | Significantly | | | | NA | 58 | −0.522 | 0.000 | High NA ROC | 58 | −0.575 | 0.000 |
| | protective | | | | High NA ROC | 58 | −0.544 | 0.000 | NA/MHMA | 58 | −0.584 | 0.000 |
| | CW diff ROC | | | | NA/MHMA | 58 | −0.622 | 0.000 | HighNA/MHMA ROC | 57 | −0.534 | 0.000 |
| | 1/6 sensory | | | | AD | 58 | −0.622 | 0.000 | AD | 58 | −0.563 | 0.000 |
| | deficits | | | | AD/NA | 58 | −0.412 | 0.001 | High AD ROC | 58 | −0.550 | 0.000 |
| | 17/42 (41%) | | | | High AD ROC | 58 | −0.531 | 0.000 | AD/MHMA | 58 | −0.487 | 0.000 |
| | symptoms | | | | AD/MHMA | 57 | −0.618 | 0.000 | HighAD/MHMA ROC | 57 | −0.339 | 0.002 |
| | | | | | HPL/creat | 57 | −0.297 | 0.054 | HPL/Creat | 58 | −0.362 | 0.006 |
| | | | | | HPL/SG | 58 | −0.254 | 0.024 | High HPL/Creat ROC | 58 | −0.360 | 0.006 |
| | | | | | RC folate | 57 | +0.407 | 0.002 | RC Folate | 58 | −0.348 | 0.007 |
| | | | | | Low RC Folate ROC | 57 | −0.425 | 0.001 | Low RC Folate ROC | 59 | 0.319 | 0.015 |
| | | | | | Vitamin B6 | 56 | +0.273 | 0.042 | Low visual span ROC | 59 | −0.342 | 0.008 |
| | | | | | Low vit B6 ROC | 56 | −0.346 | 0.009 | VSOP age add % | 59 | −0.342 | 0.008 |
| | | | | | Vitamin D | 57 | +0.297 | 0.025 | High DV on | 58 | −0.708 | 0.000 |
| | | | | | Low vit D ROC | 57 | −0.265 | 0.046 | CW diff ROC | 57 | −0.521 | 0.000 |
| | | | | | VSOP age add % ROC | 50 | −0.635 | 0.000 | Lowreversdigitspan | 57 | −0.507 | 0.000 |
| | | | | | Low vis span ROC | 51 | −0.591 | 0.000 | 5/5 Sensory processing | 55 | −0.589 | 0.000 |
| | | | | | CW diff ROC | 53 | −0.509 | 0.000 | deficits | 58 | −0.449 | 0.000 |
| | | | | | ASOP age diff# | 51 | −0.471 | 0.000 | symptoms of psychosis | | | |
| | | | | | Low reverse digit span ROC | 53 | −0.368 | 0.007 | 42/42(100%) | | | |
| | | | | | Distance vision R ROC | 53 | −0.291 | 0.034 | | | | |
| | | | | | 6/6 sensory processing | | | | | | | |
| | | | | | deficits | | | | | | | |
| | | | | | All Sx psychosis 37/42 | | | | | | | |
| | | | | | (88%) | | | | | | | |
| Duration | High DA ROC | 7 | 0.698 | 0.081 | 5-HIAA | 58 | 0.509 | 0.000 | 5 HIAA | 60 | 0.241 | 0.063 |
| of Illness | HPL/creat | 7 | 0.808 | 0.028 | 5-HIAA ROC | 60 | 0.475 | 0.000 | DA | 60 | 0.241 | 0.008 |
| DOI | HPL/Creat ROC | 7 | 0.676 | 0.096 | DA | 60 | 0.304 | 0.018 | High DA ROC | 60 | 0.393 | 0.002 |
| | Low DV R | 7 | 0.717 | 0.070 | High DA ROC | 60 | 0.304 | 0.018 | NA | 60 | 0.570 | 0.000 |
| | Low visual span | 7 | 0.698 | 0.081 | NA | 60 | 0.661 | 0.000 | High NA ROC | 60 | 0.605 | 0.000 |
| | Marginally | | | | High NA ROC | 60 | 0.673 | 0.000 | NA/MHMA | 60 | 0.530 | 0.000 |
| | related | | | | NA/MHMA | 58 | 0.554 | 0.000 | HighNA/MHMA ROC | 59 | 0.335 | 0.006 |
| | to visual SP | | | | High NA/MHMA ROC | 60 | 0.499 | 0.000 | AD | 60 | 0.579 | 0.000 |
| | deficits | | | | AD | 60 | 0.667 | 0.000 | High AD ROC | 60 | 0.601 | 0.000 |
| | Related to 64% | | | | High AD ROC | 60 | 0.624 | 0.000 | AD/MHMA | 59 | 0.428 | 0.001 |
| | symptoms of | | | | AD/MHMA | 57 | 0.613 | 0.000 | HighAD/MHMA ROC | 61 | 0.663 | 0.000 |
| | psychosis | | | | High AD/MHMA ROC | 58 | 0.608 | 0.000 | HPL/Creat | 60 | 0.361 | 0.005 |
| | 27/42 | | | | HPL/Creat | 60 | 0.306 | 0.017 | HPL/Creat R | 60 | 0.415 | 0.001 |
| | | | | | High HPL/creat ROC | 60 | 0.262 | 0.043 | Se B12 | 61 | 0.268 | 0.037 |
| | | | | | HPL/SG | 60 | 0.260 | 0.045 | High Se B12 ROC | 61 | 0.279 | 0.030 |
| | | | | | Vitamin B6 | 58 | −0.368 | 0.004 | RC Folate | 61 | 0.258 | 0.045 |
| | | | | | Low vit B6 ROC | 58 | 0.375 | 0.004 | Low RC Folate ROC | 61 | 0.280 | 0.029 |
| | | | | | Vitamin D | 60 | −0.343 | 0.007 | Free Cu/Zn | 61 | 0.215 | 0.096 |
| | | | | | Low vit D ROC | 60 | 0.285 | 0.027 | High Free Cu/Zn ROC | 60 | 0.247 | 0.057 |
| | | | | | Low RC Folate ROC | 59 | 0.252 | 0.054 | Low visual span ROC | 60 | 0.684 | 0.000 |
| | | | | | Low visual span ROC | 55 | 0.673 | 0.000 | ASOP agediff % RO | 57 | 0.646 | 0.000 |
| | | | | | VSOP age add % ROC | 54 | 0.672 | 0.000 | VSOP age add % ROC | 59 | 0.639 | 0.000 |
| | | | | | ASOP age diff % ROC | 55 | 0.487 | 0.000 | CW diff ROC | 57 | 0.573 | 0.000 |
| | | | | | CW diff ROC | 57 | 0.466 | 0.000 | LowreversdigspanROC | 60 | 0.507 | 0.000 |
| | | | | | HighDistance Vision R ROC | 57 | 0.361 | 0.006 | LowdistancevisionRR | 60 | 0.403 | 0.001 |
| | | | | | Low reverse digit span ROC | 57 | 0.386 | 0.003 | 6/6 sensory processing | | | |
| | | | | | 6/6 SP deficits | | | | deficits | | | |
| | | | | | 88% symptoms (37/42) | | | | 98% symptoms 41/42 | | | |
| | | | | | including suicidality n 60, | | | | | | | |
| | | | | | rho 0.593, P 0.000 | | | | | | | |
| Social and | Nil | | | | DA | 58 | −0.283 | 0.031 | | | | |
| Occupa- | | | | | NA | 58 | −0.593 | 0.000 | | | | |
| tional | | | | | AD/NA | 58 | −0.288 | 0.028 | | | | |
| Assess- | | | | | RC Folate | 58 | −0.287 | 0.029 | | | | |
| ment | | | | | HPL/creat | 58 | −0.261 | 0.048 | | | | |
| Scale | | | | | Vitamin D6 | 56 | 0.364 | 0.006 | | | | |
| SOFAS | | | | | Vitamin D | 57 | 0.255 | 0.055 | | | | |

TABLE 7
MTHFR 667 split-sample inter-variable correlates for psychosis biochemistry, at 95% CI.

| | MTHFR Polymorphism | | | | | | | | | | | |
|---|---|---|---|---|---|---|---|---|---|---|---|---|
| | MTHFR homozygous | | | MTHFR heterozygous | | | MTHFR negative | | | Total | | |
| Demographics | | | Std. | | | Std. | | | Std. | | | Std. |
| of Variables | Mean | N | Deviation | Mean | N | Deviation | Mean | N | Deviation | Mean | N | Deviation |
| 5-HIAA | 1.57 | 7 | .787 | 2.26 | 61 | 3.371 | 3.71 | 65 | 6.269 | 2.93 | 133 | 4.985 |
| NA/DA | .18 | 7 | .064 | .18 | 61 | .112 | .18 | 65 | .155 | .18 | 133 | .133 |
| AD/NA | .13 | 7 | .048 | .18 | 61 | .154 | .17 | 65 | .120 | .17 | 133 | .134 |
| NA/MHMA | 1978.57 | 7 | 1237.557 | 1481.11 | 60 | 751.035 | 1646.30 | 63 | 1017.576 | 1587.95 | 130 | 917.675 |
| % Free Cu/Zn | .14 | 7 | .440 | .29 | 62 | .465 | .26 | 64 | .585 | .27 | 133 | .523 |
| AD/MHMA | 2.50 | 7 | 1.893 | 2.70 | 60 | 2.796 | 2.89 | 63 | 2.990 | 2.78 | 130 | 2.838 |
| Vitamin B12/vit D | 8.99 | 7 | 4.953 | 9.77 | 61 | 7.796 | 9.17 | 64 | 5.778 | 9.44 | 132 | 6.717 |
| Vit B12/folate | .2604 | 7 | .086 | .2504 | 62 | .147 | .2255 | 64 | .101 | .2390 | 133 | .124 |
| Vit B12/zinc | 33.0879 | 7 | 10.160 | 31.1095 | 62 | 14.153 | 28.9488 | 65 | 12.429 | 30.1647 | 134 | 13.132 |
| Vit B12/[zinc × folate] | .0191 | 7 | .007 | .0184 | 62 | .010 | .0169 | 64 | .008 | .0177 | 133 | .009 |
| [Vit B12 × % free Cu]/ | .0271 | 7 | .105 | .0641 | 62 | .112 | .0503 | 64 | .173 | .0555 | 133 | .144 |
| Zn × folate | | | | | | | | | | | | |
| [Vitamin B12 × % free Cu]/ | .0002 | 7 | .001 | .0008 | 59 | .001 | .0007 | 62 | .004 | .0007 | 128 | .003 |
| [Zn × folate × Vitamin B6] | | | | | | | | | | | | |
| [Vitamin B12 × % free Cu × | .0068 | 7 | .024 | .0073 | 58 | .014 | .0025 | 62 | .053 | .0049 | 127 | .038 |
| homocysteine]/ | | | | | | | | | | | | |
| [zinc × folate × vit B6] | | | | | | | | | | | | |
| Plasma Homocysteine | 10.87 | 7 | 4.816 | 9.64 | 61 | 1.869 | 9.70 | 64 | 2.426 | 9.74 | 132 | 2.359 |
| Histamine | .61 | 7 | .273 | .69 | 62 | .304 | .67 | 65 | .381 | .68 | 134 | .340 |
| Vitamin D | 59.00 | 7 | 21.510 | 52.43 | 61 | 20.015 | 51.78 | 64 | 24.280 | 52.46 | 132 | 22.152 |
| Red cell folate | 1830.71 | 7 | 484.247 | 1794.52 | 62 | 517.123 | 1779.00 | 64 | 374.541 | 1788.95 | 133 | 448.715 |
| Serum vitamin B12 | 452.14 | 7 | 126.796 | 421.65 | 62 | 205.263 | 386.43 | 65 | 154.087 | 406.16 | 134 | 178.572 |
| Vitamin B6 | 315.00 | 7 | 554.519 | 146.86 | 59 | 124.183 | 115.04 | 63 | 87.609 | 140.45 | 129 | 164.688 |
| HPL/CREAT | 2.27 | 7 | 1.590 | 4.37 | 61 | 5.601 | 5.04 | 65 | 6.517 | 4.59 | 133 | 5.947 |
| HPL/SG | 15.91 | 7 | 12.948 | 34.15 | 61 | 41.714 | 45.50 | 65 | 79.182 | 38.74 | 133 | 62.437 |
| Symptom Intensity | 82.29 | 7 | 57.113 | 74.10 | 62 | 38.347 | 73.25 | 65 | 38.205 | 74.11 | 134 | 39.088 |
| rating (SIR Index) | | | | | | | | | | | | |
| Global Assessment | 71.43 | 7 | 33.877 | 70.34 | 59 | 27.083 | 67.76 | 58 | 26.806 | 69.19 | 124 | 27.143 |
| of function (GAF) | | | | | | | | | | | | |

### Example 3—Psychosis-Related Symptoms Associated with MTHFR C677T Variants

All MTHFR C677T variants contribute to case-identification, illness severity, duration of illness and disability in schizophrenia and schizoaffective psychosis. Approximately 49% of participants presented with the wild-type MTHFR CC polymorphism and demonstrated low-methylation biochemistry indicated by low flavin, high oxidative stress, low folate, low vitamin D and B6 and a tendency to high histamine and high 5HIAA. This phenotype related to symptoms of with low visual span ROC, ASOP age diff % ROC, CW diff ROC, low reverse digit span, low distance vision on the right ROC, judgement and insight impairment, delusions, unusual thought content, suspiciousness, cognitive disorganization, emotional withdrawal, blunted affect, thought preoccupation, poor rapport, passivity/apathy, poor attention, hostility, excitement, abstract thinking impairment, lack of spontaneous conversation, social avoidance, anxiety, un-cooperativeness, hallucinations, bizarre behavior, disrupted volition, distractibility, self-neglect, depressed mood, poor impulse control, tension, motor retardation, suicidality, grandiosity, guilt, ideas reference and control, motor hyperactivity, somatic concern, elated mood, history of abuse, disorientation, mannerism and posturing, stereotypic thinking and/or blank periods.

Approximately 46% of subjects had the heterozygous MTHFR CT polymorphism and also display symptoms similar to those with the wild type MTHFR CC polymorphism and additionally demonstrated elevated homocysteine and vitamin B12 in relationship to vitamin D. 5% of participants had the homozygous MTHFR TT variant and had a good-methylation biochemical profile (with elevated free copper:zinc ratio, lesser oxidative stress, sufficient vitamin B6, D and folate levels and a tendency to low histamine and no 5HIAA). This phenotype may be protective against 64% of the typical symptoms of psychosis and related to high distance vision on the right, low visual span ROC, blunted affect, distractibility, hallucinations, hostility, anxiety, somatic concern, tension, delusions, judgement and insight impairment, excitement, grandiosity, poor impulse control, thought preoccupation, unusual thought, depressed mood, poor rapport, emotional withdrawal, social avoidance, passivity/apathy, self-neglect, lack of spontaneity. conversation, uncooperativeness, bizarre behavior, elated mood, motor hyperactivity, and/or sense of outside self.

Core biomarker findings identified within each MTHFR 677C->T gene variant related to diagnostic case-ness, duration of illness (DOI) and functional measures of Global Assessment of Function (GAF), Symptom Intensity (SIR), Clinical Global Index of Severity (CGI), Social and Occupational Function Scale (SOFAS) and hospital admission frequency as well as for individual symptoms and their biochemical variants which determined subtypes within each of the MTHFR homozygous, heterozygous and no-polymorphism forms of the MTHFR 677C->T gene. Symptoms were then weighted according to their Odds ratio of association to case-ness, DOI and the aforementioned functional measures, in order to provide a scoring index system for prediction of Severity, Disability, likely Duration of Illness and particularly clinically-significant symptoms of suicidality and hostility within the psychosis setting. These novel findings demonstrate that psychosis symptoms are not unitary entities but are differentiated in their spread across three major MTHFR 677C->T genotypes related to biochemistry arising from differently activated pathways.

TABLE 8
Current mental state Spearman's correlates for case-ness and case-related functional measures for the three
MTHFR 677C->T gene variants
Spearman's correlates for case-ness and case-related functional measures for the three MTHFR 677C->T gene variants.

| | MTHFR | | | | |
|---|---|---|---|---|---|
| | homozygous | | | | |
| | Biochemical and | | | | |
| | sensory-processing | | | | |
| | parameters | N | Rho | P | MTHFR heterozygous |
| Homozygous | MTHFR Het +/− | 134 | −0.218 | 0.011 | MTHFR +/−n 134, rho −0.218, P 0.011 |
| MTHFR | MTHFR −/− | 134 | −0.218− | 0.008 | NO MTHFR polymorph n 134, rho −0.901, P 0.000 |
| Polymorphism | HPL ROC | 134 | 0.163 | 0.061 | Suicidality n 134, rho −0.155, P 0.074 |
| and symptoms | Somatic concern | 134 | 0.143 | 0.098 | |
| of dissociation | Outside self | 134 | 0.312 | 0.000 | |
| Psychosis | Other identities | 134 | 0.159 | 0.067 | |
| CASE-NESS | HPL/creat | 7 | 0.720 | 0.067 | DA n 61, rho 0.384, P 0.002 |
| | Free Cu/Zn | 7 | 0.841 | 0.018 | High DA ROC, n 61, rho 0.449, P 0.000 |
| | High DA ROC | 7 | 0.730 | 0.062 | NA n 61, rho 0.586, P 0.000 |
| | NO HIAA | 7 | 0.000 | 1.000 | High NA ROC n 61, rho 0.647, P 0.000 |
| | High Distance Vision R | 7 | 0.750 | 0.052 | NA/MHMA n 60, rho 0.545, P 0.000 |
| | Low Visual span ROC | 7 | 0.730 | 0.062 | High NA/MHMA ROC n 61, rho 0.574, P 0.000 |
| | Blunted Affect | 7 | 0.966 | 0.000 | AD n 61, rho 0.591, P 0.000 |
| | Distractibility | 7 | 0.966 | 0.000 | High AD ROC n 61, rho 0.607, P 0.000 |
| | Hallucinations | 7 | 0.966 | 0.000 | AD/MHMA n 60, rho 0.427, P 0.001 |
| | Hostility** | 7 | 0.966 | 0.000 | High AD/MHMA ROC n 60, rho 0.673, P 0.000 |
| | Anxiety | 7 | 0.966 | 0.000 | HPL/Creat n 61, rho 0.355, P 0.008 |
| | Somatic concern | 7 | 0.966 | 0.000 | High HPL ROC, N 61, rho 0.380, P 0.003 |
| | Tension | 7 | 0.966 | 0.000 | High Free Cu:Zn ROC n 61, rho 0.279, P 0.029 |
| | Delusions | 7 | 0.956 | 0.001 | Free Cu/Zn n 62, rho 0.232, P 0.070 |
| | Judg. & insight impaired | 7 | 0.956 | 0.001 | RC Folate n 62, rho −0.307, P 0.015 |
| | Excitement | 7 | 0.956 | 0.001 | Low RC Folate ROC n 62 rho 0.239, P 0.063 |
| | Grandiosity | 7 | 0.956 | 0.001 | High Se B12 ROC n 62, rho 0.239, P 0.062 |
| | Poor impulse control | 7 | 0.956 | 0.001 | Vit D n 61, rho −0.220, P 0.089 |
| | Suspiciousness | 7 | 0.956 | 0.001 | Low Vit D ROC n 61, rho 0.217, P 0.093 |
| | Thought preoccupation | 7 | 0.956 | 0.001 | VSOP age add % n 60, rho 0.654, P 0.000 |
| | Unusual thought | 7 | 0.720 | 0.068 | Low vis span ROC n 61, rho 0.640, P 0.000 |
| | Depressed mood | 7 | 0.720 | 0.068 | Low reverse digit span n 61, rho 0.562, P 0.000 |
| | Poor Rapport | 7 | 0.720 | 0.068 | CW diff (dichotic listening) ROC n 58, rho 0.553, P 0.000 |
| | Emotional Withdrawal | 7 | 0.720 | 0.068 | ASOP age diff % n 58, rho 0.689, P 0.000 |
| | Social avoidance | 7 | 0.720 | 0.068 | High Distance vision R n 61, rho 0.330, P 0.009 |
| | Passivity/Apathy | 7 | 0.720 | 0.068 | Judgement and Insight impairment n 62, rho 0.932, P 0.000 |
| | Self-neglect | 7 | 0.720 | 0.068 | Suspiciousness n 62, rho 0.883, P 0.000 |
| | Lack spont. conversation | 7 | 0.720 | 0.068 | Cognitive disorganization n 62, rho 0.835, P 0.000 |
| | Uncooperativeness | 7 | 0.720 | 0.068 | Delusions n 62, rho 0.822, P 0.000 |
| | Bizzare behavior | 7 | 0.720 | 0.068 | Unusual thought n 62, rho 0.812, P 0.000 |
| | Elated mood | 7 | 0.720 | 0.068 | Blunted affect n 62, rho 0.790, P 0.000 |
| | Motor hyperactivity | 7 | 0.072 | 0.068 | Poor attention n 62, rho 0.789, P 0.000 |
| | Sense of outside self | 7 | 0.720 | 0.068 | Abstract thinking impairment n 62, rho 0.766, P 0.000 |
| | 27/42 symptoms | | | | Distractibility n 62, rho 0.765, P 0.000 |
| | No abstract thinking | | | | Thought preoccupation n 62, rho 0.743, P 0.000 |
| | impairment, poor | | | | Hallucinations n 62, rho 0.742, P 0.000 |
| | attention, disorientation | | | | Hostility n 62, rho 0.742, P 0.000 |
| | No blank periods, unreal | | | | Poor impulse control n 62, rho 0.724, P 0.000 |
| | feelings, other identities. | | | | Emotional withdrawal n 62, rho 0.720, P 0.000 |
| | No guilt, stereotypic | | | | Anxiety n 62, rho 0.676, P 0.000 |
| | thinking, mannerism or | | | | Excitement n 62, rho 0.674, P 0.000 |
| | posturing, ideas ref and | | | | Passivity/Apathy n 62, rho 0.674, P 0.000 |
| | control, | | | | Uncooperativeness, n 62, rho 0.657, P 0.000 |
| | No disturbed volition, | | | | Grandiosity n 62, rho 0.651, P 0.000 |
| | motor retardation. | | | | Self-neglect n 62, rho 0.651, P 0.000 |
| | No suicidality | | | | Lack spontaneous conversation n 62, rho 0. 629, P 0.000 |
| | | | | | Bizarre behavior n 62, rho 0.628, P 0.000 |
| | | | | | Depressed mood n 62, rho 0.626, P 0.000 |
| | | | | | Poor rapport n 62, rho 0.605, P 0.000 |
| | | | | | Motor hyperactivity n 62, rho 0.586, P 0.000 |
| | | | | | Disturbed Volition n 62, rho 0.583, P 0.000 |
| | | | | | Elated mood, n 62, rho 0.582, P 0.000 |
| | | | | | Social avoidance 0.582, P 0.000 |
| | | | | | Disorientation n 62, rho 0.535, P 0.000 |
| | | | | | Ideas reference and control n 62, rho 0.511, P 0.000 |
| | | | | | Motor retardation n 62, rho 0.486, P 0.000 |
| | | | | | Stereotypic thinking n 62, rho 0.462, P 0.000 |
| | | | | | Somatic concern n 62, rho 0.462, P 0.000 |
| | | | | | Tension 62, rho 0.422, P 0.001 |
| | | | | | Suicidality n 62, rho 0.356, P 0.005 |
| | | | | | Blank Periods, n 62, rho 0.356, P 0.005 |
| | | | | | Unreal feelings n 62, rho 0.327, P 0.010 |
| | | | | | History of abuse n 62, rho 327, P0.010 |
| | | | | | Guilt n 62, rho 0.327, P 0.010 |
| | | | | | Mannerisms and posturing n 62, rho 0.296, P 0.020 |
| | | | | | Outside of self n 62, rho 0.225, P 0.078 |
| | | | | | 41/42 symptoms |
| | | | | | No other identities, |

| | Wild-type | | | | |
|---|---|---|---|---|---|
| | enzyme (No | MTHFR | | | |
| | MTHFR gene | No polymorphism | | | |
| | polymorphism) | Biochemical Parameter | N | Rho | P |
| Homozygous | Wild-type | MTHFR homozygous | 134 | −0.218 | 0.011 |
| MTHFR | enzyme (No | MTHFR heterozygous | 134 | 0.901 | 0.000 |
| Polymorphism | MTHFR gene | Suicidality | 134 | −−0.155 | 0.063 |
| and symptoms | polymorphism) | Unreal feelings | 134 | −0.144 | 0.097 |
| of dissociation | | Outside self | 134 | −0.191 | 0.027 |
| Psychosis | Psychosis | DA | 65 | 0.263 | 0034 |
| CASE-NESS | CASE-NESS | High DA ROC | 65 | 0.291 | 0.019 |
| | | NA | 65 | 0.634 | 0.000 |
| | | High NA ROC | 65 | 0.643 | 0.000 |
| | | NA/MHMA | 63 | 0.552 | 0.000 |
| | | High NA/MHMA ROC | 65 | 0.477 | 0.000 |
| | | AD | 65 | 0.669 | 0.000 |
| | | High AD ROC | 65 | 0.603 | 0.000 |
| | | AD/MHMA | 63 | 0.634 | 0.000 |
| | | High Ad/MHMA ROC | 63 | 0.552 | 0.000 |
| | | HPL/creat | 65 | 0.330 | 0.007 |
| | | HPL/Creat ROC | 65 | 0.292 | 0.018 |
| | | RC Folate | 64 | −0.245 | 0.051 |
| | | Low RC folate ROC | 64 | 0.288 | 0.021 |
| | | Vitamin B6 | 63 | −0.335 | 0.007 |
| | | Low B6 ROC | 63 | 0.432 | 0.000 |
| | | Vitamin D | 64 | −0.344 | 0.005 |
| | | Low vitamin D ROC | 64 | 0.300 | 0.016 |
| | | VSOP age add % ROC | 57 | 0.697 | 0.000 |
| | | Low visual span ROC | 48 | 0.656 | 0.000 |
| | | ASOP age diff % ROC | 58 | 0.547 | 0.000 |
| | | CW diff ROC | 60 | 0.504 | 0.000 |
| | | Low reverse digit span | 60 | 0.394 | 0.002 |
| | | Low distance vision R ROC | 60 | 0.342 | 0.007 |
| | | Judgement and Insight | 65 | 0.882 | 0.000 |
| | | impairment | | 0.881 | 0.000 |
| | | Delusions | | 0.859 | 0.000 |
| | | Unusual thought content | | 0.859 | 0.000 |
| | | Suspiciousness | | 0.772 | 0.000 |
| | | Cognitive Disorganization | | 0.772 | 0.000 |
| | | Emotional withdrawal | | 0.765 | 0.000 |
| | | Blunted affect | | 0.751 | 0.000 |
| | | Thought preoccupation | | 0.751 | 0.000 |
| | | Poor rapport | | 0.750 | 0.000 |
| | | Passivity/Apathy | | 0.750 | 0.000 |
| | | Poor attention | | 0.750 | 0.000 |
| | | Hostility | | 0.736 | 0.000 |
| | | Excitement | | 0.730 | 0.000 |
| | | Abstract thinking impairment | | 0.708 | 0.000 |
| | | Lack of spontaneous | | 0.707 | 0.000 |
| | | conversation | | 0.702 | 0.000 |
| | | Social avoidance | | 0.691 | 0.000 |
| | | Anxiety | | 0.690 | 0.000 |
| | | Un-cooperativenes | | 0.686 | 0.000 |
| | | Hallucinations | | 0.686 | 0.000 |
| | | Bizarre behavior | | 0.686 | 0.000 |
| | | Disturbed volition | | 0.664 | 0.000 |
| | | Distractibility | | 0.659 | 0.000 |
| | | Self-neglect | | 0.646 | 0.000 |
| | | Depressed mood | | 0.642 | 0.000 |
| | | Poor impulse control | | 0.556 | 0.000 |
| | | Tension | | 0.556 | 0.000 |
| | | Motor retardation | | 0.510 | 0.000 |
| | | Suicidality | | 0.489 | 0.000 |
| | | Grandiosity | | 0.489 | 0.000 |
| | | Guilt | | 0.486 | 0.000 |
| | | Ideas reference and control | | 0.446 | 0.000 |
| | | Motor hyperactivity | | 0.442 | 0.000 |
| | | Somatic concern | | 0.393 | 0.001 |
| | | Elated mood | | 0.362 | 0.003 |
| | | History of abuse | | 0.276 | 0.026 |
| | | Disorientation | | 0.244 | 0.050 |
| | | Mannerism and posturing | | 0.217 | 0.083 |
| | | Stereotypic thinking | | | |
| | | Blank periods | | | |
| | | 38/42 symptom, 6/6 sensory | | | |
| | | processing deficits | | | |
| | | No unreal feelings or other | | | |
| | | identities | | | |

### Example 4—Urine Analysis of Riboflavin and its Metabolites

Phosphorylation of riboflavin maintains its metabolic trapping in tissues where it is mostly enzyme bound as flavoproteins like FMN and FAD. Dietary and bacterially synthsised riboflavin is continually being distributed across the brushborder membrane of the intestine and urinary metabolites reflect bacterial synthesis and degradation activity in the gastrointestinal tract. Unbound flavins are rapidly hydrolyzed to free riboflavin, which diffuses from cells and is excreted in the urine as riboflavin or other metabolites, such as 7-hydroxymethylriboflavin (7-α-hydroxyriboflavin) and lumiflavin. There is a linear relationship between urinary recovery of riboflavin and its metabolites and riboflavin absorption (bioavailability) and urinary riboflavin recovery can serve as an index of riboflavin absorption (West D W, Owen E C 1969. The urinary excretion of metabolites of riboflavine by man. Brit. J. Nutrition. 23:889-898).

High performance liquid chromatography (HPLC) with fluorometric detection of the eluted riboflavin peak (1, 2) was used herein to analyse urine from subjects. Riboflavin standard was from Sigma Aldrich. Stock riboflavin standard solution (1 mg/mL) was prepared in 10% acetonitrile and stored in −80° C. freezer. The stock solution was diluted periodically as needed from which a new standard curve freshly made for each batch. Working solution with range between range between 4-20 mg/L, was prepared monthly from the stock and store in fridge. Standard curve was obtained by independently diluted levels of riboflavin from one stock solution, and the resulting peak area were then plotted against riboflavin concentrations.

15-50 ml urine was collected in the early morning after overnight fast and immediately frozen at −20° C. and stored at this temperature with protection from light. 170 thawed urine was added to 30 μL of acetonitrile in a 1.5 mL Eppendorf tube and incubated at 4° C. for 30 mins. The tube was then centrifuged at 13000 rpm, 10° C. for 10 mins. The supernatant was transferred into a 96-well plate (Greiner, Australia), and 5 μL were injected on to HPLC to be analysed.

The HPLC used Agilent 1260 Infinity system equipped with Agilent ZORBAX Eclipse Plus C18 column (RRHD 3.0×50 mm, 1.8 μm) with guard column with a flow rate of 0.5 mL/min. The chromatographic separation was performed at 25° C. and used gradient elution with mobile phase A consisting of 1% acetonitrile and B of 50% acetonitrile. The gradient started with 0% B and ramped up to 100% at 2 mins and maintained for 0.9 mins. The mobile phase was then equilibrated to initial condition (at 3 min) before the next injection. Riboflavin was measured with a fluorescence detector (λ_{Ex}: 445 nm and λ_{Em}: 530 nm). Samples with a very high riboflavin level (N=2) required extra dilution. Agilent OpenLAB was used for instrument control and data analysis.

Riboflavin was detected at 3.35 mins. Two ranges of fractions were obtained which were separated at 2 mins. The first range (at 0.4 mins to 1.5 mins) resembles riboflavin metabolites according to the literature using similar chromatographic conditions, including 7-7α-hydroxyriboflavin (Gatuatis et al 1981, Clin Chem 27 (10): 1672-1675).

The first elution peak on HPLC riboflavin urine analysis (Peak 1) represents riboflavin metabolites. The second peak (Peak 2) represents riboflavin. Peaks 1 and 2 were measured as either amplitude or area under the peaks, from which the ratio of Peak 1/Peak 2 was determined. Peak 2 greater than Peak 1 is indicative of riboflavin synthesis. Peak 1 greater than Peak 2 is indicative of riboflavin degradation. All Peak data was also analysed in relationship to creatinine excretion levels. Analysis was also conducted by ROC, Spearman's correlation and logistic regression for each MTHFR 677 variant.

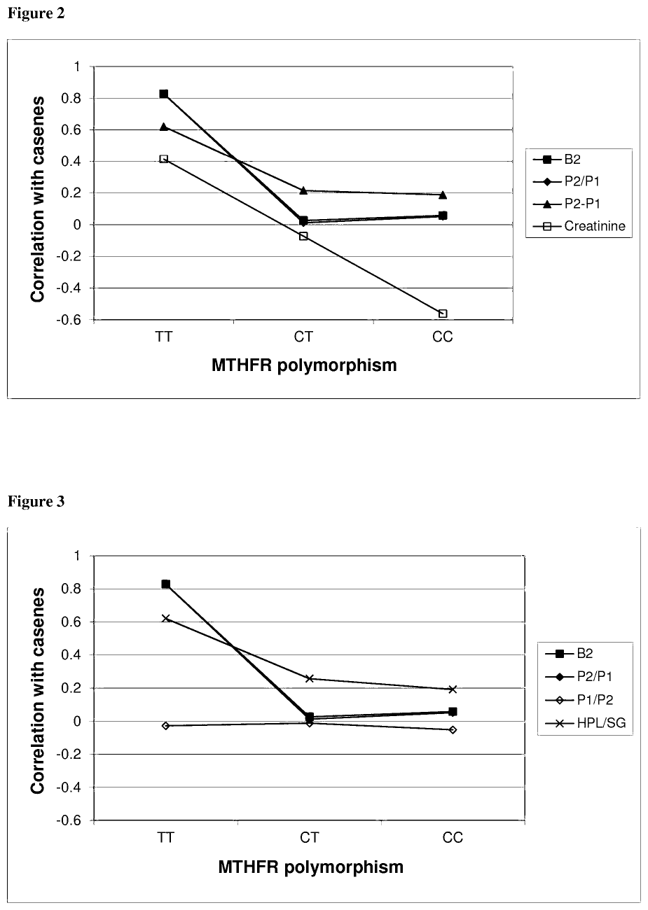

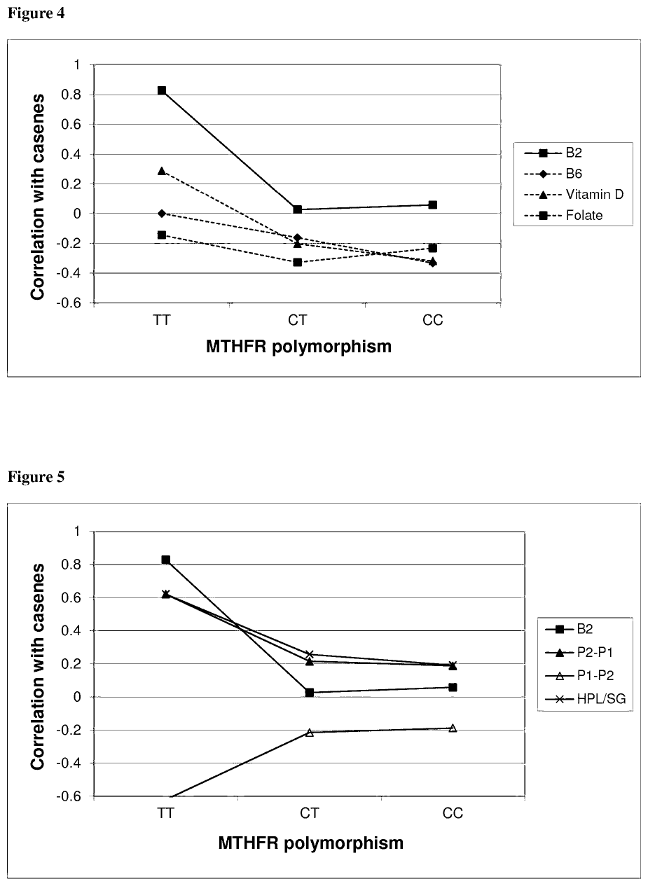

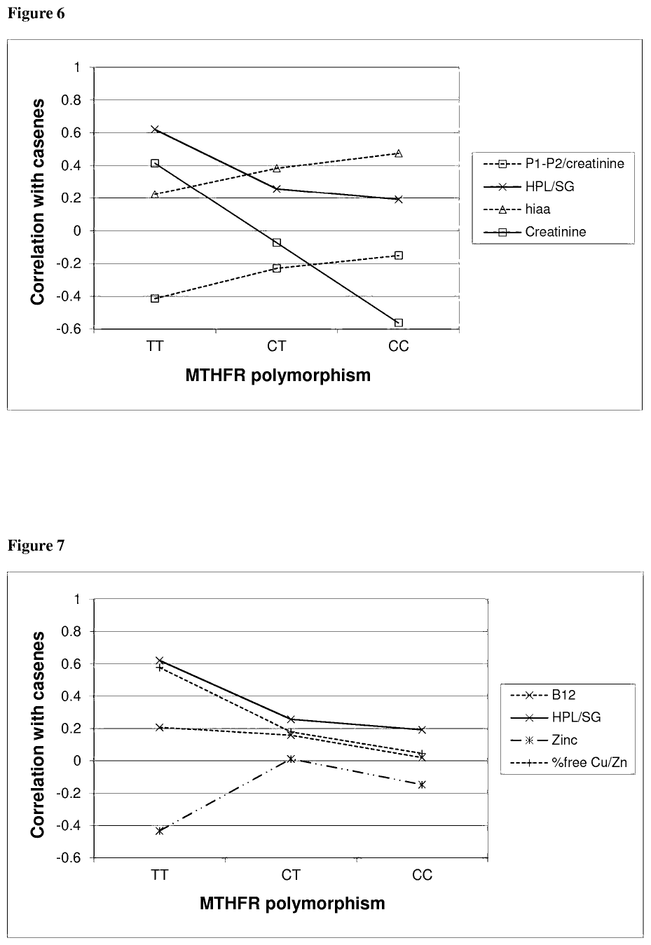

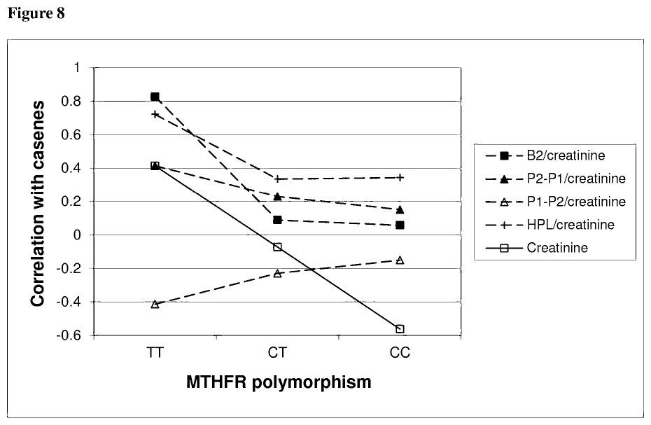

FIGS. 2 to 8 illustrate increased levels of vitamin B2, vitamin B6, vitamin B12, vitamin D, HPL and creatinine excretion, also increased % free copper/Zinc with reduced zinc level, in the MTHFR 677 TT variant, consistent with an overmethylation profile. FIGS. 2 to 8 also illustrate increased levels of 5HIAA (representing degradation of trapped L tryptophan), with lower levels of folate, vitamin B2, vitamin B6, vitamin B12 and vitamin D in the MTHFR 677 CC variant, consistent with functional vitamin degradation and inactivation in an undermethylating setting.

### MTHFR 677 TT Variant

Case-ness is positively correlated with elevated vitamin B2 parameters and these are, as expected, highly correlated with P2/P1 parameters. Despite case-ness and P1/P2, P1-P2 parameters not holding any significant correlation for this variant, vitamin B2 is seen to still hold significant negative correlates with Peak 1 (riboflavin degradation) parameters (P1-P2 N 6, rho 0.771, P 0.072) in a context where it holds a marginal positive correlates with HPL/SG (n7, rho 0.679, P 0.094). This nexus of findings suggest that vitamin B2 and elevated HPL are somehow related and it is noted that they have the distinction of holding significant correlates with the SIR index for high symptom intensity. In this setting of high overall riboflavin relationship to case-ness, but low relationship of riboflavin degradation-product (P1/P2 and P1-P2) to both B2 and HPL, it is noted that sensory processing components of competing words age diff (dichotic listening performance) and visual working memory performance both possess positive correlates, even in the setting of elevated P1 parameters. In keeping with the lesser number of biomedical and sensory processing deficits found in the MTHFR 677 TT variant, Global Assessment of Function (GAF) and Social and Occupational Functioning Scale (SOFAS) outcome measures hold positive correlates, demonstrating conserved functional ability and social and occupational ability associated with this polymorphism. Corresponding negative correlates occur with DOI CGI and SIR, indicating better prognosis along with important reduction in frequency of hospital admissions related to the this MTHFR variant. The conclusion is therefore that a preponderance of riboflavin (P2) and also of its direct P1 metabolites (P1) hold the key to improved sensory function and functional disability outcomes in this homozygous MTHFR 677 TT variant, whereas elevated HPL representing oxidative stress, and over-methylating catecholamine dynamics relate to psychosis case-ness and symptom severity.

Table 9 shows significant correlates (only) for homozygous MTHFR 677 TT variant in relationship to riboflavin and its metabolites and in relationship to sensory impairments and functional outcomes, where P2/P1, P2-P1=riboflavin synthesis and P1-P2 amplitude or area under peak=riboflavin (vitamin B2) degradation products.

TABLE 9
MTHFR 677 TT variant

| | Correlation | | | | | | | | |
|---|---|---|---|---|---|---|---|---|---|
| | Coefficient | | | | | | | | |
| | (rho) Sig. | | | Peak 2 | Peak 2 | Peak 1 | Peak | Peak 1 | Peak 1 |
| MTHFR 677 | (2-tailed) P | | | amplitude/ | area/ | amplitude/ | 1 area/ | amplitude- | area- |
| TT | at 95% Cl | | B2/ | Peak 1 | Peak 1 | Peak 2 | Peak | Peak 2 | Peak 2 |
| homozygous | N = number | B2 | creatinine | amplitude | area | amplituse | 2 area | amplituse | area |
| B2 | rho | | | 0.829 | 0.657 | | | | |
| | P | | | 0.042 | 0.156 | | | | |
| | N | | | 6 | 6 | | | | |
| B2/creat | rho | | | 0.429 | 0.371 | | | | |
| | P | | | 0.019 | 0.005 | | | | |
| | N | | | 6 | 6 | | | | |
| HPL/SG | rho | | | | | | | | |
| | P | | | | | | | | |
| | N | | | | | | | | |
| HPL/creat | rho | | | | | | | | |
| | P | | | | | | | | |
| | N | | | | | | | | |
| B12 | rho | | | | | | | | |
| | P | | | | | | | | |
| | N | | | | | | | | |
| visual span | rho | | | | | .794 | .794 | .853 | |
| | P | | | | | .059 | .059 | .031 | |
| | N | | | | | 6 | 6 | 6 | |
| CW agediff | rho | | | | | | | .900 | |
| | P | | | | | | | .037 | |
| | N | | | | | | | 5 | |
| GAF | rho | | | | | .759 | | | |
| | P | | | | | .080 | | | |
| | N | | | | | 6 | | | |
| DOI | rho | | | | | −.778 | −.845 | | |
| | P | | | | | .069 | .034 | | |
| | N | | | | | 6 | 6 | | |
| DSP | rho | | | | | −.828 | −.828 | | |
| | P | | | | | .042 | .042 | | |
| | N | | | | | 6 | 6 | | |
| SIR | rho | | | | | −0.848 | −.845 | | |
| | P | | | | | 0.034 | .034 | | |
| | N | | | | | 6 | 6 | | |
| SOFAS | rho | | | | | .880 | .880 | | |
| | P | | | | | .021 | .021 | | |
| | N | | | | | 6 | 6 | | |
| CGI | rho | | | | | −.778 | −.778 | | |
| | P | | | | | .069 | .069 | | |
| | N | | | | | 6 | 6 | | |
| Adnit no/DOI | | | | | | −.778 | −.778 | | |
| | rho | | | | | | | | |
| | P | | | | | .069 | .069 | | |
| | N | | | | | 6 | 6 | | |
| | Correlation | | | | | | | | |
| | Coefficient | (Peak 1 | (Peak 1 | | | | | | |
| | (rho) Sig. | amplitude- | area- | | | | | | |
| MTHFR 677 | (2-tailed) P | Peak 2 | Peak 2 | | | Duration | | | |
| TT | at 95% Cl | amplituse)/ | area)/ | | SIR | of illness | | | |
| homozygous | N = number | creatinine | creatinine | case | Index | yrs | ASOP | VSOP | B12 |
| B2 | rho | | | 0.828 | | | | | |
| | P | | | 0.042 | | | | | |
| | N | | | 6 | | | | | |
| B2/creat | rho | | | 0.828 | | | | | |
| | P | | | 0.042 | | | | | |
| | N | | | 6 | | | | | |
| HPL/SG | rho | | | | | | 0.000 | | .679 |
| | P | | | | | | 1.000 | | .094 |
| | N | | | | | | 5 | | 7 |
| HPL/creat | rho | | | 0.722 | .768 | .808 | | | |
| | P | | | 0.067 | .044 | .028 | | | |
| | N | | | 7 | 7 | 7 | | | |
| B12 | rho | | .771 | | | | | 0.9 | |
| | P | | .072 | | | | | 0.037 | |
| | N | | 6 | | | | | 5 | |
| visual span | rho | | | | | | | | |
| | P | | | | | | | | |
| | N | | | | | | | | |
| CW agediff | rho | | | | | | | | |
| | P | | | | | | | | |
| | N | | | | | | | | |
| GAF | rho | | | | | | | | |
| | P | | | | | | | | |
| | N | | | | | | | | |
| DCI | rho | | | | | | | | |
| | P | | | | | | | | |
| | N | | | | | | | | |
| DSP | rho | | | | | | | | |
| | P | | | | | | | | |
| | N | | | | | | | | |
| SIR | rho | | | | | | | | |
| | P | | | | | | | | |
| | N | | | | | | | | |
| SOFAS | rho | | | | | | | | |
| | P | | | | | | | | |
| | N | | | | | | | | |
| CGI | rho | | | | | | | | |
| | P | | | | | | | | |
| | N | | | | | | | | |
| Adnit no/DOI | | | | | | | | | |
| | rho | | | | | | | | |
| | P | | | | | | | | |
| | N | | | | | | | | |

### MTHFR 677 CT

The MTHFR 677 CT heterozygous variant provides an interesting window into riboflavin/biochemistry/sensory interactions as it is characterised by simultaneous effects of its C and T alleles contributing to the dynamic overlapping flux of under and over-biochemistry.

The neuroprotective contribution of the T allele is seen by positive vitamin B2 correlates holding perfectly significant positive correlates with Peak 2 (riboflavin parameters) and also with vitamin B6, whilst vitamin B12 holds interesting inverse correlates with Peak 1 (riboflavin degradation) parameters. In a similar manner to results seen in the TT variant, vitamin B2 and P2 parameters hold strong positive correlates with FMN-activated vitamin B6 whilst together with P1 parameters, vitamin B2 and vitamin D play a protective role in favourably extending reverse digit span (auditory working memory) and conserving dichotic listening (CW) performance, providing the expected inverse correlates for SIR and hospital admission frequency.

In contrast, case-ness in the heterozygous MTHFR 677 CT variant, relates to the contribution of the C allele with negative correlates for folate, vitamin B6 and positive correlates for HPL, whilst high psychosis symptom intensity (SIR), relates to the classic undermethylating phenotype factors of low folate, high vitamin B12 and elevated HPL, with reduced performance on dichotic listening (CW age diff), along with reduced auditory and visual working memory (reversed digit span and visual span) and increased delay in visual and auditory processing speed.

Taken together, findings suggest that vitamin B6, vitamin D and P1 riboflavin metabolites are all somewhat neuroprotective in terms of working memory (reverse digit span) and auditory processing speed (ASOP) and therefore protect against severity and disability. However, in this variant HPL relates inversely to P2 (riboflavin) and appears to also relate adversely to competing words performance (dichotic listening disorder), and notably contributes to case-ness, increased SIR and all adverse functional outcomes as well as hospital admission number and disability pension requirement.

Table 10 shows significant correlates (only) for heterozygous MTHFR 677 CT variant in relationship to riboflavin and its metabolites and in relationship to sensory impairments and functional outcomes, where P2/P1, P2-P1=riboflavin synthesis and P1-P2 amplitude or area under peak=riboflavin (vitamin B2) degradation products.

TABLE 10

| | Correlation | | | | | | | | | | |
|---|---|---|---|---|---|---|---|---|---|---|---|
| | Coefficient | | | | | | | | | | |
| | (rho) | | | | | | | | | (Peak 1 | (Peak 1 |
| | Sig. (2- | | | Peak 2 | Peak 2 | Peak 1 | Peak | Peak 1 | Peak | amplitude- | area- |
| MTHFR 677 | tailed) P at | | | amplitude/ | area/ | amplitude/ | 1 area/ | amplitude- | 1 area- | Peak 2 | Peak 2 |
| CT | 95% Cl | | B2/ | Peak 1 | Peak 1 | Peak 2 | Peak | Peak 2 | Peak | amplituse)/ | area)/ |
| Heterozygous | N = number | B2 | creatinine | amplituse | area | amplitude | 2 area | amplitude | 2 area | creatinine | creatinine |
| B6 | rho | 0.449 | 0.314 | 0.306 | 0.316 | −.306 | −.339 | −.271 | −.245 | | |
| | P | 0.001 | 0.022 | 0.027 | 0.023 | .027 | .012 | .052 | .081 | | |
| | N | 53 | 53 | 52 | 52 | 52 | 54 | 52 | 52 | | |
| B12 | Rho | | | | | −.339 | −.329 | −.301 | −.331 | | |
| | P | | | | | .012 | .015 | .018 | .015 | | |
| | N | | | | | 54 | 54 | 62 | 54 | | |
| B2/creatinine | Rho | | | 0.796 | 0.783 | | | | | | |
| | P | | | 0 | 0 | | | | | | |
| | N | | | 54 | 54 | | | | | | |
| B2 ug/L | rho | | | 0.742 | 0.716 | | | | | | |
| | P | | | 0 | 0 | | | | | | |
| | N | | | 54 | 54 | | | | | | |
| HPL/SG | rho | | | −0.306 | −0.275 | .306 | .275 | .295 | .251 | .415 | .387 |
| | P | | | 0.024 | 0.044 | .024 | .044 | .030 | .067 | .002 | .004 |
| | N | | | 54 | 54 | 54 | 54 | 54 | 54 | 54 | 54 |
| CW agediff | Rho | | | | | | | 0.384 | .366 | .347 | .348 |
| | P | | | | | | | .005 | .008 | .013 | .012 |
| | N | | | | | | | 51 | 51 | 51 | 51 |
| Backward | Rho | | | | | | .278 | .242 | .280 | .295 | .288 |
| digit span | | | | | | | | | | | |
| | P | | | | | | .044 | .081 | .042 | .032 | .037 |
| | N | | | | | | 53 | 53 | 53 | 53 | 53 |
| SIR | rho | | | | | −.275 | −.263 | −.255 | | | |
| | P | | | | | .044 | .055 | .063 | | | |
| | N | | | | | 54 | 54 | 54 | | | |
| DSP | rho | | | | | | | −.347 | −.319 | −.363 | −.324 |
| | P | | | | | | | .011 | .020 | .008 | .018 |
| | N | | | | | | | 53 | 53 | 53 | 53 |

### MTHFR CC Variant

In this variant, case-ness correlates significantly with low vitamin B6, low vitamin D and low folate and the drop in riboflavin levels, the absence of any significant correlate for vitamin B2/creatinine in the presence of strong correlates for under-methylation signatures of elevated NA/MHMA (due to limited availability of FAD-flavin cofactor for MAO enzyme catecholamine metabolism) as well as strong correlates for HPL/creatinine, HPL/SG. Notably, in this variant, HPL/SG relates to case-ness and P1/P2 (riboflavin degradation) parameters (n 61, rho 0.399, P 0.001), in a setting where, of themselves, P1 products are protective of vitamin D levels visual span, auditory working memory and dichotic listening performance. Furthermore, P1 area-P2 area (indicating predominance of riboflavin degradation over riboflavin synthesis), holds positive significant positive correlates for folate (n 57, rho 0.282, P 0.034), vitamin B6 (n 56, rho 0.364, P 0.006) and vitamin D (n 60, rho 0.274, P 0.034), respectively and also holds a significant positive correlate with SOFAS (n 55, rho 0.272, P 0.045), indicating that P1 riboflavin degradation products in themselves have protective, rather than adverse, effects against social and occupational decline.

Table 11 shows significant correlates (only) for homozygous MTHFR 677 CC variant, in relationship to riboflavin and its metabolites and in relationship to sensory impairments and functional outcomes, where P2/P1, P2-P1=riboflavin synthesis and P1-P2 amplitude or area under peak=riboflavin (vitamin B2) degradation products.

TABLE 11

| | Correlation | | | | | | | | |
|---|---|---|---|---|---|---|---|---|---|
| | Coefficient | | | | | | | | |
| | (rho) Sig. (2- | | | Duration | | | | | |
| | tailed) P at | | | of | | | | | |
| MTHFR 677 CC | 95% Cl | | SIR | illness | | | | | |
| (wildtype) | N = number | case | Index | yrs | VitD | RCfolate | B6 | B12 | NA/MHMA |
| case | rho | 1.000 | .846 | .939 | −.344 | −.245 | −.335 | | .552 |
| | P | | .000 | .000 | .005 | .051 | .007 | | .000 |
| | N | 65 | 65 | 60 | 64 | 64 | 63 | | 63 |
| SIR Index | rho | .846 | 1.000 | .753 | −.288 | −.142 | −.148 | | .452 |
| | P | .000 | | .000 | .021 | .264 | .246 | | .000 |
| | N | 65 | 65 | 60 | 64 | 64 | 63 | | 63 |
| Duration of illnes | Rho | .939 | .753 | 1.000 | −.343 | −.206 | −.368 | | .554 |
| | P | .000 | .000 | 0 | .007 | .118 | .004 | | .000 |
| | N | 60 | 60 | 60 | 60 | 59 | 58 | | 58 |
| | rho | 0.33 | | | | | | | |
| HPL_CREAT | P | 0.007 | | | | | | | |
| | N | 65 | | | | | | | |
| | rho | | | | | | | | |
| HPL/SG | P | | | | | | | | |
| | N | | | | | | | | |
| VitD | Rho | −.344 | −.288 | −.343 | 1.000 | .295 | .201 | | −.322 |
| | P | 005 | .021 | .007 | 0 | 019 | .117 | | .011 |
| | N | 64 | 64 | 60 | 64 | 63 | 62 | | 62 |
| RCfolate | rho | −.245 | | | .295 | 1.000 | .287 | .226 | |
| | P | .051 | | | .019 | | .024 | .072 | |
| | N | 64 | | | 63 | 64 | 62 | 64 | |
| B6 | Rho | −.335 | | −.368 | .201 | .287 | 1.000 | .235 | −.227 |
| | P | 007 | | .004 | .117 | 0.24 | | .063 | .078 |
| | N | 63 | | 58 | 62 | 62 | 63 | 63 | 61 |
| NA/MHMA | rho | .552 | .452 | .554 | −.322 | | −.227 | | 1.000 |
| | P | 000 | .000 | .000 | .011 | | 078 | | |
| | N | 63 | 63 | 58 | 62 | | 61 | | 63 |
| B2/creatinine | rho | | | | | | | | |
| | P | | | | | | | | |
| | N | | | | | | | | |
| B2 | rho | .058 | .005 | .028 | −.098 | .005 | .169 | | |
| | P | .659 | .968 | .834 | .454 | .968 | .200 | | |
| | N | 61 | 61 | 57 | 60 | 60 | 59 | | |
| Visual span | rho | | −.629 | | | | | | |
| | P | | 000 | | | | | | |
| | N | | 58 | | | | | | |
| Reverse digit | rho | | −.409 | | | | | | |
| span | | | | | | | | | |
| | P | | .001 | | | | | | |
| | N | | 59 | | | | | | |
| Distance vision V R | rho | | .228 | | | | | | |
| | P | | .080 | | | | | | |
| | | | 60 | | | | | | |
| | N | | | | | | | | |
| VSOP | rho | | .683 | | | | | | |
| | P | | .000 | | | | | | |
| | N | | 57 | | | | | | |
| ASOP | rho | | .432 | | | | | | |
| | P | | .001 | | | | | | |
| | N | | 57 | | | | | | |
| CW age diff | rho | | −.451 | | | | | | |
| | P | | .000 | | | | | | |
| | N | | 60 | | | | | | |

| | Correlation | | | | | | | |
|---|---|---|---|---|---|---|---|---|
| | Coefficient | | | Peak 1 | Peak 1 | Peak 1 | | (Peak 1 |
| | (rho) Sig. (2- | Peak 1 | Peak 1 | amplitude/ | area/ | ampli- | Peak 1 | amplitude- |
| | tailed) P at | amplitude/ | area/ | Peak 2 | Peak 2 | tude- | area- | Peak 2 |
| MTHFR 677 CC | 95% Cl | Peak 2 | Peak 2 | amplituse/ | area/ | Peak 2 | Peak 2 | amplituse)/ |
| (wildtype) | N = number | amplituse | area | creatinine | creatinine | amplituse | area | creatinine |
| case | rho | | | | | | | |
| | P | | | | | | | |
| | N | | | | | | | |
| SIR Index | rho | | | | | | | |
| | P | | | | | | | |
| | N | | | | | | | |
| Duration of illnes | Rho | | | | | | | |
| | P | | | | | | | |
| | N | | | | | | | |
| | rho | | | | | | | |
| HPL_CREAT | P | | | | | | | |
| | N | | | | | | | |
| | rho | 399 | .331 | .326 | | | | |
| HPL/SG | P | .001 | .009 | .010 | | | | |
| | N | 61 | 61 | 61 | | | | |
| VitD | Rho | | | .232 | .274 | .241 | .270 | |
| | P | | | .075 | .034 | .063 | .037 | |
| | N | | | 60 | 60 | 60 | 60 | |
| RCfolate | rho | | | | | | | |
| | P | | | | | | | |
| | N | | | | | | | |
| B6 | Rho | | | | | | | |
| | P | | | | | | | |
| | N | | | | | | | |
| NA/MHMA | rho | | | | | | | |
| | P | | | | | | | |
| | N | | | | | | | |
| B2/creatinine | rho | | | | | | | |
| | P | | | | | | | |
| | N | | | | | | | |
| B2 | rho | | | | | | | |
| | P | | | | | | | |
| | N | | | | | | | |
| Visual span | rho | | | | | .332 | .355 | .266 |
| | P | | | | | .013 | 008 | .050 |
| | N | | | | | 55 | 55 | 55 |
| Reverse digit | rho | | | | | .306 | .277 | .257 |
| span | | | | | | | | |
| | P | | | | | .022 | .039 | .056 |
| | N | | | | | 56 | 56 | 56 |
| Distance vision V R | rho | | | | | | | |
| | P | | | | | | | |
| | N | | | | | | | |
| VSOP | rho | | | | | | | |
| | P | | | | | | | |
| | N | | | | | | | |
| ASOP | rho | | | | | | | |
| | P | | | | | | | |
| | N | | | | | | | |
| CW age diff | rho | | | | | | | |
| | P | | | | | | | |
| | N | | | | | | | |

TABLE 12

| | Correlation | | | | | | |
|---|---|---|---|---|---|---|---|
| MTHFR | Coefficient | | | | | | |
| 677 | (rho) Sig. (2- | | | | | | |
| CC | tailed) P at | | | | | Digit | |
| (wild- | 95% Cl | | Visual | | CW | span | SIR |
| type) | N = number | DV-R6 | span | ASOP | diff | b/w | Index |
| case | Rho | | | | | | |
| | P | | | | | | |
| | N | | | | | | |
| HPL/CREAT | rho | | −.268 | .266 | −.331 | −.284 | .341 |
| | P | | .042 | .046 | .010 | .029 | .005 |
| | N | | 58 | 57 | 60 | 59 | 65 |
| HPL/SG | rho | | | .292 | | | .224 |
| | P | | | .027 | | | .073 |
| | N | | | 57 | | | 65 |
| VitD | rho | | .292 | | | | |
| | P | | .028 | | | | |
| | N | | 57 | | | | |
| RCfolate | rho | | | | .250 | .290 | |
| | P | | | | .055 | .026 | |
| | N | | | | 60 | 59 | |
| B6 | rho | | | | | | |
| | P | | | | | | |
| | N | | | | | | |
| | Correlation | | | | | | |
| MTHFR | Coefficient | | | | | | |
| 677 | (rho) Sig. (2- | | | | | | |
| CC | tailed) P at | | Duration | | | Admit no | |
| (wild- | 95% Cl | | of illness | | | divided by | DSP |
| type) | N = number | GAF | yrs | SOFAS | CGI | DOI | Pension |
| case | Rho | | | | | | |
| | P | | | | | | |
| | N | | | | | | |
| HPL/CREAT | rho | −.297 | .306 | −.287 | .223 | .322 | |
| | P | .024 | .017 | .029 | .092 | .012 | |
| | N | 58 | 60 | 58 | 58 | 60 | |
| HPL/SG | rho | | | | | | |
| | P | | | | | | |
| | N | | | | | | |
| VitD | rho | .297 | | | .255 | −.369 | −.294 |
| | P | .025 | | | .055 | .005 | .023 |
| | N | 57 | | | 57 | 57 | 60 |
| RCfolate | rho | .407 | .282 | −.344 | −.269 | | |
| | P | .002 | .034 | .009 | .040 | | |
| | N | 57 | 57 | 57 | 59 | | |
| B6 | rho | .273 | −.368 | .364 | −.359 | −.382 | −.255 |
| | P | .042 | .004 | .006 | .007 | .003 | .047 |
| | N | 56 | 58 | 56 | 56 | 58 | 61 |

### Conclusions

The data relating to vitamin B2 (Peak 2) and its metabolites (Peak 1) on HPLC analysis, has supported and extended our theoretical understandings of the biochemistry:
that vitamin B2 is unutilised and conserved in the MTHFR 677 TT variant;
that vitamin B2 supports sufficient levels of vitamin B6 activation in the MTHFR 677 TT variant to cofactor multiple methylation and other pathways;
that elevated levels of riboflavin degradation products (P1) in the MTHFR 677 TT variant are neuroprotective in terms of sensory processing and dichotic listening and visual working memory are improved along with functional outcome measures; and
that although HPL elevation relates to case-ness, SIR and duration of illness, in this MTHFR 677 TT variant, it is not related to decline in sensory processing parameters.

The above data demonstrates that in the MTHFR 677 wild-type CC variant vitamin B2 is lower and low vitamin B2 levels relate to low vitamin D, B6 and folate levels. In this setting elevated levels of riboflavin degradation products (P1) are neuroprotective in terms of sensory processing; but elevated levels of HPL are related to reduced sensory processing function and adverse functional outcomes.

Taken together, these results imply that both vitamin B2 and its metabolites are neuroprotective and it is elevated HPL that has potential for adverse affects for sensory processing and functional outcomes. The high correlation between HPL and riboflavin Peak 2 in the homozygous variant is at odds with its relationship with P1 in the wild-type variant and raises the issue of whether HPL can arise from different degradation processes. In line with the theoretical biochemistry, is possible that riboflavin degredation may be F420 facilitated in the undermethylation setting where heme/porphyrin metabolism is stalled because of low vitamin B6, low FAD and Low SAMe. In the MTHFR 677 CC setting, trapped, unmetabolised L-tryptophan is elevated and L tryptophan is known to complex with vitamin B2 in a reaction that forces release of reactive oxygen species. This strongly oxidative reaction may strip off riboflavin's ribytyl side chain and cleave its central ring molecule degrading it to to lumichrome and/or lumiflavin, in a reaction that can be further facilitated by F420. Since HPL formation involves release of reactive oxygen species and formic acid and is the resulting pyrrole degradation fragment, then this could well explain a neurotoxic role for HPL in the MTHFR 677 CC setting.

From examination of the relationship of HPL to riboflavin and its degradation products the inventors theorise that: (i) in the MTHFR 677 CC variant, HPL is an F420 facilitated breakdown product of already-degraded vitamin B2 products which are essentially protective of sensory functioning; however (ii) in the MTHFR 677 CC variant where plentiful FAD, SAMe and vitamin B6 allow free heme synthsis down the porphyrin/heme pathway, HPL may arise either from anaerobic bacterial degradation of vitamin B12's pyrrole (corrin) ring and/or from usual aerobic oxidation pathway of degradation via bilirubin and biliverdin reductase.

### Example 5—MTHFR 677 CC Genotype Indicative of an Under-Methylation Psychosis Phenotype

Based on statistical analysis of biomarker levels in accordance with the above-described examples, the inventors have identified that the presence of the MTHFR 677 CC homozygous variant is associated with an under-methylating phenotype. ROC analysis revealed that this under-methylating psychosis phenotype is associated with one or more of the following:
elevated vitamin B12 divided by vitamin D relative to control values;
elevated homocysteine]/[Zinc X folate X vit B6 X vitamin D], relative to control values;
high AD/MHMA and/or high NA/MHMA ratio levels and/or high AD/MHMA and/or high NA/MHMA ratio levels relative to control values;
low vitamin levels of vitamin B6 levels and/or low levels of vitamin B6 relative to control values;
high histamine levels in association with low vitamin B6 levels;
high histamine levels relative to control values;
elevated urine 5-HIAA levels/values relative to control values;
elevated dopamine multiplied by 5HIAA levels, relative to control levels;
elevated HPL (adjusted for specific gravity HPL/SG) relative to control values;
elevated HPL/creatinine relative to control values;
low red cell folate levels and/or low folate levels relative to control levels/values;
low vitamin D levels and/or low vitamin D levels relative to control values/levels;
low vitamin B6 levels and/or low vitamin b6 levels/values relative to control values/levels;
% free Copper to zinc ratio and/or high % free copper to zinc ratio relative to control values; and/or
absence of significantly elevated urine riboflavin or riboflavin adjusted for creatinine, relative to control values.

TABLE 13

| Variable for | | | | | | OR (odds | OR p | | | |
|---|---|---|---|---|---|---|---|---|---|---|
| MTHFR 677CC | n | Sensitivity | Specificity | PPV | NPV | ratio) | value | AUC | se | P |
| High 5HIAA | 65 | | | | | | | 0.7557 | 0.0553 | 0.0000 |
| High 5HIAAROC | 65 | 84.8% | 59.4% | 0.9% | 99.9% | 8.2 | 0.001 | 0.7211 | 0.0543 | 0.0000 |
| DA x 5HIAA | 65 | | | | | 1.0041 | 0.0270 | 0.7888 | 0.0561 | 0.0000 |
| DA X 5HIAA ROC | 65 | 84.8% | 71.9% | 1.4% | 99.9% | 14.3111 | 0.0000 | 0.7836 | 0.0513 | 0.0000 |
| NA/DA | 65 | | | | | | | 0.8324 | 0.0520 | 0.0000 |
| NA/DA ROC | 65 | 69.7% | 90.6% | 3.3% | 99.8% | 22.2 | 0.000 | 0.8016 | 0.0483 | 0.0000 |
| NA/MHMA | 63 | | | | | | | 0.8187 | 0.0550 | 0.0000 |
| NA/MHMA ROC | 63 | 84.8% | 70.0% | 1.3% | 99.9% | 13.1 | 0.000 | 0.7742 | 0.0531 | 0.0000 |
| Low B6 | 63 | | | | | | | 0.6930 | 0.0672 | 0.0020 |
| Low B6 ROC | 63 | 90.6% | 48.4% | 0.8% | 99.9% | 9.1 | 0.002 | 0.6951 | 0.0526 | 0.0001 |
| vitB12/vit D | 64 | | | | | | | 0.7031 | 0.0644 | 0.0008 |
| High Histamine | 65 | 93.9% | 28.1% | 0.6% | 99.9% | 6.1 | 0.030 | 0.6103 | 0.0456 | 0.0078 |
| ROC | | | | | | | | | | |
| Low Vit D | 64 | | | | | | | 0.6987 | 0.0652 | 0.0012 |
| Low Vit D ROC | 64 | 75.0% | 59.4% | 0.8% | 99.8% | 4.4 | 0.007 | 0.6719 | 0.0588 | 0.0017 |
| Low red cell folate | 64 | | | | | | | 0.6416 | 0.0699 | 0.0214 |
| Low red cell Folate | 64 | 53.1% | 78.1% | 1.1% | 99.7% | 4.0 | 0.012 | 0.6563 | 0.0582 | 0.0036 |
| ROC | | | | | | | | | | |
| vitB12/vit D ROC | 64 | 87.5% | 46.9% | 0.7% | 99.9% | 6.2 | 0.005 | 0.6719 | 0.0538 | 0.0007 |
| HPL/Creatinine | 65 | | | | | | | 0.6903 | 0.0654 | 0.0018 |
| High HPL/ | 65 | 75.8% | 56.3% | 0.8% | 99.8% | 4.0 | 0.010 | 0.6600 | 0.0585 | 0.0031 |
| CreatinineROC | | | | | | | | | | |
| HPL/SG | 65 | | | | | | | 0.6165 | 0.0706 | 0.0495 |
| High HPL/SG ROC | 65 | 57.6% | 68.8% | 0.8% | 99.7% | 3.0 | 0.035 | 0.6316 | 0.0603 | 0.0145 |
| Serum B12 | 65 | | | | | | | 0.5218 | 0.0736 | 0.3835 |
| Free % Cu/Zn | 64 | | | | | | | 0.5493 | 0.0748 | 0.2549 |
| Free % Cu/Zn ROC | 64 | 34.4% | 93.8% | 2.4% | 99.7% | 7.9 | 0.012 | 0.6406 | 0.0479 | 0.0017 |
| High Serum B12 | 65 | 84.8% | 31.3% | 0.6% | 99.8% | 2.5 | 0.130 | 0.5805 | 0.0523 | 0.0619 |
| ROC | | | | | | | | | | |
| AD/MHMA | 63 | | | | | | | 0.8596 | 0.0468 | 0.0000 |
| AD/MHMA ROC | 63 | 84.8% | 80.0% | 1.9% | 99.9% | 22.4 | 0.000 | 0.8242 | 0.0488 | 0.0000 |
| AD/NA | 65 | | | | | | | 0.7083 | 0.0652 | 0.0007 |
| AD/NA ROC | 65 | 63.6% | 75.0% | 1.1% | 99.8% | 5.2 | 0.002 | 0.6932 | 0.0576 | 0.0004 |
| Plasma | 64 | | | | | | | 0.5103 | 0.0742 | 0.4448 |
| homocysteine | | | | | | | | | | |
| High Plasma | 64 | 90.9% | 25.8% | 0.6% | 99.8% | 3.5 | 0.088 | 0.5836 | 0.0473 | 0.0386 |
| homocysteine ROC | | | | | | | | | | |
| [vit B12 X % freeCu]/ | 62 | | | | | | | 0.6139 | 0.0773 | 0.0703 |
| [Zinc X folate x | | | | | | | | | | |
| vitB6] | | | | | | | | | | |
| [vit B12 X % freeCu | 62 | | | | | | | 0.6160 | 0.0774 | 0.0670 |
| X Homo cysteine]/ | | | | | | | | | | |
| [Zinc X folate X vit | | | | | | | | | | |
| B6] | | | | | | | | | | |
| [vit B12 X % freeCu | 61 | | | | | | | 0.6462 | 0.0786 | 0.0314 |
| XHomocysteine]/ | | | | | | | | | | |
| [Zinc X folate X vit | | | | | | | | | | |
| B6 X vitamin D] | | | | | | | | | | |
| Peak 1 amplitude/ | 61 | | | | | | | 0.5301 | 0.0748 | 0.6863 |
| Peak 2 amplitude | | | | | | | | | | |
| (Peak 2 amplitude/ | 61 | | | | | | | 0.5301 | 0.0748 | 0.6863 |
| Peak 1 amplitude | | | | | | | | | | |
| Creatinine ROC | 65 | | | | | | | 0.5578 | 0.0719 | 0.2107 |
| Urine B2/creatinine | 61 | | | | | 1.08 | 0.796 | 0.5215 | 0.0754 | 0.388 |
| level | | | | | | | | | | |
| Urine B2/creatinine | 61 | 67.7% | 46.7% | 0.6% | 99.7% | 1.84 | 0.252 | 0.5720 | 0.0630 | 0.127 |
| ROC | | | | | | | | | | |
| SIR Index | 65 | | | | | | | 0.9664 | 0.0303 | 0.0000 |
| GAF | 58 | | | | | | | 0.0006 | 0.0006 | 1.0000 |
| Hostility | 65 | | | | | | | 0.8788 | 0.0379 | 0.0000 |
| Suicidality | 65 | | | | | | | 0.7424 | 0.0442 | 0.0000 |
| Family history | 61 | 48.3% | 75.0% | 0.9% | 99.7% | 2.8 | 0.062 | 0.6164 | 0.0612 | 0.0286 |
| positive | | | | | | | | | | |
| Developmental | 62 | 73.3% | 100.0% | 100.0% | 99.9% | . | . | 0.8667 | 0.0411 | 0.0000 |
| delay | | | | | | | | | | |
| Learning disorder | 65 | 60.6% | 96.9% | 8.1% | 99.8% | 47.7 | 0.000 | 0.7874 | 0.0459 | 0.0000 |
| history | | | | | | | | | | |
| Head injury | 60 | 24.1% | 90.3% | 1.1% | 99.6% | 3.0 | 0.145 | 0.5723 | 0.0486 | 0.0684 |
| Ear infection history | 57 | 32.0% | 87.5% | 1.1% | 99.6% | 3.3 | 0.082 | 0.5975 | 0.0561 | 0.0411 |
| Bone conduction | 60 | 57.1% | 56.3% | 0.6% | 99.7% | 1.7 | 0.302 | 0.5670 | 0.0652 | 0.1521 |
| abnormality | | | | | | | | | | |
| Otoscopy | 59 | 14.8% | 75.0% | 0.3% | 99.5% | 0.5 | 0.338 | 0.4491 | 0.0522 | 0.8352 |

On ROC analysis, the under-methylating psychosis phenotype in a subject may also be associated with: (i) a positive family history of mental illness, history of developmental disorder, and/or history of learning disorder and/or history of ear infections; (ii) high overall symptom intensity index (SIR); and/or (iii) case-ness, duration of illness (DOI) and Global Assessment of Function (GAF) as an index of functional disability.

TABLE 14

| MTHFR wild | | | | | Outcome | |
|---|---|---|---|---|---|---|
| type CC | N | AUC | SE | P | Variable | Spearman's correlates |
| Vit B12/vit D | 64 | 0.7031 | 0.0644 | 0.0008 | Threshold | n 64, rho 0.352, P 0.004 |
| | | | | | for Caseness | |
| | | | | | DOI | n 60, rho 0.336, P 0.004 |
| | | | | | GAF | n 57, rho −0.336, P 0.011 |
| | | | | | Admission | n 60, rho 0.332, P 0.010 |
| | | | | | frequency* | |

The wild type MTHFR CC genotype is linked to 6 out of 6 sensory processing deficits—these being low visual span ROC and/or high ASOP age diff % ROC and/or high CW diff ROC (dichotic listening disorder) and/or low reverse digit span and/or low distance vision on the right ROC. On correlation analysis the wild type CC genotype is also linked to a large number of psychosis symptoms (in this study 38/42 symptoms), that include hostility and suicidality and comprise judgement and insight impairment, delusions, unusual thought content, suspiciousness, cognitive disorganization, emotional withdrawal, blunted affect, thought preoccupation, poor rapport, passivity/apathy, poor attention, hostility, excitement, abstract thinking impairment, lack of spontaneous conversation, social avoidance, anxiety, un-cooperativeness, hallucinations, bizarre behavior, disrupted volition, distractibility, self-neglect, depressed mood, poor impulse control, tension, motor retardation, suicidality, grandiosity, guilt, ideas reference and control, motor hyperactivity, somatic concern, elated mood, history of abuse, disorientation, mannerism and posturing, stereotypic thinking and/or experiencing blank periods.

On Spearman's correlation analysis, the under-methylating psychosis phenotype is associated with high likelihood of case-ness, symptom intensity (SIR), low global assessment of function (GAF) and or high Global Clinical Impression of illness (CGI), low social and occupational functioning (SOFAS), longer duration of illness (DOI), high admission frequency (admission number/DOI). On Spearman's correlation analysis), the under-methylating psychosis phenotype is also associated with high likelihood of low global assessment of function (GAF) and or high Global Clinical Impression of illness (CGI).

TABLE 15

| | | Spearman's | | |
|---|---|---|---|---|
| | | correlation | | |
| | Out- | coefficient (rho) | vitB12/ | Vit B12/ |
| | come | P (sig 2 tailed) | vitD | _folate |
| MTHFR | case | rho | .352 | .157 |
| negative | | P | .004 | .214 |
| 677 CC | | N | 64 | 64 |
| | SIR Index | rho | .338 | .182 |
| | | P | .006 | .151 |
| | | N | 64 | 64 |
| | GAF | rho | −.336 | −.312 |
| | | P | .011 | .018 |
| | | N | 57 | 57 |
| | CGI | rho | .386 | .258 |
| | | P | .003 | .053 |
| | | N | 57 | 57 |
| | SOFAS | rho | −.288 | −.247 |
| | | P | .030 | .064 |
| | | N | 57 | 57 |
| | Duration of | rho | .366 | .220 |
| | illness yrs | P | .004 | .095 |
| | | N | 60 | 59 |
| | Admit no | rho | .332 | .183 |
| | divided by | p | .010 | .166 |
| | DOI | N | 60 | 59 |

On logistic regression of ROC variables, for the MTHFR 677 CC variant, psychosis case-ness in the under-methylating psychosis phenotype may be predicted by: (i) elevated % free copper to zinc ratio relative to control values; (ii) elevated AD/MHMA levels/values relative to control values; (iii) elevated vitamin B12 levels relative to control levels/values; and/or (iv) low vitamin B6 levels/values relative to control level/values.

TABLE 16

| | | | | Number | | |
|---|---|---|---|---|---|---|
| Logistic regression | | | | of obs = 59 | | |
| MTHFR 677 | | | | LR | | |
| CC | | | | chi2(4) = 43.9 | | |
| | | | | Prob > | | |
| | | | | chi2 = 0 | | |
| Log likelihood = - | | | | Pseudo | | |
| 18.934775 | | | | R2 = 0.5369 | | |
| | Odds | | | | [95% | |
| case | Ratio | Std. Err. | z | P>\|z\| | Conf. | Interval] |
| free % cu/zn | 9.975525 | 11.82251 | 1.94 | 0.052 | 0.9775362 | 101.7979 |
| ROC | | | | | | |
| AD/MHMA | 25.40205 | 24.09701 | 3.41 | 0.001 | 3.957291 | 163.057 |
| ROC | | | | | | |
| vit B12/ | 21.29819 | 25.73367 | 2.53 | 0.011 | 1.994681 | 227.4113 |
| vit DROC | | | | | | |
| low B6 ROC | 12.17347 | 12.51401 | 2.43 | 0.015 | 1.623309 | 91.2909 |
| _cons | 0.0020782 | 0.0037968 | −3.38 | 0.001 | 0.0000579 | 0.0746153 |

Best ROC variable predictors of caseness in the MTHFR 677 CC variant are % free Copper to Zinc, AD/MHMA, vit B12/vit D, and low vit B6.

On logistic regression analysis, psychosis case-ness the under-methylating psychosis phenotype, incorporating continuous biomedical variables only, is typically and/or best predicted by elevated serum vitamin B12, high 5-HIAA, elevated AD+NA/MHMA and low vitamin B6. Prediction may be represented by number of abnormal variables and/or type of variables.

TABLE 17

| Logistic regression | | | | Number of obs = 58 | | |
|---|---|---|---|---|---|---|
| MTHFR 677 CC | | | | LR chi2(4) = 46.18 | | |
| | | | | Prob > chi2 = 0.0000 | | |
| Log likelihood = −17.11048 | | | | Pseudo R2 = 0.5744 | | |
| case | Coef. | Std. Err. | z | P > \|z\| | [95% Conf. Interval] | |
| seb12 | .0081122 | .004129 | 1.96 | 0.049 | .0000195 | .0162049 |
| hiaa | .8395397 | .5462907 | 1.54 | 0.124 | −.2311704 | 1.91025 |
| adna_mhma | .1895006 | .0628928 | 3.01 | 0.003 | .0662329 | .3127682 |
| b6 | −.0324021 | .0154171 | −2.10 | 0.036 | −.0626191 | −.002185 |
| _cons | −4.845206 | 2.231548 | −2.17 | 0.030 | −9.21896 | −.4714526 |

Note: 0 failures and 3 successes completely determined.
seb12 = serum vitamin B12, hiaa = 5-HIAA = 5 hydroxy inole acetic
acid, adna_mhma = AD + NA/MHMA, b6 = vitamin B6.
predict p5
(option pr assumed; Pr(case))
(9 missing values generated)
recode p5 (0.5/max = 1)(min/0.5 = 0), gen(predcase5)
(120 differences between p5 and predcase5)
diagt case predcase5

| | RECODE of p5 | | |
|---|---|---|---|
| | (Pr(case)) | | |
| Neg. | case | Pos. | Total |
| Abnormal | 49 | 15 | 64 |
| Normal | 10 | 51 | 61 |
| Total | 59 | 66 | 125 |

True abnormal diagnosis defined as case = 1
[95% Confidence Interval]

| Prevalence | Pr(A) | 51% | 42% | 60.2% |
|---|---|---|---|---|
| Sensitivity | Pr(+\|A) | 76.6% | 64.3% | 86.2% |
| Specificity | Pr(−\|N) | 83.6% | 71.9% | 91.8% |
| ROC area | (Sens. + Spec.)/2 | .801 | .731 | .871 |
| Likelihood ratio (+) | Pr(+\|A)/Pr(+\|N) | 4.67 | 2.61 | 8.36 |
| Likelihood ratio (−) | Pr(−\|A)/Pr(−\|N) | .28 | .178 | .443 |
| Odds ratio | LR(+)/LR(−) | 16.7 | 6.89 | 40.3 |
| Positive predictive value | Pr(A\|+) | 83.1% | 71% | 91.6% |
| Negative predictive value | Pr(N\|−) | 77.3% | 65.3% | 86.7% |

On logistic regression analysis, case-ness for psychosis in the under-methylating MTHFR 677 CC phenotype, incorporating biomedical variables and sensory processing variables together, is typically and/or best predicted with 84.3% sensitivity and 88.3% specificity by low vitamin B6, elevated visual speed of processing (age addition), elevated [AD+NA]/MHMA, and elevated auditory speed of processing (age difference). Prediction may be represented by number of abnormal variables and/or type of variables and/or by algorithm.

TABLE 18

| Logistic regression | | | | Number of obs = 53 | | |
|---|---|---|---|---|---|---|
| MTHER 677 CC | | | | LR chi2(4) = 55.66 | | |
| | | | | Prob > chi2 = 0.0000 | | |
| Log likelihood = −8.6718922 | | | | Pseudo R2 = 0.7624 | | |
| case | Coef. | Std. Err. | z | P > \|z\| | [95% Conf. Interval] | |
| b6 | −.1142906 | .0574237 | −1.99 | 0.047 | −.226839 | −.0017423 |
| vsopageadd | .2011612 | .0901302 | 2.23 | 0.026 | .0245093 | .3778132 |
| adna_mhma | .3507729 | .2046379 | 1.71 | 0.087 | −.0503101 | .7518559 |
| asopagediff | .24459 | .1200311 | 2.04 | 0.042 | .0093334 | .4798466 |
| _cons | 7.651535 | 4.735082 | 1.62 | 0.106 | −1.629054 | 16.93212 |

Note: 5 failures and 9 successes completely determined.
b6 = vitamin B6, vsopageadd = visual speed of processing (age
addition), adna_mhma = [AD + NA]/MHMA, asop age diff = auditory
speed of processing (age difference)
predict p6
(option pr assumed; Pr(case))
(23 missing values generated)
recode p6 (0.5/max = 1)(min/0.5 = 0), gen(predcase6)
(94 differences between p6 and predcase6)
diagt case predcase6

| | RECODE of p6 | | |
|---|---|---|---|
| | (Pr(case)) | | |
| case | Pos. | Neg. | Total |
| Abnormal | 43 | 8 | 51 |
| Normal | 7 | 53 | 60 |
| Total | 50 | 61 | 111 |

True abnormal diagnosis defined as case = 1

[95% Confidence Interval]

| Prevalence | Pr(A) | 46% | 36% | 55.7% |
|---|---|---|---|---|
| Sensitivity | Pr(+\|A) | 84.3% | 71.4% | 93% |
| Specificity | Pr(−\|N) | 88.3% | 77.4% | 95.2% |
| ROC area | (Sens. + Spec.)/2 | .863 | .798 | .928 |
| Likelihood ratio (+) | Pr(+\|A)/Pr(+\|N) | 7.23 | 3.57 | 14.6 |
| Likelihood ratio (−) | Pr(−\|A)/Pr(−\|N) | .178 | .0934 | .338 |
| Odds ratio | LR(+)/LR(−) | 40.7 | 13.8 | 120 |
| Positive predictive value | Pr(A\|+) | 86% | 73.3% | 94.2% |
| Negative predictive value | Pr(N\|−) | 86.9% | 75.8% | 94.2% |

On logistic regression analysis, case-ness for psychosis in the under-methylating MTHFR 677 CC phenotype, incorporating biomedical variables, sensory processing variables and urine vitamin B2-related variables, is typically and/or best predicted with 91.3% sensitivity and 96.6% specificity by low vitamin B6, elevated visual speed of processing (age addition) and elevated urine Peak 1 amplitude/Peak 2 amplitude related to vitamin B2 urinalysis as representative of vitamin B2 levels. Prediction may be represented by number of abnormal variables and/or type of variables and/or by algorithm.

TABLE 19

| | |
|---|---|
| Logistic regression | Number of obs = 52 |
| MTHFR 677 CC | LR chi2(4) = 53.14 |
| | Prob > chi2 = 0.0000 |

| | |
|---|---|
| Log likelihood = −9.1254004 | Pseudo R2 = 0.7444 |

| case | Odds Ratio | Std. Err. | z | P > \|z\| | [95% Conf. Interval] | |
|---|---|---|---|---|---|---|
| b6 | .8823792 | .0585312 | −1.89 | 0.059 | .7748045 | 1.00489 |
| hiaa | 4.648869 | 6.156117 | 1.16 | 0.246 | .3468731 | 62.30515 |
| Peak1/2ratio | .0069923 | .0239855 | −1.45 | 0.148 | 8.41e−06 | 5.813968 |
| vsop | 1.268583 | .1368149 | 2.21 | 0.027 | 1.026876 | 1.567184 |
| _cons | 12990.68 | 67159.95 | 1.83 | 0.067 | .5164647 | 3.27e+08 |

Note: 5 failures and 4 successes completely determined.
diagt case predcase10 if mthfrpoly==2
b6 = vitamin B6, hiaa = 5 HIAA,
peak ratio = Peak 1 amplitude/peak 2 amplitude,
vsop = visual speed of processing (age addition)

| | |
|---|---|
| (Pr(case)) | RECODE of p10 |

| case | Pos. | Neg. | Total |
|---|---|---|---|
| Abnormal | 21 | 2 | 23 |
| Normal | 1 | 28 | 29 |
| Total | 22 | 30 | 52 |

True abnormal diagnosis defined as case = 1

| | | | [95% Confidence Interval] | |
|---|---|---|---|---|
| Prevalence | Pr(A) | 44% | 30% | 58.7% |
| Sensitivity | Pr(+\|A) | 91.3% | 72% | 98.9% |
| Specificity | Pr(−\|N) | 96.6% | 82.2% | 99.9% |
| ROC area | (Sens. + Spec.)/2 | .939 | .871 | 1 |
| Likelihood ratio (+) | Pr(+\|A)/Pr(+\|N) | 26.5 | 3.84 | 182 |
| Likelihood ratio (−) | Pr(−\|A)/Pr(−\|N) | .0901 | .0239 | .339 |
| Odds ratio | LR(+)/LR(−) | 294 | 29.3 | . |
| Positive predictive value | Pr(A\|+) | 95.5% | 77.2% | 99.9% |
| Negative predictive value | Pr(N\|−) | 93.3% | 77.9% | 99.2% |

On logistic regression analysis, incorporating urine vitamin B2-related variables with other biomedical variables, the under-methylating MTHFR 677 CC psychosis phenotype may be predicted with 93.3% sensitivity and 92.6% specificity, by low vitamin B6, elevated [AD+NA}/MHMA, elevated 5HIAA and elevated Peak 1 amplitude/Peak 2 amplitude related to vitamin B2 urinalysis as representative of vitamin B2 levels. Prediction may be represented by number of abnormal variables and/or type of variables and/or by algorithm.

TABLE 20
Excluding sensory variables

| | |
|---|---|
| Logistic regression | Number of obs = 57 |
| MTHFR 677 CC | LR chi2(4) = 45.72 |
| | Prob > chi2 = 0.0000 |

| Log likelihood = −16.572295 | | | | Pseudo R2 = 0.5797 | | |
|---|---|---|---|---|---|---|
| case | Odds Ratio | Std. Err. | z | P > \|z\| | [95% Conf. Interval] | |
| b6 | .974799 | .0122398 | −2.03 | 0.042 | .9511021 | .9990863 |
| adna₋mhma | 1.25044 | .0909248 | 3.07 | 0.002 | 1.084347 | 1.441974 |
| hiaa | 1.816841 | .6930404 | 1.57 | 0.118 | .8602464 | 3.837167 |
| peakratio | .1738954 | .1643242 | −1.85 | 0.064 | .0272858 | 1.108255 |
| _cons | .2041357 | .3062405 | −1.06 | 0.290 | .0107885 | 3.862558 |

Note: 0 failures and 1 success completely determined.

| | |
|---|---|
| | B6 = vitamin B6, |
| | adna_mhma = AD + NA/MHMA, |
| | hiaa = 5-HIAA, |
| | peak ratio = Peak 2 amplitude/peak |
| diagt case predcase10 if mthfrpoly==2 | amplitude |

| | RECODE of p10 | | |
|---|---|---|---|
| | (Pr(case)) | | |
| case | Pos. | Neg. | Total |
| Abnormal | 28 | 2 | 30 |
| Normal | 2 | 25 | 27 |
| Total | 30 | 27 | 57 |

True abnormal diagnosis defined as case = 1

[95% Confidence Interval]

| Prevalence | Pr(A) | 53% | 39% | 66% |
|---|---|---|---|---|
| Sensitivity | Pr(+\|A) | 93.3 | 77.9% | 99.2% |
| Specificity | Pr(−\|N) | 92.6 | 75.7% | 99.1% |
| ROC area | (Sens. + Spec.)/2 | .93 | .862 | .997 |
| Likelihood ratio (+) | Pr(+\|A)/Pr(+\|N) | 12.6 | 3.31 | 48 |
| Likelihood ratio (−) | Pr(−\|A)/Pr(−\|N) | .072 | .0188 | .276 |
| Odds ratio | LR(+)/LR(−) | 175 | 24.8 | 1234 |
| Positive predictive value | Pr(A\|+) | 93.3% | 77.9% | 99.2% |
| Negative predictive value | Pr(N\|−) | 92.6% | 75.7% | 99.1% |

### Example 6—MTHFR 677 TT Genotype Indicative of an Over-Methylation Psychosis Phenotype

Based on statistical analysis of biomarker levels in accordance with the above-described examples, the inventors have identified that the presence of the MTHFR 677 TT homozygous variant is associated with an over-methylating phenotype. ROC analysis revealed that this over-methylating psychosis phenotype is associated with one or more of the following:
elevated AD/NA ratio, compared to control values;
elevated [vit B12 X % free Cu X Homocysteine]/[Zinc X folate X vitB6];
elevated [vitamin B12 X % free Cu X Homocysteine]/[Zinc X folate X vit B6 X vitamin D];
elevated levels of serum B12, compared to control levels/values;
elevated urine vitamin B2 (riboflavin)/creatinine, relative to control levels;
elevated % free copper to zinc ratio, compared to control values;
elevated oxidative stress as represented by both high HPL/SG and HPL/creatinine;
Abnormality of external ear drum detected on otoscopy;
lower histamine levels;
elevated Symptom Intensity rating (SIR), compared to control values;
low Global Assessment of Function (GAF) compared to control values;
elevated intensity rating for symptom of hostility, compared to control values;
elevated urine B2/creatinine level, compared to control values;
elevated B2 ug/L, compared to control values;
elevated Peak 2 amplitude/Peak 1 amplitude, compared to control values; and/or
elevated Peak 2 area/Peak 1 area, compared to control values, where (Peak 2=riboflavin and Peak1=riboflavin degradation product).

On correlation analysis, the over-methylating psychosis phenotype signature of elevated % free copper to zinc is linked to low zinc, (n 7, rho 1.000, P 0.000), since % free copper holds a strong inverse correlate with plasma zinc.

TABLE 21

| Variable for | | | | | | | | | | |
|---|---|---|---|---|---|---|---|---|---|---|
| homozygous MTHFR | | | | | | | OR p | | | |
| 677 TT | n | Sensitivity | Specificity | PPV | NPV | OR | value | AUC | se | P |
| Urine B2/creatinine | 6 | | | | | . | . | 1.0000 | 0.0000 | 0.000 |
| level | | | | | | | | | | |
| Urine B2/creatinine | 6 | 100.0% | 100.0% | 100.0% | 100.0% | . | . | 1.0000 | 0.0000 | 0.000 |
| ROC | | | | | | | | | | |
| B2 ug/L | 6 | | | | | | | 1.0000 | 0.0000 | 0.000 |
| Peak 2 amplitude/ | 6 | | | | | | | 1.0000 | 0.0000 | 0.000 |
| Peak 1 amplitude | | | | | | | | | | |
| Peak 2 area/Peak 1 | 6 | | | | | | | 1.0000 | 0.0000 | 0.000 |
| area | | | | | | | | | | |
| HPL/Creatinine | 7 | | | | | | | 0.9167 | 0.0833 | 0.0000 |
| Creatinine ROC | 7 | | | | | | | 0.7500 | 0.2500 | 0.1587 |
| high HPLCreatinine | 7 | 100.0% | 75.0% | 1.8% | 100.0% | . | . | 0.8750 | 0.1250 | 0.0013 |
| ROC | | | | | | | | | | |
| HPL/SG | 7 | | | | | | | 0.8333 | 0.1667 | 0.0228 |
| High HPL/SG ROC | 7 | 100.0% | 75.0% | 1.8% | 100.0% | . | . | 0.8750 | 0.1250 | 0.0013 |
| % free Cu/Zn | 7 | | | | | | | 0.9167 | 0.0833 | 0.0000 |
| plasma zinc | | | | | | | | | | |
| AD/NA | 7 | | | | | | | 0.1667 | 0.1863 | 0.0368 |
| DA X 5HIAA ROC | 7 | 100.0% | 50.0% | 0.9% | 100.0% | . | . | 0.7500 | 0.1143 | 0.0144 |
| High serum B12 ROC | 7 | 100.0% | 50.0% | 0.9% | 100.0% | . | . | 0.7500 | 0.1443 | 0.0416 |
| SIR Index | 7 | | | | | | | 1.0000 | 0.0000 | 0.0000. |
| [vit B12 X % free Cu]/ | 7 | | | | | | | 1.0000 | 0.0000 | 0.0000 |
| [Zinc X folate x vit B6] | | | | | | | | | | |
| [vit B12 X % free Cu X | 7 | | | | | | | 1 | 0.0000 | 0.0000 |
| Hocysteine]/[Zinc X | | | | | | | | | | |
| folate X vit B6] | | | | | | | | | | |
| Hmocysteine]/[Zinc X | 7 | | | | | | | 1 | 0.0000 | 0.0000 |
| folate X vit B6 X | | | | | | | | | | |
| vitamin D] | | | | | | | | | | |
| GAF | 7 | | | | | | | 0.0000 | 0.0000 | 0.000 |
| Hostility | 7 | | | | | | | 1.0000 | 0.0000 | 0.0000. |
| Otoscopy | 7 | 100.0% | 100.0% | 100.0% | 100.0% | . | . | 1.0000 | 0.0000 | 0.0 |
| 5HIAA | 7 | | | | | | | 0.5000 | 0.2635 | 0.5000 |
| 5HIAAROC | 7 | 33.3% | 100.0% | 100.0% | 99.7% | . | . | 0.6667 | 0.1667 | 0.1587 |
| DA x 5HIAA | 7 | | | | | 1.0071 | 0.481 | 0.6667 | 0.2357 | 0.2397 |
| NA/DA | 7 | | | | | | | 0.4167 | 0.2500 | 0.3695 |
| NA/DA ROC | 7 | 33.3% | 75.0% | 0.6% | 99.6% | 1.5 | 0.810 | 0.5417 | 0.2083 | 0.4207 |
| AD/NA ROC | 7 | 0.0% | 75.0% | 0.0% | 99.4% | . | . | 0.3750 | 0.1250 | 0.1587 |
| NA/MHMA | 7 | | | | | | | 0.5833 | 0.2500 | 0.3695 |
| NA/MHMA ROC | 7 | 33.3% | 100.0% | 100.0% | 99.7% | . | . | 0.6667 | 0.1667 | 0.1587 |
| Free Cu/Zn ROC | 7 | 66.7% | 75.0% | 1.2% | 99.8% | 6.0 | 0.287 | 0.7083 | 0.2083 | 0.1587 |
| AD/MHMA | 7 | | | | | | | 0.4167 | 0.2500 | 0.3695 |
| AD/MHMA ROC | 7 | 66.7% | 50.0% | 0.6% | 99.7% | 2.0 | 0.661 | 0.5833 | 0.2205 | 0.3528 |
| vit B12/vitD | 7 | | | | | | | 0.3333 | 0.2357 | 0.2397 |
| vit B12/vit DROC | 7 | 33.3% | 100.0% | 100.0% | 99.7% | . | . | 0.6667 | 0.1667 | 0.1587 |
| Plasma Homocysteine | 7 | | | | | | | 0.7083 | 0.2394 | 0.1921 |
| pllasma homocysteine | | | | | | | | | | |
| ROC | 7 | 66.7% | 75.0% | 1.2% | 99.8% | 6.0 | 0.287 | 0.7083 | 0.2083 | 0.1587 |
| Histamine | 7 | | | | | | | 0.5417 | 0.2917 | 0.4432 |
| Low histamine (<=0.35) | 7 | 33.3% | 100.0% | 100.0% | 99.7% | . | . | 0.6667 | 0.1667 | 0.1587 |
| High Histamine | | | | | | | | | | |
| (>=0.59) | 7 | 66.7% | 25.0% | 0.4% | 99.4% | 0.7 | 0.810 | 0.4583 | 0.2083 | 0.4207 |
| Histamine ROC | 7 | 66.7% | 75.0% | 1.2% | 99.8% | 6.0 | 0.287 | 0.7083 | 0.2083 | 0.1587 |
| Vit D | 7 | | | | | | | 0.3333 | 0.3333 | 0.3085 |
| Vit D ROC | 7 | 33.3% | 100.0% | 100.0% | 99.7% | . | . | 0.6667 | 0.1667 | 0.1587 |
| rcfolate | 7 | | | | | | | 0.5833 | 0.2500 | 0.3695 |
| Folate ROC | 7 | 66.7% | 75.0% | 1.2% | 99.8% | 6.0 | 0.287 | 0.7083 | 0.2083 | 0.1587 |
| Serum B12 | 7 | | | | | | | 0.5833 | 0.2500 | 0.3695 |
| vitamin B6 | 7 | | | | | | | 0.3333 | 0.2357 | 0.2397 |
| low vitamin B6 ROC | 7 | 33.3% | 100.0% | 100.0% | 99.7% | . | . | 0.6667 | 0.1667 | 0.1587 |
| Suicidality | 7 | | | | | | | 0.6667 | 0.1667 | 0.1587 |
| Family history positive | 7 | 0.0% | 75.0% | 0.0% | 99.4% | . | . | 0.3750 | 0.1250 | 0.1587 |
| Developmental delay | 7 | 66.7% | 75.0% | 1.2% | 99.8% | 6.0 | 0.287 | 0.7083 | 0.2083 | 0.1587 |
| Learning disorder | 7 | 66.7% | 75.0% | 1.2% | 99.8% | 6.0 | 0.287 | 0.7083 | 0.2083 | 0.1587 |
| history | | | | | | | | | | |
| Head injury | 7 | 33.3% | 100.0% | 100.0% | 99.7% | . | . | 0.6667 | 0.1667 | 0.1587 |
| Ear infection history | 7 | 33.3% | 100.0% | 100.0% | 99.7% | . | . | 0.6667 | 0.1667 | 0.1587 |
| Bone conduction | 7 | 66.7% | 75.0% | 1.2% | 99.8% | 6.0 | 0.287 | 0.7083 | 0.2083 | 0.1587 |
| abnormality | | | | | | | | | | |

On logistic regression of ROCs, the over-methylating psychosis phenotype demonstrates a relative absence of pathological predictors, being typically only predicted by abnormality of the external ear drum detected on clinical otoscopy and/o by elevated levels of urine riboflavin, compared with control values.

TABLE 22
tab case B2ROC if mthfrpoly == homozygous = MTHFR 677 TT

| | B2ROC | | |
|---|---|---|---|
| case | 0 | 1 | Total |
| 0 | 4 | 0 | 4 |
| 1 | 0 | 2 | 2 |
| Total | 4 | 2 | 6 |
| | B2ROC | | |
| case | Pos. | Neg. | Total |
| Abnormal | 2 | 0 | 2 |
| Normal | 0 | 4 | 4 |
| Total | 2 | 4 | 6 |

True abnormal diagnosis defined as case = 1

| | | [95% Confidence | | |
|---|---|---|---|---|
| Interval] | | | | |
| Prevalence | Pr(A) | .45% | ----- (given) ----- | |
| -- | | | | |
| Sensitivity | Pr(+\|A) | 100% | 15.8% | 100% |
| Specificity | Pr(−\|N) | 100% | 39.8% | 100% |
| ROC area | (Sens. + Spec.)/2 | 1 | 1 | 1 |
| Likelihood ratio (+) | Pr(+\|A)/Pr(+\|N) | . | . | . |
| Likelihood ratio (−) | Pr(−\|A)/Pr(−\|N) | 0 | . | . |
| Odds ratio | LR(+)/LR(−) | . | 2.29 | . |
| Positive predictive value | Pr(A\|+) | 100% | .% | .% (1r) |
| Negative predictive value | Pr(N\|−) | 100% | .% | .% (1r) |
| Pre-test odds | prev/(1-prev) | .0045 | ----- (given) ----- | |
| Post-test odds (+) | Pr(A\|+)/(1-Pr(A\|+)) | . | . | . (1r) |
| Post-test odds (−) | Pr(A\|−)/(1-Pr(A\|−)) | 0 | . | . (1r) |

| | |
|---|---|
| Logistic | MTHFR |
| Regression | 677 TT |

Otoscopy abnormality identifies cases
perfectly, no other variable has a statistically
significant relationship with case-ness.

| | otoscopy | | |
|---|---|---|---|
| case | No | Yes | Total |
| 0 | 4 | 0 | 4 |
| 1 | 0 | 3 | 3 |
| Total | 4 | 3 | 7 |

On correlation analysis, the over-methylating psychosis phenotype is associated with a lesser number of symptoms (27 out of 42) compared with the number of symptoms relating to other MTHFR 677 gene variants. The homozygous TT genotype is associated with only one sensory processing disorder out of six, this being impaired (abnormally high scoring) distance vision on the right. It is also associated with blunted affect, distractibility, hallucinations, hostility, anxiety, somatic concern, tension, delusions, judgement and insight impairment, excitement, grandiosity, poor impulse control, thought preoccupation, unusual thought, depressed mood, poor rapport, emotional withdrawal, social avoidance, passivity/apathy, self-neglect, lack of spontaneity conversation, uncooperativeness, bizarre behavior, elated mood, motor hyperactivity, and/or sense of outside self.

### Example 7—MTHFR 677 CT Genotype Indicative of a Mixed-Methylation Psychosis Phenotype

Based on statistical analysis of biomarker levels in accordance with the above-described examples, the inventors have identified that the presence of the MTHFR 677 CT heterozygous variant is associated with a mixed under- and over-methylating phenotype (i.e. is discriminated or characterized by markers representing a mixture of both the under-methylation and over-methylation phenotypes.

On ROC analysis, the mixed under- and over-methylating psychosis phenotype is typically discriminated and/or characterised by markers representing a mixture of both under-methylation and over-methylation phenotypes, including:
elevated Vitamin B12 divided by [red cell folate];
elevated [Vit B12 X Cu]_/[zn X folate]; and/or
elevated [Vit B12 X % free Cu X homocysteine]\[Zn X folate X vit B6].

TABLE 23

| MTHFR | | | | | | |
|---|---|---|---|---|---|---|
| heterozygous | | | | | Outcome | |
| 677 CT | N | AUC | SE | P | variable | Spearnman's correlates |
| Vitamin B12 | 62 | 0.6618 | 0.0693 | 0.0098 | Caseness | n 62, rho 0.280, P 0.027 |
| divided by [red | | | | | SIR | n 61, rho 0.280, P 0.027 |
| cell folate] | | | | | DOI | n 61, rho 0.321, P 0.012 |
| | | | | | GAF | n 59, rho −0.261, P 0.046 |
| [Vit B12 X cu]_/ | 62 | 0.6618 | 0.0695 | 0.0100 | Threshold of | N 62, rho 0.302, P 0.018 |
| [zn X folate] | | | | | hospital | (= hospital admission |
| | | | | | admission | number /DOI) |
| | | | | | frequency | |
| [vit B12 X % free | 58 | 0.6694 | 0.0724 | 0.0096 | Caseness | n 62, rho 0.277, P 0.029 |
| Cu X | | | | | DOI | n 61, rho 0.282, P 0.028 |
| homocysteine]/ | | | | | Admission | n 58, rho 0.302, P 0.019 |
| [Zinc X folate X | | | | | frequency* | |
| vit B6] | | | | | | |

On ROC analysis, the mixed psychosis phenotype is also typically discriminated and/or characterised by other markers representing both under-methylating and over-methylating phenotypes, risk factors and symptoms of suicidality and hostility.

TABLE 24

| Variable for MTHFR 677 | | | | | | | OR p | | | |
|---|---|---|---|---|---|---|---|---|---|---|
| CT | n | Sensitivity | Specificity | PPV | NPV | OR | value | AUC | se | P |
| 5H1AA | 61 | | | | | | | 0.6086 | 0.0683 | 0.0559 |
| High 5HIAAROC | 61 | 30.0% | 93.5% | 2.1% | 99.7% | 6.2 | 0.028 | 0.6177 | 0.0481 | 0.0072 |
| NA/DA | 61 | | | | | | | 0.7876 | 0.0580 | 0.0000 |
| High NA/DA ROC | 61 | 80.0% | 67.7% | 1.1% | 99.9% | 8.4 | 0.000 | 0.7387 | 0.0566 | 0.0000 |
| NA/MHMA | 60 | | | | | | | 0.8144 | 0.0567 | 0.0000 |
| High NA/MHMA ROC | 60 | 76.7% | 80.0% | 1.7% | 99.9% | 13.1 | 0.000 | 0.7833 | 0.0541 | 0.0000 |
| High Histamine ROC | 62 | 45.2% | 77.4% | 0.9% | 99.7% | 2.8 | 0.064 | 0.6129 | 0.0593 | 0.0285 |
| Vit D | 61 | | | | | | | 0.6269 | 0.0723 | 0.0396 |
| Low Vit D ROC | 61 | 70.0% | 58.1% | 0.7% | 99.8% | 3.2 | 0.030 | 0.6403 | 0.0620 | 0.0118 |
| red cell folate | 62 | | | | | | | 0.6774 | 0.0686 | 0.0049 |
| Low red cell Folate ROC | 62 | 67.7% | 64.5% | 0.9% | 99.8% | 3.8 | 0.013 | 0.6613 | 0.0611 | 0.0041 |
| Serum B12 | 62 | | | | | | | 0.6098 | 0.0727 | 0.0655 |
| High B12 ROC | 62 | 54.8% | 67.7% | 0.8% | 99.7% | 2.6 | 0.076 | 0.6129 | 0.0623 | 0.0350 |
| low B6 ROC | 59 | 76.7% | 44.8% | 0.6% | 99.8% | 2.7 | 0.085 | 0.6075 | 0.0612 | 0.0395 |
| HPL/Creatinine | 61 | | | | | | | 0.6935 | 0.0709 | 0.0032 |
| High HPLCreatinine | 61 | 66.7% | 74.2% | 1.2% | 99.8% | 5.7 | 0.002 | 0.7043 | 0.0593 | 0.0003 |
| ROC | | | | | | | | | | |
| HPL/SG | 61 | | | | | | | 0.6651 | 0.0726 | 0.0115 |
| High HPL/SG ROC | 61 | 73.3% | 71.0% | 1.1% | 99.8% | 6.7 | 0.001 | 0.7215 | 0.0583 | 0.0001 |
| DA x 5HIAA | 61 | | | | | 1.0041 | 0.0100 | 0.7059 | 0.0666 | 0.0010 |
| DA X 5HIAA ROC | 61 | 46.7% | 93.5% | 3.2% | 99.7% | 12.687 | 0.0002 | 0.7011 | 0.0515 | 0.0000 |
| vit B12/vit D | 61 | | | | | | | 0.6118 | 0.0724 | 0.0613 |
| High vit B12/vit DROC | 61 | 76.7% | 48.4% | 0.7% | 99.8% | 3.1 | 0.045 | 0.6253 | 0.0602 | 0.0187 |
| AD/NA | 61 | | | | | | | 0.6032 | 0.0741 | 0.0819 |
| High AD/NA ROC | 61 | 43.3% | 87.1% | 1.5% | 99.7% | 5.2 | 0.012 | 0.6522 | 0.0553 | 0.0030 |
| AD/MHMA | 60 | | | | | | | 0.7439 | 0.0650 | 0.0001 |
| High AD/MHMA ROC | 60 | 50.0% | 96.7% | 6.4% | 99.8% | 29.0 | 0.002 | 0.7333 | 0.0493 | 0.0000 |
| % Free Cu/Zn | 62 | | | | | | | 0.6337 | 0.0713 | 0.0304 |
| High % Free Cu/Zn | 62 | 77.4% | 41.9% | 0.6% | 99.8% | 2.5 | 0.107 | 0.5968 | 0.0590 | 0.0504 |
| ROC | | | | | | | | | | |
| Plasma Homocysteine | 61 | | | | | | | 0.5952 | 0.0736 | 0.0979 |
| High plasma | 61 | 63.3% | 58.1% | 0.7% | 99.7% | 2.4 | 0.097 | 0.6070 | 0.0635 | 0.0460 |
| homocysteine ROC | | | | | | | | | | |
| Histamine | 62 | | | | | | | 0.5937 | 0.0724 | 0.0978 |
| Low histamine (<=0.35) | 62 | 6.5% | 90.3% | 0.3% | 99.5% | 0.6 | 0.643 | 0.4839 | 0.0351 | 0.3232 |
| High Histamine (>=0.59) | 62 | 67.7% | 35.5% | 0.5% | 99.6% | 1.2 | 0.788 | 0.5161 | 0.0611 | 0.3961 |
| Low creatinine ROC | | | | | | | | | | |
| Urine B2/creatinine level | 55 | | | | | 0.90 | 0.433 | 0.5333 | 0.0814 | 0.341 |
| Urine B2/creatinine ROC | 55 | 56.0% | 66.7% | 0.8% | 99.7% | 2.55 | 0.095 | 0.6133 | 0.0670 | 0.045 |
| Vitamin B6 | 59 | | | | | | | 0.5833 | 0.0768 | 0.1390 |
| SIR Index | 62 | | | | | | | 0.9677 | 0.0323 | 0.0000 |
| GAF | 59 | | | | | | | 0.0006 | 0.0006 | 0.0000 |
| Hostility | 62 | | | | | | | 0.8710 | 0.0399 | 0.0000 |
| Suicidality | 62 | | | | | | | 0.6129 | 0.0382 | 0.0016 |
| Family history positive | 62 | 32.3% | 71.0% | 0.5% | 99.6% | 1.2 | 0.783 | 0.5161 | 0.0595 | 0.3934 |
| Developmental disorder | 62 | 46.7% | 87.1% | 1.6% | 99.7% | 5.9 | 0.006 | 0.6688 | 0.0555 | 0.0012 |
| Learning disorder history | 62 | 48.4% | 87.1% | 1.7% | 99.7% | 6.3 | 0.004 | 0.6774 | 0.0549 | 0.0006 |
| Head injury | 62 | 48.4% | 80.6% | 1.1% | 99.7% | 3.9 | 0.019 | 0.6452 | 0.0582 | 0.0063 |
| Ear infection history | 62 | 32.3% | 67.7% | 0.5% | 99.6% | 1.0 | 1.000 | 0.5000 | 0.0603 | 0.5000 |
| Bone conduction | 61 | 60.0% | 64.5% | 0.8% | 99.7% | 2.7 | 0.058 | 0.6226 | 0.0631 | 0.0260 |
| abnormality | | | | | | | | | | |
| Otoscopy abnormality | 61 | 23.3% | 74.2% | 0.4% | 99.5% | 0.9 | 0.823 | 0.4876 | 0.0560 | 0.4124 |

On Spearman's correlation analysis, the mixed methylating psychosis phenotype may contain an overlapping under-methylating phenotype signature together with the over-methylating phenotype signature.

TABLE 25
MTHFR 677 (CT) heteroxygous

| | | | | | | | | | [vit |
|---|---|---|---|---|---|---|---|---|---|
| | | | | | | | [vit | [vit | B12 × |
| | Spearman's | | | | | [vit | B12 × | B12 × | cu × hcy]/ |
| | correlation | | | | | B12 × | cu]/ | cu × hcy]/ | [zn × |
| | coefficient | | | | vit B12/ | cu]/ | [zn × | [zn × | folate × |
| | (rho)/P (sig | vit B12/ | vit B12/ | vit B12/ | [zn × | [zn × | folate × | folate × | vit B6 × |
| Outcome | 2 tailed | vit D | folate | zinc | folate] | folate] | vit B6] | vit B6] | vit D] |
| case | rho | .194 | .280 | .179 | .280 | .277 | .259 | .294 | .277 |
| | P | .135 | .027 | .163 | .027 | .029 | .048 | .025 | .037 |
| | N | 61 | 62 | 62 | 62 | 62 | 59 | 58 | 57 |
| SIR index | rho | .242 | .353 | .300 | .362 | .288 | .244 | .300 | .305 |
| | P | .060 | .005 | .018 | .004 | .023 | .063 | .022 | .021 |
| | N | 61 | 62 | 62 | 62 | 62 | 59 | 58 | 57 |
| GAFf | rho | −.138 | −.261 | −.142 | −.263 | −.207 | −.153 | −.194 | −.186 |
| | P | .302 | .046 | .283 | .044 | .115 | .260 | .155 | .179 |
| | N | 58 | 59 | 59 | 59 | 59 | 56 | 55 | 54 |
| CGI | rho | .185 | .277 | .177 | .270 | .220 | .132 | .181 | .193 |
| | P | .165 | .033 | .179 | .038 | .095 | .333 | .185 | .162 |
| | N | 58 | 59 | 59 | 59 | 59 | 56 | 55 | 54 |
| SOFAS | rho | −.102 | −.237 | −.098 | −.241 | −.240 | −.206 | −.245 | −.241 |
| | P | .445 | .070 | .459 | .066 | .067 | .128 | .071 | .079 |
| | N | 58 | 59 | 59 | 59 | 59 | 56 | 55 | 54 |
| Duration of | rho | .163 | .321 | .244 | .335 | .282 | .245 | .273 | .230 |
| Illness (yrs) | P | .214 | .012 | .058 | .008 | .028 | .064 | .040 | .087 |
| | N | 60 | 61 | 61 | 61 | 61 | 58 | 57 | 56 |

On ROC analysis, the mixed psychosis phenotype may be characterized by markers mainly representing the under-methylating component of this phenotype:
elevated 5HIAA and/or High 5HIAAROC, and/or
elevated NA/DA and or High NA/DA ROC, and/or
elevated NA/MHMA and/or High NA/MHMA ROC, and/or
elevated % Free Cu/Zn and/or High % Free Cu/Zn ROC, and/or
elevated/MHMA and/or high AD/MHMA ROC, and/or
elevated vitB12/vit D ROC, and/or
elevated Histamine ROC, and/or
low vitamin D and/or low Vit D ROC
low red cell folate and/or low red cell Folate ROC
elevated vitamin B12 ROC, and/or
low vitamin B6 ROC, and/or
HPL/Creatinine and/or High HPLCreatinineROC
HPL/SG and/or High HPL/SG ROC
elevated creatinine ROC, and/or
elevated AD/NA ROC (an over-methylating component, due to SAMe metabolizing NA to AD.)

On ROC analysis, the mixed psychosis phenotype may be characterized by risk factors of this phenotype, including history of developmental disorder, history of learning disorder history, history of subclinical head injury, and/or finding of subclinical bone conduction abnormality on auditory examination.

On ROC analysis, the mixed psychosis phenotype may be characterized by outcome of this phenotypes, including: SIR Index; GAF; elevated intensity of symptom of hostility; and/or elevated intensity of symptom of suicidality.

On logistic regression analysis, the mixed psychosis phenotype may be predicted by markers representing mainly the under-methylating component of this phenotype, including:
high NA/MHMA;
High NA/MHMA ROC;
low folate ROC;
high HPL/SG ROC; and/or
history of subclinical head injury.

On logistic regression analysis, the mixed psychosis phenotype incorporating both biomedical and risk factor variables, is associated with and predicted by high NA/MHMA, high AD/MHMA, low folate or high HPL/SG.

TABLE 26
Logistic regression

| | |
|---|---|
| MTHFR 677 heterozygous | Number of obs = 6000.0% |
| (CT) | LR chi2(5) = 4863.0% |
| | Prob > chi2 = 0 |
| | Pseudo R2 = 0.5847 |
| | Log likelihood = −17.272504 |

| | Odds | | | | | |
|---|---|---|---|---|---|---|
| case | Ratio | Std. Err. | z | P > \|z\| | [95% Conf. Interval] | |
| high NA/MHMA ROC | 5.638589 | 5.267537 | 1.85 | 0.064 | 0.903614 | 35.18504 |
| high AD/MHMA ROC | 17.86101 | 26.71416 | 1.93 | 0.054 | 0.9523355 | 334.9823 |
| low folate ROC | 28.8012 | 35.6443 | 2.72 | 0.007 | 2.546627 | 325.7285 |
| high HPL/SG ROC | 18.30844 | 20.31033 | 2.62 | 0.009 | 2.081502 | 161.037 |
| subclinical head injury | 12.75411 | 14.18934 | 2.29 | 0.022 | 1.440994 | 112.8855 |
| _cons | 0.0033709 | 0.0057227 | −3.35 | 0.001 | 0.000121 | 0.0939301 |

On logistic regression analysis, psychosis case-ness for carriers of the mixed MTHFR 677 CT psychosis phenotype incorporating biomedical, risk factor and sensory processing variables, may be predicted with 90% sensitivity and 95% specificity by: elevated [histamine+NA], elevated vitamin D/vitamin B12, positive history of learning disorder, increased visual speed of processing, and/or reduced competing words difference.

TABLE 27
Logistic regression

| | |
|---|---|
| MTHFR 677 CT | Number of obs = 55 |
| | LR chi2 (6) = 55.95 |
| | Prob > chi2 = 0.0000 |
| | Pseudo R2 = 0.7382 |
| | Log likelihood = −9.9214602 |

| case | Coef. | Std. Err. | z | P > \|z\| | [95% Conf. Interval] | |
|---|---|---|---|---|---|---|
| HistamineNA | .0753733 | .0386492 | 1.95 | 0.051 | −.0003779 | .1511244 |
| vitD_vitB12ROC | 2.266755 | 1.558607 | 1.45 | 0.146 | −.7880592 | 5.321569 |
| ld_hist | 1.615572 | 1.41458 | 1.14 | 0.253 | −1.156953 | 4.388097 |
| asoptrim | .1036894 | .0638997 | 1.62 | 0.105 | −.0215518 | .2289305 |
| vsoptrim | .1263032 | .0680721 | 1.86 | 0.064 | −.0071156 | .259722 |
| cwdiff | −.0404953 | .0405208 | −1.00 | 0.318 | −.1199147 | .0389241 |
| _cons | −3.655753 | 1.718859 | −2.13 | 0.033 | −7.024655 | −.2868516 |

HistamineNA = [Histamine + NA], vitD_vit B12 ROC =
Vitamin D/Vitamin B12 ROC, ld hist = history of
learning disorder, asopagediff = auditory speed of
processing (age difference), vsopageadd = visual speed of
processing (age addition), cw diff = competing words
difference = dichotic listening disorder.
predict p4
(option pr assumed; Pr(case))
(18 missing values generated)
recode p4 (0.5/max = 1) (min/0.5 = 0), gen(predcase4)
(116 differences between p4 and predcase4)
diagt case predcase4

| | RECODE of p4 | | |
|---|---|---|---|
| | (Pr(case)) | | |
| case | Pos. | Neg. | Total |
| Abnormal | 45 | 5 | 50 |
| Normal | 3 | 63 | 66 |
| Total | 48 | 68 | 116 |

True abnormal diagnosis defined as case = 1

| | | [95% Confidence Interval] | | |
|---|---|---|---|---|
| Prevalence | Pr(A) | 43% | 34% | 52.6% |
| Sensitivity | Pr(+\|A) | 90% | 78.2% | |
| 96.7% | | | | |
| Specificity | Pr(−\|N) | 95.5% | 87.3% | |
| 99.1% | | | | |
| ROC area | (Sens. + Spec.)/2 | .927 | .878 | .976 |
| Likelihood ratio (+) | Pr(+\|A)/Pr(+\|N) | 19.8 | 6.53 | 60 |
| Likelihood ratio (−) | Pr(−\|A)/Pr(−\|N) | .105 | .0455 | .241 |
| Odds ratio | LR(+)/LR(−) | 189 | 44.6 | 791 |
| Positive predictive value | Pr(A\|+) | 93.8 | 82.8% | 98.7% |
| Negative predictive value | Pr(N\|−) | 92.6% | 83.7% | 97.6% |

On Logistic regression analysis, psychosis case-ness for carriers of the mixed MTHFR 677 CT psychosis phenotype incorporating biomedical variables, risk factor variables, sensory processing variables and vitamin B2 related urine analysis, may be predicted with 88.5% sensitivity and 93.3% specificity by elevated [histamine+NA], positive history of learning disorder, increased auditory speed of processing, increased visual speed of processing, and/or increased competing words difference (representing dichotic listening disorder).

TABLE 28
Logistic regression

| | |
|---|---|
| MTHFR 677 CT | Number of obs = 56 |
| | LR chi2 (6) = 54.58 |
| | Prob > chi2 = 0.0000 |
| | Pseudo R2 = 0.7056 |
| | Log likelihood = −9.9214602 |

| case | Odds Ratio | Std. Err. | z | P > \|z\| | [95% Conf. Interval] | |
|---|---|---|---|---|---|---|
| HistamineNA | 1.097878 | .0432921 | 2.37 | 0.018 | 1.016224 | 1.186094 |
| ld_hist | 11.27436 | 14.43341 | 1.89 | 0.058 | .9170349 | 138.6111 |
| asopagediff | 1.020658 | 0.267622 | 0.78 | 0.435 | .9695298 | 1.074482 |
| vsopageadd | 1.115 | .0562443 | 2.16 | 0.031 | 1.010037 | 1.23087 |
| cwdiff | .9741027 | .0288531 | −0.89 | 0.376 | .919162 | 1.032327 |
| _cons | .0451402 | .0555287 | −2.52 | 0.012 | .0040501 | .5031068 |

HistamineNA = [Histamine + NA], vitD_vit B12 ROC = Vitamin D/Vitamin B12 ROC,
ld hist = history of learning disorder,
asopagediff = auditory speed of processing (age difference),
vsopageadd = visual speed of processing (age addition),
cw diff = competing words difference = dichotic listening disorder.
diagt case predcase10 if mthfrpoly == 1

| | RECODE of p10 | | |
|---|---|---|---|
| | (Pr(case)) | | |
| case | Pos. | Neg. | Total |
| Abnormal | 23 | 3 | 26 |
| Normal | 2 | 28 | 30 |
| Total | 25 | 31 | 56 |

True abnormal diagnosis defined as case = 1

| | | [95% Confidence Interval] | | |
|---|---|---|---|---|
| Prevalence | Pr(A) | 46% | 33% | 60.3% |
| Sensitivity | Pr(+\|A) | 88.5% | 69.8% | |
| 97.6% | | | | |
| Specificity | Pr(−\|N) | 93.3% | 77.9% | |
| 99.2% | | | | |
| ROC area | (Sens. + Spec.)/2 | .909 | .832 | .986 |
| Likelihood ratio (+) | Pr(+\|A)/Pr(+\|N) | 13.3 | 3.45 | 51 |
| Likelihood ratio (−) | Pr(−\|A)/Pr(−\|N) | .124 | .0425 | .36 |
| Odds ratio | LR(+)/LR(−) | 107 | 17.7 | 640 |
| Positive predictive value | Pr(A\|+) | 92% | 74% | 99% |
| Negative predictive value | Pr(N\|−) | 90.3% | 74.2% | 98% |

The heterozygous CT genotype is associated with 41 out of 42 symptoms and 5 out of 6 sensory processing deficits that include low reverse digit span ROC, high CW diff ROC, high ASOP age diff % ROC, high Distance vision on right ROC and low visual span ROC. Symptoms include impaired judgement and Insight impairment, suspiciousness, cognitive disorganization, delusions, unusual thought, blunted affect, poor attention, abstract thinking impairment, distractibility, thought preoccupation, hallucinations, hostility, poor impulse control, emotional withdrawal, anxiety, excitement, passivity/apathy, uncooperativeness, grandiosity, self-neglect, lack spontaneous conversation, bizarre behavior, depressed mood, poor rapport, motor hyperactivity, disturbed volition, elated mood, social avoidance, disorientation, ideas reference and control, motor retardation, stereotypic thinking, somatic concern, tension, suicidality, experiencing blank periods, unreal feelings, history of abuse, guilt, mannerisms and posturing, feeling as if outside of self. The heterozygous CT genotype is also associated with high levels of function outcome measures and/or indices for severity, disability and cost and/or care burden and high numbers of symptoms representative of psychosis.

On Spearman's correlation analysis, a mixed or overlapping methylating state may co-occur within any MTHFR 677 C->T variant phenotype. This mixed status may be represented by single markers and/or mixed over-methylation and/or under—methylation markers occurring together in compound marker(s). Such a mixed signature is characterised by elevated B12/zinc X folate, and/or elevated [vitamin B12 X % free copper]_/[zinc X folate], and/or [vitamin B12 X % free copper]_/[zinc X folate X B6] and/or [vitamin B12 X % free Cu X homocysteine]/[zinc X folate], and/or [vitamin B12 X % free Cu X homocysteine]/[zinc X folate X vitamin B6 X vitamin D], where low folate, B6 or low vitamin D markers are typical of biochemistry operating in an under-methylating status, whereas low zinc, high homocysteine high serum vitamin B12 is more typical of biochemistry operating in an over-methylating status.

TABLE 29
MTHFR 677 (CT) heteroxygous

| | | | | | | | [vit |
|---|---|---|---|---|---|---|---|
| | | | | | [vit | [vit | B12 × |
| | Spearman's | | | [vit | B12 × | B12 × | cu × hcy]/ |
| | correlation | | | B12 × | cu]/ | cu × hcy]/ | [zn × |
| | coefficient | | vit B12/ | cu]/ | [zn × | [zn × | folate × |
| | (rho)/P (sig | vit B12/ | [zn × | [zn × | folate × | folate × | vit B6 × |
| Outcome | 2 tailed | zinc | folate] | folate] | vit B6] | vit B6] | vit D] |
| case | rho | .069 | .195 | .110 | .197 | .201 | .253 |
| | P | .586 | .123 | .387 | .124 | .117 | .049 |
| | N | 65 | 64 | 64 | 62 | 62 | 61 |
| SIR index | rho | .120 | .194 | −.046 | −.009 | −.005 | .010 |
| | P | .340 | .125 | .721 | .946 | .967 | .938 |
| | N | 65 | 64 | 64 | 62 | 62 | 61 |
| GAF | rho | −.120 | −.332 | −.010 | −.129 | −.148 | −.196 |
| | P | .368 | .012 | .944 | .346 | .280 | .155 |
| | N | 58 | 57 | 57 | 55 | 55 | 54 |
| CGI | rho | .108 | .276 | .097 | .213 | .223 | .254 |
| | P | .418 | .038 | .473 | .119 | .102 | .063 |
| | N | 58 | 57 | 57 | 55 | 55 | 54 |
| SOFAS | rho | −.138 | −.258 | .042 | −.095 | −.115 | −.133 |
| | P | .302 | .053 | .757 | .488 | .402 | .336 |
| | N | 58 | 57 | 57 | 55 | 55 | 54 |
| Duration of | rho | .148 | .255 | .097 | .194 | .200 | .212 |
| Illness (yrs) | P | .259 | .051 | .464 | .147 | .137 | .113 |
| | N | 60 | 59 | 59 | 57 | 57 | 57 |
| Admit no. | rho | .092 | .202 | .177 | .257 | .274 | .287 |
| divided | P | .483 | .126 | .181 | .054 | .039 | .030 |
| By DOI | N | 60 | 59 | 59 | 57 | 57 | 57 |

On Spearman's correlation analysis, in the mixed methylating psychosis phenotype the elevated vitamin B12/[zinc X folate] marker is significantly correlated and/or linked with markers of disability and illness severity and longevity such as high SIR and/or low GAF and/or low SOFAS and/or high CGI and/or long DOI, whereas elevated [vitamin B12 X % free copper]_/[zinc X folate X B6] and/or [vitamin B12 X % free Cu X homocysteine]/[zinc X folate], and/or [vitamin B12 X % free Cu X homocysteine]/[zinc X folate X vitamin B6 X vitamin D] markers are not associated or linked significantly to severity and disability outcome measures, but are rather linked significantly with outcome measures representing illness instability with acute outcome as represented by high hospital admission frequency as represented by an individual's hospital admission number divided by their duration of their illness in years.

On correlation analysis, the mixed methylating psychosis phenotype and/or signature, of elevated [vitamin B12 X % free Cu] divided by [plasma Zinc X red cell folate X vitamin B6] becomes marginally characterized by, linked to or associated with low global assessment of function (GAF) (n 7, rho−0.741, P 0.057) in the presence of elevated % free copper to zinc (n 7, rho 0.722, P 0.067), and/or elevated AD/NA or DA).

### Example 8—Duration of Illness

In order to advantage longitudinal monitoring, longitudinal management, longer term prevention of psychosis progression and/or progression towards partial or complete remission and/or recovery from schizophrenia and/or schizoaffective psychosis, the inventors have determined biomedical markers, sensory processing markers and risk factor markers that predict duration of illness (DOI) for schizophrenia and/or schizoaffective disorder. DOI is associated, linked and predicted by the following markers, determined for each MTHFR 677 C->T variant, using Mann Whitney U test, logistic regression analysis and high strength of Spearman's correlation for DOI (see Table below). As detailed in the above examples, labile and/or overlapping and/or mixed methylation phenotypes (concurrent under- and over-methylation signatures) is seen to persist across DOI to a small extent, for all the MTHFR 677 C->T gene variants.

TABLE 30

| MTHFR | | | | |
|---|---|---|---|---|
| 677 | Mann Whitney | | | Distinguishing |
| MTHFR | Predictors | | Logistic | Spearman's |
| 677Gene | z = prediction coefficient, | | Regression | Correlation |
| variant | Pz = significance of prediction | | analysis | Signature |
| CC | Family history | | % free Cu/Zn | Low vitamin D ROC |
| | History of ear infection | | ROC | High vit B12/vit D ROC |
| | Learning disorder | | AD/MHMA | High NA/MHMA. |
| | Developmental disorder. | | ROC | High NA/MHMA ROC |
| | Low red cell folate ROC | | Vitamin | High HPL/creatinine |
| | Low vitamin B6 ROC | | B12/vitamin D | 5HIAA in urine |
| | Low vitamin D ROC | | ROC | |
| | High vitamin B12 ROC | | Low vitamin B6 | |
| | High HPL/creatinine ROC | | ROC | |
| | High DA, NA | | | |
| | High NA/MHMA | | | |
| | High AD/MHMA ROCs | | | |
| | 5-HIAA in the urine. | | | |
| | VSOP age add ROC | N 60, z −5.614, Pz | | |
| | 0.000 | | | |
| | Visual span ROC | N 60, z 2.251, Pz 0.0244 | | |
| | Distance vision R ROC | N 60, z −3.212, Pz 0.0013 | | |
| | Digit span Backwards ROC | N 60, z 2.063, Pz 0.0391 | | |
| | Competing words agediff ROC | N 60, z −4.806, Pz 0.0000 | | |
| | ASOP agediffROC | N 60, z −4.257, Pz | | |
| | 0.0000 | | | |
| | HPL/SG ROC | N 60, z −2.123, Pz 0.0338 | | |
| | AD/NA ROC | N 60, z −2.980, Pz 0.0029 | | |
| | NA/MHMA ROC | N 58, z −3.834, Pz 0.0001 | | |
| | AD/MHMA ROC | N 58, z −4.593, Pz 0.0000 | | |
| | NA/DA ROC | N 60, z −4.257, P 0.0000 | | |
| | Vitamin B12/vitamin D ROC | N 60, z −2.848, P 0.0044 | | |
| CT | High NA/MHMA ROC | | NA/MHMA | NA/MHMA ROC |
| | AD/MHMA ROCs. | | ROC | AD/MHMA ROC |
| | High histamine ROC | | AD/MHMA | DA/NA ROC |
| | Low folate ROC | | ROC | Low RC folate ROC |
| | Low vitamin D ROC | | Low folate ROC | Low vitamin B6 ROC |
| | High 5HIAA ROC | | HPL/SG ROC | vitamin D levels normal |
| | High HPL/creatinine ROC | | Subclinical | 5HIAA in urine |
| | Sub-clinical head injury | | head injury | |
| | Learning disorder | | | |
| | Developmental disorder. | | | |
| | VSOP ROC | N 61, z −4.569, P 0.0000 | | |
| | DV on R | N 61, z −3.108, P 0.0019 | | |
| | ASOP ROC | N 61, z −4.930, P 0.0000 | | |
| | NA/DA ROC | N 60, z −3.633, P 0.0003 | | |
| | AD/NA ROC | N 60, z −2.668, P 0.0076 | | |
| | NA/MHMA ROC | N 59, z −4.211, P 0.0000 | | |
| | AD/MHMA ROC | N 59, z −3.907, P 0.0001 | | |
| | Vitamin B12/vitamin D ROC | N 60, z −1.743, P 0.0813 | | |
| | HPL/SG ROC | N 60, z −3.376, P 0.0007 | | |
| | HPL/SG ROC | N 60, z −3.376, Pz 0.0007 | | |
| | Distance vision R ROC | N 61, z −3.108, Pz 0.0019 | | |
| | NA/MHMA ROC | N 59, z −4.211, Pz 0.000 | | |
| | AD/MHMA ROC | N 59, z −3.907, Pz 0.0001 | | |
| | Vitamin B12/vitamin D ROC | N 60, z −1.743, Pz 0.0813 | | |
| TT | Low zinc ROC | | Otoscopy | |
| | Abnormality on otoscopy | | abnormality | |
| | HPL/SG | N 7, z −3.786, Pz 0.0002 | (No | |
| | HPL/SG ROC | N 7. Z −3.786, Pz 0.0002 | NA/MHMA) | |
| | Distance vision R ROC | N7, z −1.755. Pz 0.0792 | (No AD/MHMA | |
| | ASOP ROC | N 7, z −0.2341, P 0.0193 | ROCs) | |

DOI relates significantly to Vitamin B12 divided by [red cell folate] and/or [vitamin B12 X % free Cu X homocysteine]/[Zinc X folate X vitamin B6]

TABLE 31

| MTHFR | | | | | Spearnman's |
|---|---|---|---|---|---|
| heterozygous | | | | | correlates |
| 677 CT | N | AUC | SE | P | For DOI at 95% CI |
| Vitamin B12 | 62 | 0.6618 | 0.0693 | 0.0098 | n 61, rho |
| divided by | | | | | 0.321, P 0.012 |
| [red cell | | | | | |
| folate] | | | | | |
| [vit B12 × | 58 | 0.6694 | 0.0724 | 0.0096 | n 61, rho |
| % free Cu × | | | | | 0.282, P 0.028 |
| homocysteine]/ | | | | | |
| [Zinc × folate × | | | | | |
| vit B6] | | | | | |

### Example 9—Prognostic Indicators for the MTHFR 677 CC Variant

The inventors used logistic regression analysis to determine predictors of various functional outcomes, hostility and suicidality as prognostic indicators for the MTHFR 677 CC variant based on the biomarker analysis described in the above examples.

Predictors of duration of illness are: elevated catecholamine markers, noradrenaline and adrenaline; elevated visual processing markers: reduced visual span and aged, delayed speed of visual processing; and comorbidity factors: type 2 diabetes and hypertension (see Table X).

Predictors of elevated Symptom Intensity Rating (SIR) are: reduced visual span and delayed visual speed of processing; elevated % free copper to zinc ratio; and elevated [AreaP1/P2]/creatinine ROC representing excess riboflavin metabolites relative to riboflavin in urine, indicative of riboflavin degradation exceeding riboflavin synthesis (see Table X).

Predictors of elevated Clinical Global Impression (CGI) of illness severity are: elevated Vitamin B12/zinc X folate; elevated [AreaP1/P2]/creatinine ROC representing excess riboflavin metabolites relative to riboflavin in urine, indicative of riboflavin degradation exceeding riboflavin synthesis; delayed auditory speed of processing; and impaired distance vision on R (see Table X).

Predictors of Reduced Global Assessment of Function (GAF) are: serum B12/red cell folate representative of undermethylation or over-methylation signature; reduced visual span and delayed visual speed of processing; and reduced [Area P 1/P2]/creatinine ROC representing excess riboflavin metabolites relative to riboflavin in urine, indicative of riboflavin degradation exceeding riboflavin synthesis (see Table X).

Predictors of reduced Social and Occupational Function Scale (SOFAS) value are: reduced vitamin B6; elevated serum B12/zinc X folate; reduced visual span and visual speed of processing; and reduced [Area P1/P2]/creatinine ROC representing excess riboflavin metabolites relative to riboflavin in urine, indicative of riboflavin degradation exceeding riboflavin synthesis (see Table X).

Predictors of high admission frequency (admission number/DOI) are: visual speed of processing delay (see Table X).

Predictors of high requirement for disability support pension are: low vitamin B6; low visual span; and delayed auditory speed of processing (see Table X).

Predictors of high cost and care burden are: elevated [serum vitamin B12 x % free Cu X vitamin B6]/Zn x folate x vitamin B6, representative of under methylation phenotype with switch to over methylation component; elevated [Area P1/P2]/creatinine ROC] representing excess riboflavin metabolites relative to riboflavin in urine, indicative of riboflavin degradation exceeding riboflavin synthesis; and delayed visual speed of processing (see Table X).

Hostility predictors are: elevated serum vitamin B12; and reduced visual span (correlates with elevated AD and HPL) (see Table X).

Suicidality predictors are: elevated NA (switch from undermethylation to overmethylation); and reduced visual span (correlates with elevated AD (vigilance) and HPL) (see Table X).

TABLE 32
Duration of Illness - MTHER 677 CC

| Source | SS | df | MS | |
|---|---|---|---|---|
| Model | 2286.84468 | 7 | 326.692097 | Number of obs = 38 |
| Residual | 582.129008 | 30 | 19.4043003 | Prob > F = 0.0000 |
| Total | 2868.97368 | 37 | 77.5398293 | R-squared = 0.7971 |
| | | | | Adj R-squared = 0.7498 |
| | | | | Root MSE = 4.405 |

| Durationof~s | Coef. | Std. Err. | t | P > \|t\| | [95% Conf. Interval] | |
|---|---|---|---|---|---|---|
| NA | .0981144 | .0778114 | 1.26 | 0.217 | −.0607976 | .2570264 |
| Visual span | −1.464674 | .7668907 | −1.91 | 0.066 | −3.030874 | .1015257 |
| Hpl/creat | .4154953 | .1523064 | 2.73 | 0.011 | .1044442 | .7265465 |
| diabetes | 19.32686 | 6.291406 | 3.07 | 0.004 | 6.478094 | 32.17562 |
| vsop | .1143579 | .0390132 | 2.93 | 0.006 | .0346822 | .1940335 |
| adren | .7561976 | .3250931 | 2.33 | 0.027 | .0922688 | 1.420126 |
| hypertension | −5.810267 | 2.568128 | −2.26 | 0.031 | −11.05508 | −.5654498 |
| _cons | 7.370879 | 5.139812 | 1.43 | 0.162 | −3.126018 | 17.86778 |

TABLE 33
SIR Index - MTHFR 677 CC
sw, pr(.05): regress SIRIndex vspan vsoptrim freecuzn areap1_p2_creatROC if mthfrpoly == 2
begin with full model
p < 0.0500 for all terms in model

| Source | SS | df | MS | |
|---|---|---|---|---|
| Model | 40694.477 | 4 | 10173.6192 | Number of obs = 52 |
| Residual | 25612.0422 | 47 | 544.937069 | F(4, 41) = 18.67 |
| Total | 66306.5192 | 51 | 1300.12783 | Prob > F = 0.0000 |
| | | | | R-squared = 0.6137 |
| | | | | Adj R-squared = 0.5809 |
| | | | | Root MSE = 23.344 |

| SIR Index | Coef. | Std. Err. | t | P > \|t\| | [95% Conf. Interval] | |
|---|---|---|---|---|---|---|
| Visual span | −8.456504 | 2.766081 | −3.06 | 0.004 | −14.02114 | −2.981866 |
| vsop | .7971293 | .1599061 | 4.98 | 0.000 | .4754398 | 1.118819 |
| % free cu/zn | −13.98088 | 6.233408 | −2.24 | 0.030 | −26.52087 | −1.440877 |
| [AreaP1/P2]/creatROC | 17.1338 | 8.4581 | 2.03 | 0.048 | .1182972 | 34.1493 |
| _cons | .5079 | 18.76223 | 5.41 | 0.000 | 63.76313 | 139.2526 |

TABLE 34
CGI - MTHFR 677 CC
sw, pr(.05): regress cgi adren b6 dvr6 asoptrim b12_znfolate areap1_p2_creatROC if mthfrpol
p < 0.0500 for all terms in model

| Source | SS | df | MS | |
|---|---|---|---|---|
| Model | 136.890134 | 6 | 22.8150223 | Number of obs = 46 |
| Residual | 47.566879 | 39 | 1.21965097 | Prob > F = 0.0000 |
| Total | 184.456522 | 45 | 4.09903382 | R-squared = 0.7421 |
| | | | | Adj R-squared = 0.7025 |
| | | | | Root MSE = 1.1044 |

| cgi | Coef. | Std. Err. | t | P > \|t\| | [95% Conf. Interval] | |
|---|---|---|---|---|---|---|
| adren | .1473976 | .0408329 | 3.61 | 0.001 | .0648053 | .2299899 |
| Vitamin B6 | −.0068313 | .001871 | −3.65 | 0.001 | −.0106157 | −.0030468 |
| Impaired distance | .0607189 | .023443 | 2.59 | 0.013 | .0133009 | .1081369 |
| vision on R | | | | | | |
| Asop | .0401069 | .0073567 | 5.45 | 0.000 | .0252265 | .0549874 |
| Vit B12/zn X folate | 55.28554 | 25.39928 | 2.18 | 0.036 | 3.910649 | 106.6604 |
| [AreaP1/P2]/reatROC | 1.348602 | .4371989 | 3.08 | 0.004 | .4642843 | 2.232921 |
| _cons | .7306487 | .6061417 | 1.21 | 0.235 | −.4953886 | 1.956686 |

TABLE 35
GAF - MTHFR 677 CC
sw, pr(.05): regress gaf vspan vsoptrim b12_folate areap1_p2_creatROC if mthfrpoly == 2
begin with full model
p < 0.0500 for all terms in model

| Source | SS | df | MS | |
|---|---|---|---|---|
| Model | 24686.2287 | 4 | 6171.55716 | Number of obs = 46 |
| Residual | 9794.7496 | 41 | 238.896332 | F(4, 41) = 25.83 |
| Total | 34480.9783 | 45 | 766.243961 | Prob > F = 0.0000 |
| | | | | R-squared = 0.7159 |
| | | | | Adj R-squared = 0.6882 |
| | | | | Root MSE = 15.456 |

| gaf | Coef. | Std. Err. | t | P > \|t\| | [95% Conf. Interval] | |
|---|---|---|---|---|---|---|
| visual span | 6.895572 | 1.906555 | 3.62 | 0.001 | 3.045206 | 10.74594 |
| vsop | −.6486533 | .1127514 | −5.75 | 0.000 | −.8763595 | −.4209472 |
| B12/folate | −53.68371 | 26.31763 | −2.04 | 0.048 | −106.8332 | −.5341695 |
| [AreaP1/P2]/creatROC | −17.51464 | 5.919045 | −2.96 | 0.005 | −29.4684 | −5.560886 |
| _cons | 64.48403 | 14.25368 | 4.52 | 0.000 | 35.69814 | 93.26992 |

TABLE 36
SOFAS - MTHFR 677 CC
sw, pr(.05): regress soc b6 vspan vsoptrim areap1_p2_creatROC b12_znfolate if mthfrpoly == 2
begin with full model
p < 0.0500 for all terms in model

| Source | SS | df | MS | |
|---|---|---|---|---|
| Model | 27972.3163 | 5 | 5594.46325 | Number of obs = 44 |
| Residual | 7543.59282 | 38 | 198.515601 | F(5, 38) = 28.18 |
| Total | 35515.9091 | 43 | 825.951374 | Prob > F = 0.0000 |
| | | | | R-squared = 0.7876 |
| | | | | Adj R-squared = 0.7597 |
| | | | | Root MSE = 14.09 |

| sofas | Coef. | Std. Err. | t | P > \|t\| | [95% Conf. Interval] | |
|---|---|---|---|---|---|---|
| Vitamin B6 | .0869307 | .024331 | 3.57 | 0.001 | .0376709 | .1361905 |
| Visual span | 8.609857 | 1.909178 | 4.51 | 0.000 | 4.744928 | 12.47479 |
| vsop | −.4529255 | .1082723 | −4.18 | 0.000 | −.6721113 | −.2337397 |
| [AreaP1/P2]/creatROC | −21.75411 | 5.500349 | −3.96 | 0.000 | −32.88899 | −10.61924 |
| B12/zn X folate | −904.2138 | 326.593 | −2.77 | 0.009 | −1565.367 | −243.0608 |
| _cons | 51.66668 | 13.94378 | 3.71 | 0.001 | 23.43898 | 79.89438 |

TABLE 37
Admissions/DOI = MTHFR 677 CC
sw, pr(.05): regress AdmitnodividedbyDOI vsoptrim if mthfrpoly == 2
begin with full model
p < 0.0500 for all terms in model

| Source | SS | df | MS | |
|---|---|---|---|---|
| Model | 2.96094804 | 1 | 2.96094804 | Number of obs = 54 |
| Residual | 5.73485211 | 52 | .110285617 | F(1, 52) = 26.85 |
| Total | 8.69580015 | 53 | .164071701 | Prob > F = 0.0000 |
| | | | | R-squared = 0.3405 |
| | | | | Adj R-squared = 0.3278 |
| | | | | Root MSE = .33209 |

| Admitnodiv~I | Coef. | Std. Err. | t | P > \|t\| | [95% Conf. Interval] | |
|---|---|---|---|---|---|---|
| vsop | .0107198 | .0020689 | 5.18 | 0.000 | .0065683 | .0148712 |
| _cons | .1565654 | .0453505 | 3.45 | 0.001 | .065563 | .2475677 |

TABLE 38
Disability support pension - MTHFR 677 CC
sw, pr(.05): lr: logistic DspPension b6 vspan asoptrim if mthfrpoly == 2
LR test begin with full model
p < 0.0500 for all terms in model

| | |
|---|---|
| Logistic regression | Number of obs = 53 |
| | LR chi2(3) = 39.26 |
| | Prob > chi2 = 0.0000 |
| | Pseudo R2 = 0.5780 |
| | Log likelihood = −14.333375 |

| DspPension | Odds Ratio | Std. Err. | z | P > \|z\| | [95% Conf. Interval] | |
|---|---|---|---|---|---|---|
| Vitamin B6 | .9811318 | .0163187 | −1.15 | 0.252 | .9496635 | 1.013643 |
| visual span | .2882067 | .1246246 | −2.88 | 0.004 | .1234902 | .672629 |
| Asop | 1.072632 | .0268616 | 2.80 | 0.005 | 1.021255 | 1.126593 |
| _cons | 2442.115 | 6286.902 | 3.03 | 0.002 | 15.72029 | 379377.6 |

TABLE 39
Total cost and care burden (in term of both community pension support and
hospitalisation frequency index) for MTHFR 677 CC
sw, pr(.05): regress tccb vsoptrim b12cuhcy_znfolateb6 areap1_p2 if mthfrpoly == 2
begin with full model
p < 0.0500 for all terms in model

| Source | SS | df | MS | |
|---|---|---|---|---|
| Model | 18.3091886 | 3 | 6.10306286 | Number of obs = 49 |
| Residual | 12.6839362 | 45 | .281865248 | F(3, 45) = 21.65 |
| Total | 30.9931247 | 48 | .645690099 | Prob > F = 0.0000 |
| | | | | R-squared = 0.5908 |
| | | | | Adj R-squared = 0.5635 |
| | | | | Root MSE = .53091 |

| tccb | Coef. | Std. Err. | t | P > \|t\| | [95% Conf. Interval] | |
|---|---|---|---|---|---|---|
| Vsop | .0227862 | .003438 | 6.54 | 0.000 | .0157695 | 0.298029 |
| [B12XCuXHcy]/ | 15.06983 | 6.854278 | 2.20 | 0.033 | 1.264608 | 28.87506 |
| Zn X folate X B6 | | | | | | |
| AreaP1/P2 | −.0051518 | .0023624 | −2.18 | 0.034 | −.0099099 | −.0003937 |
| _cons | .2928811 | .1006648 | 2.91 | 0.006 | .0901317 | .4956305 |

TABLE 40
Hostility MTHFR 677 CC
sw, pr(.05): lr: logistic hostile seb12 vspan if mthfrpoly == 2
LR test begin with full model
p < 0.0500 for all terms in model

| | |
|---|---|
| Logistic regression | Number of obs = 58 |
| | LR chi2(2) = 15.28 |
| | Prob > chi2 = 0.0005 |
| | Pseudo R2 = 0.3053 |
| | Log likelihood = −17.389683 |

| hostile | Odds Ratio | Std. Err. | z | P > \|z\| | [95% Conf. Interval] | |
|---|---|---|---|---|---|---|
| Serum vit B12 | 1.006505 | .0027669 | 2.36 | 0.018 | 1.001097 | 1.011943 |
| Visual span | .4483922 | .1667634 | −2.16 | 0.031 | .2163138 | .9294627 |
| _cons | .5508576 | 1.146593 | −0.29 | 0.775 | .0093173 | 32.5679 |

TABLE 41
Suicidality - MTHFR 677 CC
logistic suicidal ad_na vspan if mthfrpoly == 2

| | |
|---|---|
| Logistic regression | Number of obs = 58 |
| | LR chi2(2) = 17.22 |
| | Prob > chi2 = 0.0002 |
| | Pseudo R2 = 0.3056 |
| | Log likelihood = −19.56381 |

| suicidal | Odds Ratio | Std. Err. | z | P > \|z\| | [95% Conf. Interval] | |
|---|---|---|---|---|---|---|
| AD/NA | 286.0965 | 898.637 | 1.80 | 0.072 | .6065483 | 134945.9 |
| Visual span | .3919372 | .1388944 | −2.64 | 0.008 | .1956899 | .7849908 |
| _cons | 7.912165 | 14.25082 | 1.15 | 0.251 | .2318314 | 270.034 |

1. A method for diagnosing a psychosis phenotype in a subject with a psychotic disorder, the method comprising:
(a) obtaining one or more biological samples from said subject, and
(b) determining the status of the C677T polymorphism of the MTHFR gene from the one or more biological samples,
wherein
(i) the presence of the homozygous CC genotype at position 677 of the MTHFR gene is indicative of an under-methylating psychosis phenotype,
(ii) the presence of the homozygous TT genotype at position 677 of the MTHFR gene is indicative of a low activity MTHFR enzyme and an over-methylating psychosis phenotype, and
(iii) the presence of the heterozygous CT genotype at position 677 of the MTHFR gene is indicative of a mixed-methylation psychosis phenotype.

2-26. (canceled)

27. The method according to claim 1, wherein the psychotic disorder is schizophrenia, schizoaffective disorder or psychosis.

28. The method according to claim 1, wherein the method further comprises the determination of levels of one or more biomarkers, and optionally the ratios of selected biomarkers, in the one or more biological samples, to inform the diagnosis.

29. The method according to claim 28, wherein the one or more biomarkers are selected from: free copper, zinc, indolamines and catecholamines and their metabolites, vitamin and mineral or trace element cofactors, intermediate substances, or vitamin B2 excretion levels.

30. The method according to claim 28, wherein the one or more biomarkers are selected from: free copper, zinc, vitamin D, riboflavin and flavin-related compounds, vitamin B6, vitamin B12, folate and related compounds, S-adenosylmethionine (SAMe), S-adenosylhomocysteine (SAH), hydroxylpyrolline-2-one (HPL), histamine, adrenaline (AD), noradrenaline (NA), 5-hydroxyondolacetic acid (5HIAA) or methylhydroxy vanillyl-mandelic acid (MHMA).

31. The method according to claim 1, wherein the biological sample(s) comprise blood and/or urine samples.

32. The method according to claim 28, comprising the measurement of riboflavin and flavin-related compounds in a urine sample.

33. The method according to claim 32, wherein the flavin-related compounds are riboflavin metabolites and degradation products, optionally FAD and/or FMN.

34. The method according to claim 32, wherein the method comprises determining the ratio of riboflavin degradation to riboflavin synthesis or the difference between riboflavin degradation and synthesis, in the urine sample.

35. The method according to claim 1, further comprising the assessment or measurement of one or more additional parameters, selected from measurement or assessment of one or more symptom ratings for schizophrenia, schizoaffective disorder or psychosis, risk factor analysis, functional visual and auditory acuity, external ear canal patency, tympanic membrane status, motor capacity, extrapyramidal or thyroid status.

36. The method according to claim 35, wherein the symptom ratings are measured or assessed using one or more psychiatric symptom rating scales selected from: the Brief Psychiatric Rating Scale (BPRS); the Positive and Negative Syndrome Scale (PANSS), the Global Assessment of Function (GAF) Scale; the Clinical Global Impressions (CGI) score; or the Social and Occupational Functioning Scale (SOFAS).

37. The method according to claim 35, wherein the risk factors for analysis are selected from risk factors with regard history of ear infection, developmental disorder or delay, family history of mental illness, history of clinical or subclinical head injury, history of abuse, and history of learning disorder.

38. The method according to claim 28, wherein the determination of biomarker levels, or ratios thereof, and the assessment of measurement of additional parameters is subjected to one or more statistical analyses.

39. The method according to claim 38, wherein the statistical analyses comprise one or more of receiver operating characteristic (ROC) analysis, logistic regression analysis, Spearman's rank correlation analysis, or the Mann-Whitney U test.

40. The method according to claim 28, wherein the method further comprises measuring the levels of one or more biomarkers, and optionally the ratios of selected biomarkers, and/or the one or more of said additional parameters, in one or more control individuals, wherein said control individuals are known not to suffer from the psychotic disorder.

41. A method for diagnosing a psychotic disorder, optionally schizophrenia, schizoaffective disorder or psychosis, in a subject, the method comprising:
(a) obtaining one or more biological samples from said subject,
(b) determining levels of one or more biomarkers, and optionally the ratios of selected biomarkers, in the one or more biological samples, and/or measuring or assessing one or more additional parameters, and
(c) optionally comparing determined, measured or assessed levels, values or ratios from (b) with corresponding control levels, values or ratios from one or more individuals known not to suffer from the psychotic disorder,
wherein the status of the C677T polymorphism of the MTHFR gene in the subject is known, or is determined from the one or more biological samples, and wherein
(i) the presence of the homozygous CC genotype at position 677 of the MTHFR gene is indicative of an under-methylating psychosis phenotype,
(ii) the presence of the homozygous TT genotype at position 677 of the MTHFR gene is indicative of a low activity MTHFR enzyme and an over-methylating psychosis phenotype, and
(iii) the presence of the heterozygous CT genotype at position 677 of the MTHFR gene is indicative of a mixed-methylation psychosis phenotype.

42. A method for predicting or determining expected duration of illness or prognosis of a subject with a psychotic disorder, the method comprising:
(a) obtaining one or more biological samples from said subject,
(b) determining the status of the C677T polymorphism of the MTHFR gene and the psychosis phenotype,
(c) determining levels of one or more biomarkers, and optionally the ratios of selected biomarkers,
(d) measuring or assessing one or more additional parameters, and
(e) determining expected duration of illness or prognosis from an analysis of said determined, measured or assessed biomarkers and additional parameters.

43. The method according to claim 42, wherein the additional parameters comprise expected or predicted functional outcome measures for one or more of SIR, GAF, OFAS, expected frequency of hospital admissions, CGI status, or DSP status.

44. A method for treating or preventing a psychotic disorder in a subject, or for alleviating one or more symptoms of said disorder, the method comprising:
(a) determining the MTHFR 677 genotype and psychosis phenotype of a subject in accordance with claim 1,
(b) optionally determining levels of one or more biomarkers, and optionally the ratios of selected biomarkers,
(c) optionally measuring or assessing one or more additional parameters, and
(d) determining a suitable treatment regime for the subject, wherein
(i) when the MTHFR 677 genotype of the subject is the wild-type (CC) genotype and the psychosis phenotype of the subject is under-methylating, the method comprises administering to the subject an effective amount of riboflavin, a prodrug analogue or derivative thereof, and/or an agent capable of inhibiting riboflavin degradation, and
(ii) when the MTHFR 677 genotype of the subject is the homozygous TT genotype and the psychosis phenotype of the subject is over-methylating, the method comprises administering to the subject an effective amount of niacin or nicotinamide, a prodrug analogue or derivative thereof.
